(19) United States
(12) Patent Application Publication
(10) Pub. No.: US 2022/0280543 A1
(43) Pub. Date: Sep. 8, 2022

(54) DDX17 AND NLRC4 TARGETING FOR INFLAMMATORY DISEASES

(71) Applicant: University of Virginia Patent Foundation, Charlottesville, VA (US)

(72) Inventors: Jayakrishna Ambati, Charlottesville, VA (US); Shao-bin Wang, Charlottesville, VA (US); Kameshwari Ambati, Charlottesville, VA (US)

(73) Assignee: University of Virginia Patent Foundation, Charlottesville, VA (US)

(21) Appl. No.: 17/637,557

(22) PCT Filed: Aug. 24, 2020
(86) PCT No.: PCT/US2020/047640

§ 371 Date: Feb. 23, 2022

Related U.S. Application Data
(60) Provisional application No. 62/891,124, filed on Aug. 23, 2019.

(51) Int. Cl. A61K 31/7076; A61P 27/02; A61K 31/675; A61K 31/513; A61K 31/7072; A61K 31/708; A61K 31/7068; A61K 31/685; C07K 16/18; C07K 16/40; C07K 16/28

(52) U.S. Cl. CPC A61K 31/7076; A61P 27/02; A61K 31/675; A61K 31/513; A61K 31/7072; A61K 31/708; A61K 31/7068; A61K 31/685; C07K 16/18; C07K 16/40; C07K 16/2866

(57) ABSTRACT

Provided are methods for treating and/or preventing diseases, disorders, and/or conditions associated with NLR family CARD domain containing 4 (NLRC4) inflammasome biological activities. In some embodiments, the method include administering to a subject in need thereof a composition that includes a nucleoside reverse transcriptase inhibitor (NRTI). Also provided are methods for inhibiting NLRC4-induced caspase-1 activation in cells, methods for inhibiting NLRC4-induced IL-Iβ release from cells, methods for inhibiting Alu-induced retinal pigmented cell (RPE) degeneration in subjects, and compositions for use in the presently disclosed methods.

### CROSS REFERENCE TO RELATED APPLICATION

The presently disclosed subject matter claims the benefit of U.S. Provisional Patent Application Ser. No. 62/891,124, filed Aug. 23, 2019, the disclosure of which incorporated herein by reference in its entirety.

### GOVERNMENT INTEREST

This invention was made with government support under grant numbers DP1GM114862 and R01EY029799 awarded by the National Institutes of Health. The government has certain rights in the invention.

### TECHNICAL FIELD

The presently disclosed subject matter relates generally to compositions and methods for treating and/or preventing a disease, disorder, or condition associated with an NLR family CARD domain containing 4 (NLRC4) inflammasome biological activity. In some embodiments, the methods comprise administering to a subject in need thereof a composition comprising, consisting essentially of, or consisting of a nucleoside reverse transcriptase inhibitor (NRTI), wherein the administering is via an route and in an amount effective for reducing the NLRC4 inflammasome biological activity.

### BACKGROUND

Nucleotide oligomerization domain (NOD) like receptors (NLRs) play a crucial role in the innate immune response to diverse stimuli. Some NLRs contribute to antibacterial immunity, e.g. the NLR family, CARD-containing 4 (NLRC4) inflammasome is activated by intracytoplasmic bacterial flagellin and T3SS components, thereby cleaving pro-caspase-1 into its active form to trigger pyroptosis and downstream inflammatory cascade. NLRC4 does not directly recognize these bacterial products; instead it utilizes the NLR family apoptosis inhibitor proteins (NAIP) family of proteins to sense flagellin and T3SS. NAIPs serve as direct receptors for bacterial ligands, thereby enabling the NLRC4 inflammasome as an adaptor for downstream inflammatory cascades. In mice, NAIPs 1-6 are capable of mediating NLRC4 activation in response to specific bacterial ligands. However, humans lack the duplication of the NAIP gene seen in mice; instead a single human NAIP enables the recognition of multiple bacterial ligands. Although sterile tissue damage is also known to activate the NLRC4 inflammasome in models of ischemic stroke and multiple sclerosis, it is unclear which endogenous stimuli activate the NLRC4 inflammasome in these settings in the absence of bacterial infection. The sensory spectrum of NLRC4 inflammasome for these diverse sterile activators is unknown and to date, there are no known NLRC4 inflammasome inhibitors.

Short interspersed nuclear elements (SINEs) are non-coding retrotransposons that comprise approximately 10% of the mammalian genome. Alu RNA is the most successful retrotransposon SINE element in humans, whereas B1, B2, ID, and B4 are mouse SINEs. Genomic insertion and/or transcriptional excess of SINEs can cause inflammasome activation, and are associated with multiple diseases including cystic fibrosis, hemophilia A, retinitis pigmentosa, age related macular degeneration (AMD), diabetes, and hypercholesterolemia. However, the upstream sensor for recognizing SINE RNAs is still unknown.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating and/or preventing a disease, disorder, and/or condition associated with an NLR family CARD domain containing 4 (NLRC4) inflammasome biological activity, the method comprising, consisting essentially of, or consisting of administering to a subject in need thereof a composition comprising, consisting essentially of, or consisting of a nucleoside reverse transcriptase inhibitor (NRTI), wherein the administering is via an route and in an amount effective for reducing the NLRC4 inflammasome biological activity, thereby treating and/or preventing the disease, disorder, or condition associated with the NLRC4 inflammasome biological activity. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the disease, disorder, or condition associated with the NLRC4 inflammasome biological activity is a disease of the retinal pigmented epithelium (RPE), optionally age-related macular degeneration (AMD) and/or geographic atrophy. In some embodiments, the disease, disorder, or condition associated with the NLRC4 inflammasome biological activity is selected from the group consisting of graft-versus-host disease, chronic pain, proliferative vitreoretinopathy, glaucoma, rheumatoid arthritis, multiple sclerosis, bipolar disorder, major depressive disorder, renal fibrosis, nephritis, pulmonary fibrosis, Huntington's disease, osteoporosis, chronic lymphocytic leukemia, anxiety disorders, pulmonary tuberculosis, osteoporosis in post-menopausal women and fracture patients, systemic lupus erythematosus, chronic inflammatory and neuropathic pain, autosomal dominant polycystic kidney disease, spinal cord injury, Alzheimer's disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson's disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., Leishmania spp, SARS-CoV, SARS-CoV-2 and Chlamydia spp., cryopyrinopathies; keratitis, acne vulgaris, Crohn's disease, ulcerative colitis, irritable bowel syndrome, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, chronic obstructive pulmonary disease, drug-induced lung inflammation, contact dermatitis, leprosy, Burkholderia cenocepacia infection, respiratory syncytial virus infection, psoriasis, scleroderma, reactive arthritis, cystic fibrosis, syphilis, Sjogren's syndrome, inflammatory joint disease, non-alcoholic fatty liver disease, cardiac surgery (peri-/post-operative inflammation), acute and chronic organ transplant rejection, acute and chronic bone marrow transplant rejection, and tumor angiogenesis.

In some embodiments, the presently disclosed methods further comprise, consist essentially of, or consist of administering to the subject in need thereof at least one additional inhibitor of the NLRC4 inflammasome biological activity. In some embodiments, the at least one additional inhibitor the NLRC4 inflammasome biological activity comprises, consists essentially of, or consists of an inhibitor of a biological activity of at least one molecule or complex selected from the group consisting of NLRC4, NLRP3, caspase-1 (CAS-1), cyclic GMP-AMP synthase (CGAS), caspase-4 (CAS-4), stimulator of interferon genes-1 (STING1), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR). In some embodiments, the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product. In some embodiments, the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

In some embodiments, the presently disclosed subject matter also relates to methods for inhibiting NLRC4-induced caspase-1 activation in a cell. In some embodiments, the methods comprise, consist essentially of, or consist of contacting an NLRC4 gene product and/or a complex of an NLRC4 gene product and an NLR family pyrin domain containing 3 (NLRP3) gene product with an effective amount of a composition comprising, consisting essentially of, or consisting of a nucleoside reverse transcriptase inhibitor (NRTI), whereby NLRC4-induced caspase-1 activation is inhibited in the cell. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the cell is present in a subject, optionally a mammalian subject, further optionally a human subject.

In some embodiments, the presently disclosed methods further comprise, consist essentially of, or consist of administering to the subject in need thereof at least one additional inhibitor of the NLRC4 inflammasome biological activity. In some embodiments, the at least one additional inhibitor the NLRC4 inflammasome biological activity comprises, consists essentially of, or consists of an inhibitor of a biological activity of at least one molecule or complex selected from the group consisting of NLRC4, NLRP3, caspase-1 (CAS-1), cyclic GMP-AMP synthase (CGAS), caspase-4 (CAS-4), stimulator of interferon genes-1 (STING1), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR). In some embodiments, the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product. In some embodiments, the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

The presently disclosed subject matter also relates in some embodiments to methods for inhibiting NLRC4-induced IL-1β release from a cell. In some embodiments, the methods comprise, consist essentially of, or consist of contacting an NLRC4 gene product and/or a complex of an NLRC4 gene product and an NLR family pyrin domain containing 3 (NLRP3) gene product with an effective amount of a composition comprising, consisting essentially of, or consisting of a nucleoside reverse transcriptase inhibitor (NRTI), whereby NLRC4-induced IL-1β release from the cell is inhibited. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the cell is present in a subject, optionally a mammalian subject, further optionally a human subject. In some embodiments, the NLRC4-induced caspase-1 activation and/or the NLRC4-induced IL-1β release is associated with a disease, disorder, and/or condition associated with an NLRC4 inflammasome biological activity. In some embodiments, the disease, disorder, or condition associated with the NLRC4 inflammasome biological activity is a disease of the retinal pigmented epithelium (RPE), optionally age-related macular degeneration (AMD) and/or geographic atrophy. In some embodiments, the disease, disorder, and/or condition associated with an NLRC4 inflammasome biological activity is selected from the group consisting of graft-versus-host disease, chronic pain, proliferative vitreoretinopathy, glaucoma, rheumatoid arthritis, multiple sclerosis, bipolar disorder, major depressive disorder, renal fibrosis, nephritis, pulmonary fibrosis, Huntington's disease, osteoporosis, chronic lymphocytic leukemia, anxiety disorders, pulmonary tuberculosis, osteoporosis in post-menopausal women and fracture patients, systemic lupus erythematosus, chronic inflammatory and neuropathic pain, autosomal dominant polycystic kidney disease, spinal cord injury, Alzheimer's disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson's disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., Leishmania spp, SARS-CoV, SARS-CoV-2 and Chlamydia spp., cryopyrinopathies; keratitis, acne vulgaris, Crohn's disease, ulcerative colitis, irritable bowel syndrome, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, chronic obstructive pulmonary disease, drug-induced lung inflammation, contact dermatitis, leprosy, Burkholderia cenocepacia infection, respiratory syncytial virus infection, psoriasis, scleroderma, reactive arthritis, cystic fibrosis, syphilis, Sjogren's syndrome, inflammatory joint disease, non-alcoholic fatty liver disease, cardiac surgery (peri-/post-operative inflammation), acute and chronic organ transplant rejection, acute and chronic bone marrow transplant rejection, and tumor angiogenesis

In some embodiments, the presently disclosed methods further comprise, consist essentially of, or consist of administering to the subject in need thereof at least one additional inhibitor of the NLRC4 inflammasome biological activity. In some embodiments, the at least one additional inhibitor is selected from the group consisting of an antisense oligonucleotide, a small interfering RNA (siRNA), a short hairpin RNA (shRNA), an antibody or antigen-binding fragment thereof. In some embodiments, the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

In some embodiments, the presently disclosed subject matter relates to methods for inhibiting Alu-induced retinal pigmented cell (RPE) degeneration in a subject. In some embodiments, the methods comprise, consist essentially of, or consist of contacting an NLRC4 gene product and/or a complex of an NLRC4 gene product and an NLR family pyrin domain containing 3 (NLRP3) gene product in a cell of the subject with an effective amount of a composition comprising, consisting essentially of, or consisting of a nucleoside reverse transcriptase inhibitor (NRTI), whereby NLRC4-induced IL-1β release from the cell is inhibited. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the cell is an RPE cell that present in a subject, optionally a mammalian subject, further optionally a human subject.

In some embodiments, the presently disclosed methods further comprise, consist essentially of, or consist of administering to the subject at least one additional treatment designed to protect the RPE from degradation. In some embodiments, the at least one additional treatment comprises administering to the subject an inhibitor of a biological activity of at least one molecule or complex selected from the group consisting of NLRC4, NLRP3, caspase-1, cyclic GMP-AMP synthase (cGAS), caspase-4, stimulator of interferon genes (STING), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR). In some embodiments, the inhibitor is a small interfering RNA (siRNA) or short hairpin RNA (shRNA) that targets a transcription product of a gene selected from the group consisting of NLRC4, NLRP3, caspase-1, cGAS, caspase-4, STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product. In some embodiments, the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

The presently disclosed subject matter also relates in some embodiments to compositions for use in treating and/or preventing diseases, disorders, and/or conditions associated with NLRC4 inflammasome biological activities. In some embodiments, the compositions comprise, consist essentially of, or consist of a nucleoside reverse transcriptase inhibitor (NRTI).

The presently disclosed subject matter also relates in some embodiments to compositions for use in inhibiting NLRC4-induced IL-1β release from a cell, the composition comprising, consisting essentially of, or consisting of a nucleoside reverse transcriptase inhibitor (NRTI).

The presently disclosed subject matter also relates in some embodiments to compositions for use in inhibiting Alu-induced retinal pigmented cell (RPE) degeneration in a subject, the composition comprising, consisting essentially of, or consisting of a nucleoside reverse transcriptase inhibitor (NRTI).

In some embodiments of the presently disclosed compositions, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the composition is formulated for ocular delivery.

In some embodiments, the composition further comprises, consists essentially of, or consists of an inhibitor of a biological activity of at least one molecule or complex selected from the group consisting of NLRC4, NLRP3, caspase-1, cyclic GMP-AMP synthase (cGAS), caspase-4, stimulator of interferon genes (STING), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR). In some embodiments, the inhibitor is a small interfering RNA (siRNA) or short hairpin RNA (shRNA) that targets a transcription product of a gene selected from the group consisting of NLRC4, NLRP3, caspase-1, cGAS, caspase-4, STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product. In some embodiments, the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

Accordingly, it is an object of the presently disclosed subject matter to provide compositions and methods for treating and/or preventing a disease, disorder, or condition associated with an NLR family CARD domain containing 4 (NLRC4) inflammasome biological activity.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIG. 1. SINE RNA induced NLRC4 phosphorylation (site S533) in mice macrophage (BMDM). Mouse bone marrow derived macrophage (BMDM) were treated with SINE RNAs (Alu (SEQ ID NO: 86), B1 (SEQ ID NO: 87), and B2 (SEQ ID NO: 88), each at 100 pmol), and polyI:C (10 μg/ml). Phosphorylated Nlrc4 (p-Nlrc4) and total Nlrc4 (t-Nlrc4) were detected by Western blot. Actin is provided as a loading control. The p-NLRC4 bands were indicated by black arrow.

FIG. 2. SINE RNA induced NLRC4 phosphorylation (site S533) through protein Kinase C Delta. BMDM cell pre-treated with the LRRK2 Kinase inhibitor gsk2578215a (GSK; 5-(2-Fluoro-4-pyridinyl)-2-(phenylmethoxy)-N-3-pyridinyl-benzamide; CAS Number 1285515-21-0; Sigma-Aldrich, St. Louis, Mo., United States of America) or the PKC delta inhibitor Rottlerin (CAS Number 82-08-6; Sigma-Aldrich) at 2 μM or 5 μM, and treated with Alu RNA (SEQ ID NO: 86) transfection or transfection reagent alone (Mock). NLRC4 phosphorylation (P-NLRC4) and supernatant caspase-1 activation were detected by SDS-PAGE. Results showed that Alu RNA (SEQ ID NO: 86) transfection induced NLRC4 phosphorylation and the cleavage of caspase-1 precursor (pro), which release the active form of caspase-1 (protein size is ˜20 kDa, labeled as p20) to cell medium (supernatant). PKC delta inhibitor, Rottlerin blocked Alu RNA-induced NLRC4 phosphorylation and caspase-1 activation. The p-NLRC4 and caspase-1 (p20) bands were indicated with black arrows.

FIGS. 3A and 3B. SINE RNA-induced ASC oligomerization is dependent on NLRC4 (BMDM). FIG. 3A. Immunofluorescence images of endogenous ASC specks and NLRC4 puncta in BMDMs after transfected with Alu (SEQ ID NO: 86) or B2 (SEQ ID NO: 88) RNA or transfection reagents only (Mock). The results showed that SINE RNA (Alu and B2, SEQ ID NOs: 86 and 88, respectively) induced the formation of ASC specks, which is the hallmark of inflammasome activation (Arrows indicated). Moreover, NLRC4 proteins assembled as puncta, and co-resided with ASC specks. FIG. 3B. ASC oligomerizations were evaluated in wild-type and Nlrc4^{-/-} BMDMs through cross-linking and western blot. The results showed that Alu RNA (SEQ ID NO: 86) induced ASC oligomerization was impaired in Nlrc4^{-/-} BMDMs. The bands of oligomeric ASC protein were indicated by black arrow.

FIG. 4. Alu RNA (SEQ ID NO: 86) induced NLRC4 protein oligomerization. Mouse BMDM was treated with Alu RNA (SEQ ID NO: 86; 100 pmol) at the indicated time points. NLRC4 oligomers were detected by Tris-glycine native PAGE. Loading control show by Actin. The results showed that Alu RNA (SEQ ID NO: 86) transfection induces the formation of NLRC4 oligomers, which indicates the assembly and activation of NLRC4 inflammasome induced by Alu RNA (SEQ ID NO: 86). The bands of oligomeric NLRC4 protein were indicated by black arrow.

FIGS. 5A and 5B. NLRC4 deficiency block Alu RNA (SEQ ID NO: 86) induced inflammasome (BMDM). FIG. 5A. Wild-type and Nlrc4^{-/-} BMDMs were transfected with Alu RNA (SEQ ID NO: 86), B2 RNA (SEQ ID NO: 88), poly(I:C) or mock. The expression of NLRC4, NLRP3, and Actin were detected by immunoblots with cell lysates. The cleavage of caspase-1 precursor (p45) and release of the active form of caspase-1 (p20) were measured by immunoblots with supernatant. The results showed that SINE RNA (Alu and B2, SEQ ID NOs: 86 and 88, respectively) induced caspase-1 cleavage was impaired in Nlrc4^{-/-} BMDMs. The bands of caspase-1 (p20) were indicated by black arrow. FIG. 5B. Wild-type and Nlrc4^{-/-} BMDMs were transfection with Alu (SEQ ID NO: 86), B2 RNA (SEQ ID NO: 88), poly(I:C), or mock. The IL-1β release was measured by ELISA. The results show that IL-1β release induced by SINE RNAs was blocked in Nlrc4^{-/-} BMDMs. Data show as Mean±SEM. **p<0.01; ***p<0.001.

FIGS. 6A and 6B. CLIP-Mass Spec identified Ddx5/Ddx17 potentially binds Alu RNA (SEQ ID NO: 86). FIG. 6A. Scatter plot of CLIP-LC-MS/MS for individual identified Alu RNA binding proteins. Quantitative analysis performed by Fisher's exact test on (median log₂(fold change)) of bait-specific protein enrichment (Biotinylated Alu RNA; SEQ ID NO: 86) in comparison to the background (Biotin) plotted against the corresponding −log₁₀(P value). The horizontal dotted line represents the log₂(fold change) cut-off and the vertical line represents that P value cut-off. FIG. 6B. Quantification of total spectra numbers enriched in Biotin-Alu RNA (SEQ ID NO: 86) samples. The enriched peaks of DDX5 and DDX17 were indicated by black arrows.

FIG. 7. Fluorescent staining shows co-localization of Ddx17 and Btn-Alu RNA (SEQ ID NO: 86) in human RPE cell. Human RPE cell treated with biotin-labeled Alu RNA (btn-Alu; SEQ ID NO: 86). Double staining on endogenous Ddx17 and btn-Alu RNA (SEQ ID NO: 86) show the co-colocalization between Ddx17 and Alu RNA (SEQ ID NO: 86). The results showed that Alu RNA (SEQ ID NO: 86) transfection induces nucleus-to-cytoplasm translocation of DDX17 proteins, and DDX17 colocalized with Alu RNA (SEQ ID NO: 86). The DDX17 and Alu RNA (SEQ ID NO: 86) dual positive signals were indicated by white arrows.

FIGS. 8A-8C. CLIP (cross-linking immunoprecipitation) on Alu RNA (SEQ ID NO: 86) and Ddx17 interaction. Human HEK293 cell transfected with btn-Alu RNA (SEQ ID NO: 86), cross-linked by ultraviolet light, pull-downed by biotin-streptavidin. The binding of Ddx17 determined by western blot. FIG. 8A. Schematic of CLIP (cross-linking immunoprecipitation) for biotin or myc-tag mediated immunoprecipitation. FIG. 8B. The results of streptavidin pull down (IP) of biotin-Alu RNA (SEQ ID NO: 86) show that DDX17 physically binds Alu RNA (SEQ ID NO: 86) in cells. WCL is whole cell lysates. The bands of pull downed DDX17 proteins by Alu RNA (SEQ ID NO: 86) were indicated by black arrow. FIG. 8C. The HEK293 cells were transfected with myc-tagged DDX17 and biotin-Alu RNA (SEQ ID NO: 86). Cells were cross-linked and lysates for immunoprecipitation by Anti-myc beads. The total RNA was extracted, and were the Alu RNA (SEQ ID NO: 86) were detected with Northern blot. The results showed that Alu RNA (SEQ ID NO: 86) interacted with DDX17 in cells. The bands of pull-downed Alu RNA (SEQ ID NO: 86) by myc-DDX17 proteins were indicated by black arrow.

FIG. 9. Alu RNA (SEQ ID NO: 86) induced co-localization of DDX17 and NLRC4 in BMDM cell. Wild type BMDM cell were treated with Alu RNA (SEQ ID NO: 86). Localization of endogenous Ddx17 and NLRC4 protein were detected by fluorescent staining. Results showed that Alu RNA (SEQ ID NO: 86) treatment induced the colocalization of DDX17 and NLRC4, which implies that Alu RNA (SEQ ID NO: 86) induced DDX17-NLRC4 assembly and NLRC4 inflammasome activation. The DDX17 and NLRC4 dual positive signals were indicated by white arrows.

FIG. 10. Co-Immunoprecipitation identified interaction of DDX17 and NLRC4 after Alu RNA (SEQ ID NO: 86) treatment. HEK293 cells transfected with flag-tagged NLRC4 plasmid and Alu RNA (SEQ ID NO: 86). Flag-immunoprecipitation were performed with various lysates. Results showed Alu RNA (SEQ ID NO: 86) treatment induced the interaction between NLRC4 and DDX17. The immunoprecipitated DDX17 and NLRC4 proteins were indicated by black arrows.

FIGS. 11A and 11B. Ddx17 involved in Alu RNA (SEQ ID NO: 86) induced inflammasome independent of its microprocessor function. DDX17 is a component of microprocessor complexes with DDX5 and Drosha. To test whether the microprocessor function of DDX17 is required for Alu RNA (SEQ ID NO: 86) induced inflammasome, we measured caspase-1 activation in THP cells with DDX5 or Drosha knockdown (siRNA for DDX5: GGAAAUUACAGUUAGAGGU; SEQ ID NO: 89); siRNA for Drosha: GACAAGUUGAUAGGAUAUA; SEQ ID NO: 90). FIG. 11A. siRNA mediated Ddx17 knockdown, but not Ddx5 in THP1 cells, blocked Alu RNA (SEQ ID NO: 86) induced caspase-1 activation. FIG. 11B. siRNA mediated Drosha knockdown did not affect Alu RNA (SEQ ID NO: 86) induced caspase-1 activation. The caspase-1 (p20) bands were indicated with black arrows.

FIGS. 12A and 12B. DDX17 knockdown blocks Alu RNA (SEQ ID NO: 86) induced ASC oligomerization and IL-1β release. FIG. 12A. siRNA mediated DDX17 knockdown (siRNA for DDX17: CCAAUCUGAUGUAUCAGGA; SEQ ID NO: 91), but not Ddx5 (siRNA for DDX5: GGAAAUUACAGUUAGAGGU; SEQ ID NO: 89) in THP1 cells, blocked Alu RNA (SEQ ID NO: 86) induced ASC oligomerization. The band of ASC oligomers were indicated by black arrow. FIG. 12B. siRNA mediated DDX17 knockdown, but not Ddx5 in THP1 cells, blocked Alu RNA (SEQ ID NO: 86) induced IL-1β release.

FIGS. 13A and 13B. Ddx17 deficiency blocks Alu RNA (SEQ ID NO: 86) induced inflammasome in BMDMs. Ddx17^{-/-} iBMDM cells blocked Alu RNA (SEQ ID NO: 86) induced caspase-1 activation (FIG. 13A) and IL-1β release (FIG. 13B). The caspase-1 (p20) bands were indicated with black arrow.

FIGS. 14A and 14B. Ddx17 knockdown does not affect Alu RNA (SEQ ID NO: 86) induced IFNI response and inflammatory priming. Wild type BMDM cell were transfected with siRNA target on DDX17 (CCAAUCUGAUGUAUCAGGA; SEQ ID NO: 91), and then 24 hours later, BMDM cells were treated with Alu RNA (SEQ ID NO: 86; 100 pmol). Total RNA extracted for qPCR assay. Results showed Ddx17 knockdown did not affect Alu RNA (SEQ ID NO: 86) induced type I interferon response (CXCL10, IFNB; FIG. 14A) or inflammatory priming (IL-1β (IL1b), CASPASE-1 (CASP1); FIG. 14B).

FIG. 15. DDX17 knockdown does not affect classical NLRC4 and NLRP3 inflammasome. THP1 cell were transfected with siRNA target on DDX17 (siRNA sequence: CCAAUCUGAUGUAUCAGGA; SEQ ID NO: 91), and then 24 hours later, Flagellin (3 μg/ml) or LPS (125 ng/ml) plus ATP (50 mM/30 minutes), DOTAP plus LPS was added to the THP1 cells. Supernatants were collected for Caspase-1 detection. Results showed that DDX17 knockdown with SEQ ID NO: 91 did not affect classical NLRC4 inflammasome and NLRP3 inflammasome. Loading control show by actin. The caspase-1 precursor is referred as p45, and the active form of caspase-1 as p20. The adaptor protein for inflammasome assembly, apoptosis-associated speck-like protein containing a CARD (ASC) is labeled as ASC. The caspase-1p20) bands were indicated with black arrow.

FIG. 16. Alu RNA (SEQ ID NO: 86) binding with DDX17 induces dual recruitment of NLRC4 and NLRP3. Alu RNA (SEQ ID NO: 86) treated wild type and Ddx17^{-/-} iBMDM cells were collected after 12 hours. Cell lysates were immunoprecipitated by NLRP3 antibody and immunoblotted with indicated antibodies. The bands of immunoprecipitated NLRC4 protein by NLRP3-IP were indicated with black arrow.

FIGS. 17A and 17B. NLRC4 deficiency blocks Alu RNA (SEQ ID NO: 86) induced NLRP3-ASC interaction (BMDM). FIG. 17A. Alu RNA (SEQ ID NO: 86) induced NLRP3-ASC interaction was abolished in Nlrc4^{-/-} BMDMs. The bands of immunoprecipitated NLRP3 protein by ASC-IP were indicated with black arrow. FIG. 17B. Alu RNA (SEQ ID NO: 86) induced ASC oligomerization was blocked in Nlrp3^{-/-} BMDMs. The bands of ASC oligomers are indicated by black arrow.

FIGS. 18A and 18B. NLRP3 deficiency blocks SINE RNA (SEQ ID NO: 86) induced NLRC4 inflammasome in BMDM. Alu RNA (SEQ ID NO: 86) induced caspase-1 activation (FIG. 18A) and IL1-β release (FIG. 18B) was impaired in Nlrp3^{-/-} BMDMs. ***p<0.001. The caspase-1 (p20) bands are indicated with black arrow.

FIGS. 19A and 19B. NAIP is dispensable for Alu RNA (SEQ ID NO: 86) induced NLRC4 inflammasome (BMDM). FIG. 19A. Flagellin-induced caspase-1 activation and IL-1β release were impaired in Naip^{-/-} BMDMs (Referred as Naip 1-6Δ/Δ). FIG. 19B. Alu RNA (SEQ ID NO: 86) induced caspase-1 cleavage and IL-1β release were not affected in Naip^{-/-} BMDMs. The caspase-1 (p20) bands are indicated with black arrows.

FIGS. 20A and 20B. DDX17-NLRC4-NLRP3 are required for Dicer1 knockdown induced inflammasome activation. FIG. 20A. Dicer1 knockdown (siRNA for Dicer 1: GCAGUUGUCCUAAACAGAU; SEQ ID NO: 92) causes increase of p-NLRC4 and caspase-1 activation, which were blocked in Ddx17^{-/-} iBMDMs. FIG. 20B. Dicer1 knockdown-induced caspase-1 cleavage was impaired in Nlrc4^{-/-} and Nlrp3^{-/-} BMDMs. The caspase-1 (p20) bands are indicated with black arrows.

FIGS. 21A and 21B. Expression level of Ddx17-Nlrc4 signaling in RPE tissue of dry AMD. FIG. 21A. Immunoblot on Ddx17, Nlrc4, PKCD with lysates of RPE tissue from Dry AMD patients showed significantly upregulated Ddx17 protein in dry AMD samples. FIG. 21B: Bar graph of the relative levels of the noted proteins from a scan of the immunoblot of FIG. 21A. The DDX17 bands are indicated with black arrow.

FIG. 22. DDX17 interacts with NLRC4 in the RPE of human donor eyes with dry AMD. To capture the interaction between DDX17 and NLRC4 in situ, we performed a proximity ligation assay (PLA) on human tissue sections from donor eyes with dry AMD or healthy controls. The results show that the DDX17 interacted with NLRC4 in the RPE of donor eye with dry AMD, which indicated the assembly of DDX17-NLRC4 complexes occurs in human dry AMD. The positive signal of DDX17-NLRC4 complexes are indicated by black arrows.

FIGS. 23A and 23B. Exogenous expression of NLRC4 hyperactive protein induce RPE degeneration. To test the consequence of NLRC4 activation in the RPE, 500 ng of plasmid encoding wild type (pNLRC4WT) and hyperactive mutant (pNLRC4T337S) of human NLRC4 protein were transduced into mouse RPE cell in vivo. Our results showed that NLRC4 activation caused RPE degeneration. FIG. 23A. Fundus images showed RPE degeneration induced by hyperactive NLRC4 protein. The hyperdense areas due to the RPE degeneration are indicated by white arrows. FIG. 23B. Fluorescent images showed disrupted ZO-1 staining in NLRC4T337S expressing cells. The RPE areas with ZO-1 disorganizations are indicated by white arrows.

FIGS. 24A and 24B. Alu RNA (SEQ ID NO: 86) induced NLRC4 activation in human RPE cells. To test whether Alu RNA (SEQ ID NO: 86) induces NLRC4 activation in human RPE, we transfected human RPE with Alu RNA (SEQ ID NO: 86 at 100 pmol). FIG. 24A. Immunofluorescence staining on NLRC4 show Alu RNA (SEQ ID NO: 86) induced cytosolic NLRC4 punctate in hRPE cells. The NLRC4 aggregates induced by Alu RNA (SEQ ID NO: 86) are indicated by white arrows. FIG. 24B. Alu RNA (SEQ ID NO: 86) induced NLRC4 (p-NLRC4) phosphorylation and oligomerization. Loading control show by Actin. NLRC4 oligomers were detected by Tris-glycine native PAGE. The data demonstrated that Alu RNA (SEQ ID NO: 86) induced NLRC4 activation in human RPE. The band of NLRC4 oligomers is indicated by black arrow.

FIGS. 25A and 25B. NLRC4 knockdown blocks Alu RNA (SEQ ID NO: 86) induced ASC oligomerization and RPE degeneration. Human RPE cell treated with siRNA target on NLRC4 (SMARTPOOL siRNA for NLRC4: CAACUGGGCUCCUCUGUAA; SEQ ID NO: 93) and NAIP (SMARTPOOL siRNA for NAIP: GUAAAGAGCUAUAUGGAUA; SEQ ID NO: 94), 24 hours later, treated with Alu RNA (SEQ ID NO: 86 at 100 pmol) FIG. 25A. Immunoblot show NLRC4, but not NAIP knockdown reduced Alu RNA (SEQ ID NO: 86) induced ASC oligomerization. The band of ASC oligomers was indicated by black arrow. FIG. 25B Fundus images and ZO-1 fluorescent images show NLRC4 knockdown blocked Alu RNA (SEQ ID NO: 86) induced RPE degeneration. The hyperdense areas due to the RPE degeneration in fundus images and ZO-1 disorganizations in RPE sheet are indicated by white arrows.

FIG. 26. Interfering DDX17-NLRC4 signaling blocks Alu RNA (SEQ ID NO: 86) induced RPE degeneration. Wilde type of C57/B6 mice were intravitreally injected with siRNA target on Ddx17 (siRNA for Ddx17: GGCUAGAUGUGGAAGAUGU; SEQ ID NO: 95), and two days later, Nlrc4^{-/-}, Naip1-6^{-/-}, mice and Ddx17 knockdown mice were subretinally injected with Alu RNA (SEQ ID NO: 86). Fundus images and ZO-1 fluorescent images showed that interfering with Ddx17 and Nlrc4, but not Naips, blocked Alu RNA (SEQ ID NO: 86) induced RPE degeneration. The hyperdense areas due to the RPE degeneration in fundus images and ZO-1 disorganizations in RPE sheet are indicated by white arrows.

FIG. 27. NRTI block Flagellin induced NLRC4 inflammasome in BMDM. Wild type BMDM cells were pre-treated with exemplary NRTIs (D4T, 3TC) at 100 μM for 1 hour, and then stimulated with flagellin transfection (3 μg/ml). Supernatant and cell lysate were collected for Caspase-1 detection. Results showed NRTI treatment reduced flagellin-induced caspase-1 activation. The caspase-1 (p20) bands are indicated with black arrow.

FIG. 28. NRTI (3TC) blocks flagellin-induced NLRC4 inflammasome in a dose dependent manner. Wild type BMDM cell were pre-treated with indicated dose of NRTI (3TC) for 1 hour, and then stimulated with Flagellin transfection (3 μg/ml). Supernatants were collected for Caspase-1 detection. Results showed that NRTI inhibited flagellin-induced Caspase-1 activation in a dose dependent manner. The caspase-1 (p20) bands are indicated with black arrow.

FIG. 29. NRTI (3TC) blocks flagellin induced NLRC4 Oligomerization in a dose dependent manner. Wild type BMDM cell were pre-treated with indicated dose of NRTI (3TC) for 1 hour, and then stimulated with Flagellin transfection (3 μg/ml). Cell pellets were collected for NLRC4 oligomers detection via Native Page electrophoresis. Results showed that the NRTI 3TC inhibited Flagellin induced NLRC4 oligomerization. The band of NLRC4 oligomers are indicated with black arrow.

FIG. 30. NRTI (3TC) blocks flagellin induced Interleukin 1 beta production. Wild type BMDM cell were pre-treated with indicated dose of NRTI (3TC) for 1 hour, and then stimulated with flagellin transfection (3 μg/ml). Supernatants were collected and assayed for secreted IL-1β. Results showed that flagellin induced the cleavage of IL-1β precursor (protein size is 30 kD; p30), and release of the active form of IL-1β (protein size is 17 kD: p17) into the cell medium (Sup). NRTI inhibited Flagellin induced IL-1β release. The bands of cleaved IL-1β are indicated with black arrow.

FIG. 31. Modified NRTIs (K8, K9) block flagellin-induced NLRC4 inflammasome in BMDM. Wild type BMDM cell were pre-treated with regular NRTI (D4T, 3TC), the modified NRTIs 3-Methyl-3TC (K9) or 2-Ethyl-AZT (K8), or NLRP3 inhibitors (MCC950, CY-09) for 1 hour, and then stimulated with flagellin transfection (3 μg/ml). Supernatants were collected for secreted caspase-1 detection. Results showed that modified NRTIs inhibited flagellin induced caspase-1 activation. The caspase-1 (p20) bands are indicated with black arrow.

FIG. 32. NRTIs block Flagellin-induced NLRC4 inflammasome in an NLRP3 dependent manner. NLRP3 knockout BMDM cell were pre-treated with regular NRTIs (D4T, 3TC) or NLRP3 inhibitors (MMC950, CY-09) for 1 hour, and then stimulated with flagellin transfection (3 μg/ml). Supernatants were collected for caspase-1 detection. Results showed that modified NRTIs did not inhibit flagellin induced caspase-1 activation in NLRP3 knockout BMDM. The caspase-1 (p20) bands are indicated with black arrow.

FIG. 33. NRTIs directly bind to NLRP3/NLRC4 complex in a reconstituted system. HEK293 cell were transfected with NLRC4 and NLRP3. Biotin-Labeled NRTIs (D4T, AZT) were added to the HEK cells 4 hours later, and cell pellets were collected for biotin streptavidin pulldown. Binding between NRTIs and NLRC4/NLRP3 was detected by immunoblot. Results showed that biotin-labeled NRTIs could directly bind to NLRC4 and NLRP3 protein. The bands of NLRC4 and NLRP3 pulled down with biotinylated NRTI are indicated with black arrows.

FIGS. 34A-34C. PKCδ and NLRC4 phosphorylation (S533) are required for Alu RNA (SEQ ID NO: 86) induced inflammasome activation. FIGS. 35A and 35B Wild-type, Prkcd^{-/+}, and Prkcd^{-/-} BMDMs were transfected with Alu RNA (SEQ ID NO: 86 at 100 pmol) for 12 hours. Supernatants were collected for measuring caspase-1, IL-1β cleavage, and IL-1β release. Cell lysates were collected for p-NLRC4, NLRC4, PKCδ, and actin blotting. Results showed that Caspase-1, IL-1β cleavage, and IL-1β release were impaired in Prkcd^{-/-} BMDMs. FIG. 35C. Wild-type and Nlrc4^{S533A/S533A} BMDMs were transfected with Alu RNA (SEQ ID NO: 86 at 100 pmol) for 12 hours. Supernatants were collected for measuring IL-1β release by ELISA. Results showed that IL-1β release was impaired in Nlrc4^{S533A/S533A} BMDMs. The bands of p-NLRC4 and caspase-1 (p20) are indicated by black arrows.

FIGS. 35A and 35B. PKCδ-mediated NLRC4 phosphorylation is required for Alu RNA (SEQ ID NO: 86) induced RPE degeneration. Wild-type, Prkcd^{-/-} and Nlrc4^{S533A/S533A} mice were subretinally injected with Alu RNA (SEQ ID NO: 86). Fundus images (FIG. 36A) and ZO-1 (FIG. 36B) flat mount fluorescent images showed that Alu RNA (SEQ ID NO: 86) induced RPE degeneration was blocked in Prkcd^{-/-} and Nlrc4^{S533A/S533A} mice. The hyperdense areas due to the RPE degeneration in fundus images and ZO-1 disorganizations in RPE sheet are indicated by white arrows.

FIG. 36. Alu RNA (SEQ ID NO: 86) induces DDX17 translocation in human cells. Human monocytes (THP-1) were transfected with Alu RNA (SEQ ID NO: 86 at 100 pmol) using LIPOFECTAMINE™ 3000 brand transfection reagent (Lipo; ThermoFisher Scientific). Cell lysates were collected and subjected to cell fractionation. Immunoblots of DDX17 and Histone H3 were used to evaluate the subcellular distribution of DDX17. Results show that Alu RNA (SEQ ID NO: 86) treatment induced DDX17 translocation from the nucleus to the cytoplasm, and DDX17 co-localized with cytosolic Alu RNA (SEQ ID NO: 86). The bands of DDX17 in cell nucleus and cytoplasm were indicated by black arrows.

FIG. 37. Alu RNA (SEQ ID NO: 86) induces the assembly of NLRC4 and NLRP3 complex. Human RPE cells were transfected with biotinylated Alu RNA (SEQ ID NO: 86 at 100 pmol) by LIPOFECTAMINE™ 3000 brand transfection reagent (Lipo; ThermoFisher Scientific). The assembly of NLRC4 and NLRP3 complex was evaluated by Proximity Ligation Assay (PLA). Results showed that Alu RNA (SEQ ID NO: 86) transfection induced the assembly of NLRC4 and NLRP3 complex in human RPE cells. The signal of NLRC4-NLRP3 complexes is indicated by the white arrow.

FIGS. 38A and 38B. The expression of DDX17 is increased in the RPE of human donor eyes with dry AMD. To measure DDX17 and NLRC4 expression in human eyes in situ, we detected the levels of DDX17 protein (FIG. 39A) and NLRC4 protein (FIG. 39B) in human donor eyes with dry AMD or healthy controls via immunohistochemistry. Results indicated that the expression of DDX17 was increased in the RPE of human donor eyes with dry AMD. The DDX17 and NLRC4 signals are indicated by black arrows.

### BRIEF DESCRIPTION OF THE SEQUENCE LISTING

SEQ ID NO: 1 is an exemplary nucleotide sequence of a human NLR family CARD domain containing 4 (NLRC4) gene product, and corresponds to Accession No. NM_021209.4 of the GENBANK® biosequence database.

SEQ ID NO: 2 is an amino acid sequence encoded by SEQ ID NO: 1, and corresponds to Accession No. NP_067032.3 of the GENBANK® biosequence database.

SEQ ID NOs: 3-6 are nucleotide sequences of exemplary siRNAs that target the nucleotide sequence of SEQ ID NO: 1 and other human NLRC4 gene products.

SEQ ID NO: 7 is an exemplary nucleotide sequence of a mouse NLR family CARD domain containing 4 (Nlrc4) gene product, and corresponds to Accession No. NM_001033367.3 of the GENBANK® biosequence database.

SEQ ID NO: 8 is an amino acid sequence encoded by SEQ ID NO: 7, and corresponds to Accession No. NP_001028539.1 of the GENBANK® biosequence database.

SEQ ID NOs: 9-20 are nucleotide sequences of exemplary siRNAs that target the nucleotide sequence of SEQ ID NO: 7 and other mouse Nlrc4 gene products.

SEQ ID NO: 21 is an exemplary nucleotide sequence of a human DEAD-box helicase 17 (DDX17) gene product, and corresponds to Accession No. NM_006386.5 of the GENBANK® biosequence database.

SEQ ID NO: 22 is an amino acid sequence encoded by SEQ ID NO: 21, and corresponds to Accession No. NP_006377.2 of the GENBANK® biosequence database.

SEQ ID NOs: 23-27 are nucleotide sequences of exemplary siRNAs that target the nucleotide sequence of SEQ ID NO: 21 and other human DDX17 gene products.

SEQ ID NO: 28 is an exemplary nucleotide sequence of a mouse DEAD-box helicase 17 (Ddx17) gene product, and corresponds to Accession No. NM_001040187.1 of the GENBANK® biosequence database.

SEQ ID NO: 29 is an amino acid sequence encoded by SEQ ID NO: 28, and corresponds to Accession No. NP_001035277.1 of the GENBANK® biosequence database.

SEQ ID NOs: 30-34 are nucleotide sequences of exemplary siRNAs that target the nucleotide sequence of SEQ ID NO: 28 and other mouse Ddx17 gene products.

SEQ ID NO: 35 is an exemplary nucleotide sequence of a human NLR family pyrin domain containing 3 (NLRP3) gene product, and corresponds to Accession No. NM_004895.5 of the GENBANK® biosequence database.

SEQ ID NO: 36 is an amino acid sequence encoded by SEQ ID NO: 35, and corresponds to Accession No. NP_004886.3 of the GENBANK® biosequence database.

SEQ ID NO: 37 is an exemplary nucleotide sequence of a mouse NLR family pyrin domain containing 3 (Nlrp3) gene product, and corresponds to Accession No. NM_001359638.1 of the GENBANK® biosequence database.

SEQ ID NO: 38 is an amino acid sequence encoded by SEQ ID NO: 37, and corresponds to Accession No. NP_001346567.1 of the GENBANK® biosequence database.

SEQ ID NO: 39 is an exemplary nucleotide sequence of a human caspase-1 (CASP1) gene product, and corresponds to Accession No. NM_033292.4 of the GENBANK® biosequence database.

SEQ ID NO: 40 is an amino acid sequence encoded by SEQ ID NO: 39, and corresponds to Accession No. NP_150634.1 of the GENBANK® biosequence database.

SEQ ID NO: 41 is an exemplary nucleotide sequence of a mouse caspase-1 (Casp1) gene product, and corresponds to Accession No. NM_009807.2 of the GENBANK® biosequence database.

SEQ ID NO: 42 is an amino acid sequence encoded by SEQ ID NO: 41, and corresponds to Accession No. NP_033937.2 of the GENBANK® biosequence database.

SEQ ID NO: 43 is an exemplary nucleotide sequence of a human caspase-4 (CASP4) gene product, and corresponds to Accession No. NM_001225.4 of the GENBANK® biosequence database.

SEQ ID NO: 44 is an amino acid sequence encoded by SEQ ID NO: 43, and corresponds to Accession No. NP_001216.1 of the GENBANK® biosequence database.

SEQ ID NO: 45 is a nucleotide sequence of an exemplary shRNA that targets the nucleotide sequence of SEQ ID NO: 43 and other human CASP4 gene products.

SEQ ID NOs: 46-51 are nucleotide sequences of exemplary siRNAs that target the nucleotide sequence of SEQ ID NO: 43 and other human CASP4 gene products.

SEQ ID NO: 52 is an exemplary nucleotide sequence of a mouse caspase-4 (Casp4) gene product, and corresponds to Accession No. NM_007609.3 of the GENBANK® biosequence database.

SEQ ID NO: 53 is an amino acid sequence encoded by SEQ ID NO: 52, and corresponds to Accession No. NP_031635.2 of the GENBANK® biosequence database.

SEQ ID NO: 54 is an exemplary nucleotide sequence of a human cyclic GMP-AMP synthase (CGAS) gene product, and corresponds to Accession No. NM_138441.3 of the GENBANK® biosequence database.

SEQ ID NO: 55 is an amino acid sequence encoded by SEQ ID NO: 54, and corresponds to Accession No. NP_612450.2 of the GENBANK® biosequence database.

SEQ ID NOs: 56 and 57 are nucleotide sequences of exemplary shRNAs that target the nucleotide sequence of SEQ ID NO: 54 and other human CGAS gene products.

SEQ ID NO: 58 is a nucleotide sequence of an exemplary siRNA that targets the nucleotide sequence of SEQ ID NO: 54 and other human CGAS gene products.

SEQ ID NO: 59 is an exemplary nucleotide sequence of a mouse cyclic GMP-AMP synthase (Cgas) gene product, and corresponds to Accession No. NM_173386.5 of the GENBANK® biosequence database.

SEQ ID NO: 60 is an amino acid sequence encoded by SEQ ID NO: 59, and corresponds to Accession No. NP_775562.2 of the GENBANK® biosequence database.

SEQ ID NO: 61 is an exemplary nucleotide sequence of a human stimulator of interferon response cGAMP interactor 1 (STING1) gene product, and corresponds to Accession No. NM_198282.4 of the GENBANK® biosequence database.

SEQ ID NO: 62 is an amino acid sequence encoded by SEQ ID NO: 61, and corresponds to Accession No. NP_938023.1 of the GENBANK® biosequence database.

SEQ ID NO: 63 is a nucleotide sequence of an exemplary shRNA that targets the nucleotide sequence of SEQ ID NO: 61 and other human STING1 gene products.

SEQ ID NO: 64 is an exemplary nucleotide sequence of a mouse stimulator of interferon response cGAMP interactor 1 (Sting1) gene product, and corresponds to Accession No. NM_028261.1 of the GENBANK® biosequence database.

SEQ ID NO: 65 is an amino acid sequence encoded by SEQ ID NO: 64, and corresponds to Accession No. NP_082537.1 of the GENBANK® biosequence database.

SEQ ID NO: 66 is an exemplary nucleotide sequence of a human peptidyl-prolyl cis-trans isomerase F (PPIF) gene product, and corresponds to Accession No. NM_005729.4 of the GENBANK® biosequence database.

SEQ ID NO: 67 is an amino acid sequence encoded by SEQ ID NO: 66, and corresponds to Accession No. NP_005720.1 of the GENBANK® biosequence database.

SEQ ID NO: 68 is a nucleotide sequence of an exemplary shRNA that targets the nucleotide sequence of SEQ ID NO: 66 and other human PPIF gene products.

SEQ ID NO: 69 is an exemplary nucleotide sequence of a mouse peptidyl-prolyl cis-trans isomerase F (Ppif) gene product, and corresponds to Accession No. NM_134084.1 of the GENBANK® biosequence database.

SEQ ID NO: 70 is an amino acid sequence encoded by SEQ ID NO: 69, and corresponds to Accession No. NP_598845.1 of the GENBANK® biosequence database.

SEQ ID NO: 71 is an exemplary nucleotide sequence of a human Gasdermin D (GSDMD) gene product, and corresponds to Accession No. NM_024736.7 of the GENBANK® biosequence database.

SEQ ID NO: 72 is an amino acid sequence encoded by SEQ ID NO: 71, and corresponds to Accession No. NP_079012.3 of the GENBANK® biosequence database.

SEQ ID NO: 73 is a nucleotide sequence of an exemplary shRNA that targets the nucleotide sequence of SEQ ID NO: 71 and other human GSDMD gene products.

SEQ ID NO: 74 is an exemplary nucleotide sequence of a mouse Gasdermin D (Gsdmd) gene product, and corresponds to Accession No. NM_026960.4 of the GENBANK® biosequence database.

SEQ ID NO: 75 is an amino acid sequence encoded by SEQ ID NO: 74, and corresponds to Accession No. NP_081236.1 of the GENBANK® biosequence database.

SEQ ID NO: 76 is an exemplary nucleotide sequence of a human interferon-beta (IFN-β) gene product, and corresponds to Accession No. NM_002176.4 of the GENBANK® biosequence database.

SEQ ID NO: 77 is an amino acid sequence encoded by SEQ ID NO: 76, and corresponds to Accession No. NP_002167.1 of the GENBANK® biosequence database.

SEQ ID NO: 78 is a nucleotide sequence of an exemplary shRNA that targets the nucleotide sequence of SEQ ID NO: 76 and other human IFN-β gene products.

SEQ ID NO: 79 is an exemplary nucleotide sequence of a mouse interferon-beta (Ifn-β) gene product, and corresponds to Accession No. NM_010510.1 of the GENBANK® biosequence database.

SEQ ID NO: 80 is an amino acid sequence encoded by SEQ ID NO: 79, and corresponds to Accession No. NP_034640.1 of the GENBANK® biosequence database.

SEQ ID NO: 81 is an exemplary nucleotide sequence of a human interferon-α/β receptor (IFNAR) gene product, and corresponds to Accession No. NM_001384498.1 of the GENBANK® biosequence database.

SEQ ID NO: 82 is an amino acid sequence encoded by SEQ ID NO: 81, and corresponds to Accession No. NP_001371427.1 of the GENBANK® biosequence database.

SEQ ID NO: 83 is a nucleotide sequence of an exemplary shRNA that targets the nucleotide sequence of SEQ ID NO: 81 and other human IFNAR gene products.

SEQ ID NO: 84 is an exemplary nucleotide sequence of a mouse interferon-α/β receptor (Ifnar) gene product, and corresponds to Accession No. NM_010508.2 of the GENBANK® biosequence database.

SEQ ID NO: 85 is an amino acid sequence encoded by SEQ ID NO: 84, and corresponds to Accession No. NP_034638.2 of the GENBANK® biosequence database.

SEQ ID NO: 86 is a nucleotide sequence for an exemplary Alu RNA.

SEQ ID NO: 87 is a nucleotide sequence for an exemplary B1 RNA.

SEQ ID NO: 88 is a nucleotide sequence for an exemplary B2 RNA.

SEQ ID NO: 89 is a nucleotide sequence for an exemplary siRNA targeted to a DDX5 gene product.

SEQ ID NO: 90 is a nucleotide sequence for an exemplary siRNA targeted to a Drosha gene product.

SEQ ID NO: 91 is a nucleotide sequence for an exemplary siRNA targeted to a DDX17 gene product.

SEQ ID NO: 92 is a nucleotide sequence for an exemplary siRNA targeted to a Dicer 1 gene product.

SEQ ID NO: 93 is a nucleotide sequence for an exemplary siRNA targeted to an NLRC4 gene product.

SEQ ID NO: 94 is a nucleotide sequence for an exemplary siRNA targeted to an NAIP gene product.

SEQ ID NO: 95 is a nucleotide sequence for an exemplary siRNA targeted to a Ddx17 gene product.

### DETAILED DESCRIPTION

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, a subject is a human.

It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals. By way of example and not limitation, the GENBANK® biosequence database includes Accession No. NM_021209.4 corresponding to nucleotide sequences of a human NLRC4 gene product, and NM_001033367.3 corresponding to the nucleotide sequence of a mouse Nlrc4 gene product, among others, and Accession No. NM_006386.5 corresponding to the nucleotide sequences of a human DDX17 gene product and NM_001040187.1 corresponding to the nucleotide sequence of a mouse Ddx17 gene product, among others. It is understood that the term “Nlrc4” refers to NLR family CARD domain containing 4 genes and gene products from other animals and variants thereof, and the term “Ddx17” refers to DEAD-box helicase 17 (Ddx17) genes and gene products from other animals and variants thereof.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, hypertension and diabetes. The additional compounds can also be used to treat symptoms associated with the injury, disease, or disorder, including, but not limited to, pain and inflammation.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe peptide compounds as disclosed herein follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990 modified as in Karlin & Altschul, 1993. This algorithm is incorporated into the NBLAST and XBLAST programs (see Altschul et al., 1990a; Altschul et al., 1990b, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein, the phrase “inhibitory nucleic acid” refers to any nucleic acid molecule capable of mediating RNA interference (RNAi) or gene silencing. See e.g., Bass, 2001; Elbashir et al., 2001; and PCT International Publication Nos. WO 99/07409; WO 99/32619; WO 00/01846; WO 00/44895; WO 00/44914; WO 01/36646; and WO 01/29058. Exemplary inhibitory nucleic acids include small interfering RNAs, short interfering RNAs, siRNAs, and miRNAs. In some embodiments, the inhibitory nucleic acid comprises a double stranded polynucleotide molecule comprising complementary sense and antisense regions, wherein the antisense region comprises a sequence complementary to a region of a target nucleic acid molecule. For example, in some embodiments the inhibitory nucleic acid comprises, consists essentially of, or consists of an antisense region complementary to a region of a transcription product of a gene selected from the group consisting of NLRC4, NLRP3, DDX17, caspase-1, caspase-4, cGAS, STING1, PPIF, MPTP, GSDMD, IFN-β, and IFNAR; optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84. In some embodiments, the inhibitory nucleic acid comprises a single stranded polynucleotide having self-complementary sense and antisense regions, wherein the antisense region comprises a sequence complementary to a region of a target nucleic acid molecule. In some embodiments, the inhibitory nucleic acid comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active inhibitory nucleic acid capable of mediating RNAi. In some embodiments, the inhibitory nucleic acid is an siRNA, which in some embodiments comprises, consists essentially of, or consists of a nucleotide sequence as set forth in any of SEQ ID NOs: 3-6 (hNLRC4 siRNAs), 9-20 (mNlrc4 siRNAs), 23-27 (hDDX17 siRNAs), 30-34 (mDdx17 siRNAs), 46-51 (hCAS-4 siRNAs), and 58 (hCGAS siRNA). In some embodiments, the inhibitory nucleic acid is an shRNA, which in some embodiments comprises, consists essentially of, or consists of a nucleotide sequence as set forth in any of SEQ ID NO: 45 (hCAS-4 shRNA), 56 (hCGAS shRNA), 57 (hCGAS shRNA), 63 (hSTING1 shRNA), 68 (hPPIF shRNA), 73 (hGSDMD shRNA), 78 (hIFN-β shRNA), and 83 (hIFNAR shRNA).

As used herein, inhibitory nucleic acid molecules need not be limited to those molecules containing only RNA, but further encompass chemically modified nucleotides and non-nucleotides.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal routes. In some embodiments, the composition is formulated for ocular delivery.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to cells, refers to a single cell of interest, or a population of cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than cells of interest. Purity can be measured by any appropriate method, such as but not limited to those presented in the EXAMPLES.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like that has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Compositions

In some embodiments, the presently disclosed subject matter relates to compositions for use in the methods disclosed herein, including but not limited to the methods for treating and/or preventing a disease, disorder, and/or condition associated with an NLRC4 inflammasome biological activity, for inhibiting NLRC4-induced caspase-1 activation in cells, for inhibiting NLRC4-induced IL-1β release from cells, and for inhibiting Alu-induced retinal pigmented cell (RPE) degeneration in subjects.

Accordingly, in some embodiments the presently disclosed subject matter provides compositions for use in treating and/or preventing a disease, disorder, or condition associated with an NLRC4 inflammasome biological activity.

In some embodiments, the presently disclosed subject matter provides compositions for use in inhibiting NLRC4-induced IL-1β release from cells.

In some embodiments, the presently disclosed subject matter provides compositions for use in inhibiting Alu-induced retinal pigmented cell (RPE) degeneration in subjects.

In some embodiments, the compositions of the presently disclosed subject matter comprise, consist essentially of, or consist of one or more nucleoside reverse transcriptase inhibitors (NRTIs). A multitude ofNRTIs are known, and include but are not limited to the following: abacavir ((1S,4R)-4-[2-amino-6-(cyclopropylamino)-9H-purin-9-yl]cyclopent-2-en-1-yl}methanol; ABC; U.S. Pat. No. 8,183,370), adefovir ({[2-(6-amino-9H-purin-9-yl)ethoxy]methyl}phosphonic acid; bis-POM PMEA; U.S. Pat. No. 5,663,159), amdoxovir ([(2R,4R)-4-(2,6-diaminopurin-9-yl)-1,3-dioxolan-2-yl]methanol; Murphy et al., 2010), apricitabine (4-amino-1-[(2R,4R)-2-(hydroxymethyl)-1,3-oxathiolan-4-yl]pyrimidin-2(1H)-one; AVX754; PCT International Patent Application Publication No. WO 2014/183147), censavudine (1-[(2R,5R)-5-ethynyl-5-(hydroxymethyl)-2H-furan-2-yl]-5-methylpyrimidine-2,4-dione; U.S. Pat. Nos. 7,589,078; 8,193,165; 9,126,971), didanosine (9-((2R,5S)-5-(hydroxymethyl)tetrahydrofuran-2-yl)-3H-purin-6(9H)-one; DDI; U.S. Pat. Nos. 7,589,078; 8,193,165; 9,126,971), elvucitabine (4-amino-5-fluoro-1-[(2S,5R)-5-(hydroxymethyl)-2,5-dihydrofuran-2-yl]pyrimidin-2-one; U.S. Patent Application Publication No. 2011/0150997), emtricitabine (2′,3′-dideoxy-5-fluoro-3′-thiacytidine 4-amino-5-fluoro-1-[(2R,5S)-2-(hydroxymethyl)-1,3-oxathiolan-5-yl]-1,2-dihydropyrimidin-2-one; FTC; PCT International Patent Application Publication No. WO 2014/176532), entecavir (2-Amino-9-[(1S,3R,4S)-4-hydroxy-3-(hydroxymethyl)-2-methyl-idenecyclopentyl]-1H-purin-6-one; ETV; U.S. Pat. No. 6,627,224), lamivudine (2′,3′-dideoxy-3′-thiacytidine-4-Amino-1-[(2R,5S)-2-(hydroxymethyl)-1,3-oxathiolan-5-yl]-1,2-dihydropyrimidin-2-one; 3TC; U.S. Pat. No. 8,481,554), racivir (4-amino-5-fluoro-1-[(2S,5R)-2-(hydroxymethyl)-1,3-oxathiolan-5-yl]-1,2-dihydropyrimidin-2-one; Otto, 2004, stampidine (methyl N-((4-bromophenoxy){[(2S,5R)-5-(5-methyl-2,4-dioxo-3,4-dihydropyrimidin-1(2H)-yl)-2,5-dihydrofuran-2-yl]methoxy}phosphoryl)-D-alaninate; U.S. Pat. No. 6,350,736), stavudine (1-[(2R,5S)-5-(hydroxymethyl)-2,5-dihydrofuran-2-yl]-5-methyl-1,2,3,4-tetrahydropyrimidine-2,4-dion; d4T; U.S. Pat. No. 8,026,356), tenofovir disoproxil (Bis{[(isopropoxycarbonyl)oxy]methyl}({[(2R)-1-(6-amino-9H-purin-9-yl)-2-propanyl]oxy}methyl)phosphonate; TDF; PCT International Patent Application Publication No. WO 2008/007382), tenofovir alafenamide (Isopropyl (2S)-2-[[[(1R)-2-(6-aminopurin-9-yl)-1-methyl-ethoxy]methyl-phenoxy-phosphoryl]amino]propanoate; GS-7340; U.S. Pat. No. 9,296,769), zalcitabine (4-amino-1-((2R,5S)-5-(hydroxymethyl)tetrahydrofuran-2-yl)pyrimidin-2(1H)-one; ddC; Shelton et al., 1993), zidovudine (ZDV)/azidothymidine (3′-deoxy-3′-azidothymidine 1-[(2R,4S,5S)-4-Azido-5-(hydroxymethyl)oxolan-2-yl]-5-methylpyrimidine-2,4-dione; AZT; U.S. Pat. Nos. 5,905,082; 6,294,540; 6,417,191), derivatives thereof, optionally alkylated derivatives thereof, further optionally tri-methoxy-3TC (also known as Kamuvudine-9 and K-9), pharmaceutically acceptable salts thereof, and combinations thereof. See also U.S. Patent Application Publication Nos. 2019/0022115, 2019/0055273, 2019/0177326, 2019/0185508. Each of these U.S. patents, U.S. Patent Applications Publications, and PCT International Patent Applications Publications is incorporated by reference in its entirety. Thus, in some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

In some embodiments, a composition of the presently disclosed subject matter is prepared as a pharmaceutical composition. Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal routes.

The presently disclosed subject matter is also directed to pharmaceutical compositions comprising the compositions of the presently disclosed subject matter. More particularly, such compounds can be formulated as pharmaceutical compositions using standard pharmaceutically acceptable carriers, fillers, solubalizing agents, and stabilizers known to those skilled in the art.

The presently disclosed subject matter also encompasses the use pharmaceutical compositions of an appropriate compound, homolog, fragment, analog, or derivative thereof to practice the methods disclosed herein, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

The presently disclosed subject matter also encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, and mammals including commercially relevant mammals such as cattle, pigs, horses, sheep, cats, and dogs.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. In some embodiments, the composition is formulated for ocular delivery. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient(s), the pharmaceutically acceptable carrier(s), and any additional ingredients in a pharmaceutical compositions of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical compositions of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for oral and/or nasal administration may, for example, comprise from about as little as 0.10% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences, Genaro (ed.) (1985) Mack Publishing Co., Easton, Pa., United States of America, which is incorporated herein by reference in its entirety.

Typically, dosages of the compound of the presently disclosed subject matter which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In one embodiment, the dosage of the compound will vary from about 10 μg to about 10 g per kilogram of body weight of the animal. In another embodiment, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

In some embodiments, the compositions of the presently disclosed subject matter include additional therapeutic agents, which in some embodiments comprise, consist essentially of, or consist of an inhibitor of a biological activity of at least one molecule or complex selected from the group consisting of NLRC4, NLRP3, caspase-1, cyclic GMP-AMP synthase (cGAS), caspase-4, stimulator of interferon genes (STING), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR). In some embodiments, the inhibitor is a small interfering RNA (siRNA) or short hairpin RNA (shRNA) that targets a transcription product of a gene selected from the group consisting of NLRC4, NLRP3, caspase-1 (CAS-1), cGAS, caspase-4 (CAS-4), STING, PPIF, MPTP, GSDMD, IFN-β, and IFNAR. In some embodiments, the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR. Nucleic acid- and antibody-based inhibitors of NLRC4, NLRP3, CAS-1, cyclic CGAS, CAS-4, STING, PPIF, MPTP, GSDMD, IFN-0, and IFNAR are disclosed, for example in PCT International Patent Application Publication No. WO 2019/074884, which is incorporated herein by reference in its entirety.

### III. Methods of Use and Treatment

As disclosed herein, the cellular sensor that recognizes SINE RNAs which activate the inflammasome has been identified as Ddx17. Unexpectedly, upon binding with SINE RNAs, Ddx17 causes dual recruitment of NLRC4, NLRP3 protein, as well as apoptosis associated speck-like protein containing a CARD (ASC) protein, followed by the downstream inflammatory cascade. Moreover, SINEs activated the NLRC4 inflammasome independent of NAIP, which is required for classical NLRC4 inflammasome upon bacterial infection. Our data suggest that Ddx17-NLRC4-NLRP3 signaling contributes to RPE degeneration, a clinical and pathological hallmark of geographic atrophy, an advanced form of AMD. Collectively, our study provides novel insights into the first report of a sterile non-canonical NLRC4 inflammasome pathway activated by SINEs independent of NAIPs and also describes an unexpected role of NLRC4 inflammasome in the pathology of AMD.

Accordingly, in some embodiments of the presently disclosed subject matter, SINE RNAs, which have been implicated in multiple diseases such as but not limited to macular degeneration, Alzheimer's disease, lupus, etc., have been found to activate the NLFC4 inflammasome in a previously unknown manner. More particularly, SINE RNAs are recognized by DDX17, which interacts with NLRC4 and provides the activation of NLRC4 independent of NAIPs, which were previously thought to be required for NLRC4 activation. As further described herein, siRNAs targeting DDX17 or NLRC4 block cellular inflammation and cell death, including retinal degeneration. NRTIs or alkylated NRTIs also block NLRC4 activation, and thus represent the first small molecule inhibitors of NLRC4 and drug candidates for multiple diseases.

Therefore, in some embodiments the presently disclosed subject matter relates to methods for treating and/or preventing diseases, disorders, and/or conditions associated with NLRC4 inflammasome biological activity. As used herein, the phrase “diseases, disorders, and/or conditions associated with NLRC4 inflammasome biological activity” refer to any disease, disorder, and/or condition at least one symptom of which results from NLRC5 biological activity, either directly or indirectly, and for which an improvement in a cell and/or in a subject can result from treatment of the cell and/or the subject with the compositions and methods of the presently disclosed subject matter. Exemplary diseases, disorders, and/or conditions associated with NLRC4 inflammasome biological activity include, but are not limited to graft-versus-host disease, chronic pain, proliferative vitreoretinopathy, glaucoma, rheumatoid arthritis, multiple sclerosis, bipolar disorder, major depressive disorder, renal fibrosis, nephritis, pulmonary fibrosis, Huntington's disease, osteoporosis, chronic lymphocytic leukemia, anxiety disorders, pulmonary tuberculosis, osteoporosis in post-menopausal women and fracture patients, systemic lupus erythematosus, chronic inflammatory and neuropathic pain, autosomal dominant polycystic kidney disease, spinal cord injury, Alzheimer's disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson's disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., Leishmania spp, SARS-CoV, SARS-CoV-2 and Chlamydia spp., cryopyrinopathies; keratitis, acne vulgaris, Crohn's disease, ulcerative colitis, irritable bowel syndrome, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, chronic obstructive pulmonary disease, drug-induced lung inflammation, contact dermatitis, leprosy, Burkholderia cenocepacia infection, respiratory syncytial virus infection, psoriasis, scleroderma, reactive arthritis, cystic fibrosis, syphilis, Sjogren's syndrome, inflammatory joint disease, non-alcoholic fatty liver disease, cardiac surgery (peri-/post-operative inflammation), acute and chronic organ transplant rejection, acute and chronic bone marrow transplant rejection, and tumor angiogenesis. A particular diseases, disorders, and/or conditions associated with NLRC4 inflammasome biological activity is age-related macular degeneration (AMD) and/or geographic atrophy.

In some embodiments of the presently disclosed subject matter, the methods for treating and/or preventing diseases, disorders, and/or conditions associated with NLRC4 inflammasome biological activity comprise, consist essentially of, or consist of administering to a subject in need thereof a composition comprising, consisting essentially of, or consisting of an NRTI, wherein the administering is via an route and in an amount effective for reducing the NLRC4 inflammasome biological activity, thereby treating and/or preventing the disease, disorder, or condition associated with the NLRC4 inflammasome biological activity. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

In particular embodiments of the presently disclosed subject matter, the disease, disorder, and/or condition associated with the NLRC4 inflammasome biological activity is a disease of the retinal pigmented epithelium (RPE), which in some embodiments can include age-related macular degeneration (AMD) and/or geographic atrophy.

Similarly, in some embodiments the presently disclosed subject matter relates to methods for inhibiting NLRC4-induced caspase-1 activation in cells. In some embodiments, the methods comprise, consist essentially of, or consist of contacting an NLRC4 gene product and/or a complex of an NLRC4 gene product and an NLRP3 gene product with an effective amount of a composition comprising, consisting essentially of, or consisting of an NRTI, whereby NLRC4-induced caspase-1 activation is inhibited in the cell. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

In some embodiments, of the presently disclosed methods, the cell is present in a subject, optionally a mammalian subject, further optionally a human subject.

Additionally, in some embodiments the presently disclosed subject matter relates to methods for inhibiting NLRC4-induced IL-1 release from a cell, which in some embodiments can comprise, consist essentially of, or consist of contacting an NLRC4 gene product and/or a complex of an NLRC4 gene product and an NLRP3 gene product with an effective amount of a composition comprising, consisting essentially of, or consisting of a nucleoside reverse transcriptase inhibitor (NRTI), whereby NLRC4-induced IL-1β release from the cell is inhibited. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

In some embodiments, of the presently disclosed methods, the cell is present in a subject, optionally a mammalian subject, further optionally a human subject.

In some embodiments, the NLRC4-induced caspase-1 activation and/or the NLRC4-induced IL-1β release is associated with a disease, disorder, and/or condition associated with an NLR family CARD domain containing 4 (NLRC4) inflammasome biological activity. In some embodiments, the disease, disorder, and/or condition associated with the NLRC4 inflammasome biological activity is a disease of the retinal pigmented epithelium (RPE), optionally age-related macular degeneration (AMD) and/or geographic atrophy.

Additionally, in some embodiments the presently disclosed subject matter relates to methods for inhibiting Alu-induced retinal pigmented cell (RPE) degeneration in subjects, which in some embodiments can comprise, consist essentially of, or consist of contacting an NLRC4 gene product and/or a complex of an NLRC4 gene product and an NLRP3 gene product in a cell of the subject with an effective amount of a composition comprising, consisting essentially of, or consisting of a nucleoside reverse transcriptase inhibitor (NRTI), whereby NLRC4-induced IL-1β release from the cell is inhibited. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the cell is an RPE cell that present in a subject, optionally a mammalian subject, further optionally a human subject. In some embodiments, the presently disclosed methods further comprise, consist essentially of, or consist of administering to the subject at least one additional treatment designed to protect the RPE from degradation.

Thus, and as would be understood by one of ordinary skill in the art, in some embodiments the compositions and methods of the presently disclosed subject matter are part of a combination therapy, wherein appropriate therapies other than NRTI treatment are employed, depending on the disease, disorder, and/or condition to be treated. By way of example and not limitation, in some embodiments the presently disclosed methods further comprise, consist essentially of, or consist of administering to the subject in need thereof at least one additional inhibitor of the NLRC4 inflammasome biological activity. In some embodiments, the at least one additional inhibitor is selected from the group consisting of an antisense oligonucleotide, a small interfering RNA (siRNA), a short hairpin RNA (shRNA), and an antibody or antigen-binding fragment thereof siRNAs and shRNAs that can be employed in the compositions and methods of the presently disclosed subject matter are disclosed herein above, and include nucleic acids that that target a transcription product of a gene selected from the group consisting of NLRC4, NLRP3, caspase-1 (CAS-1), CGAS, caspase-4 (CAS-4), STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

In some embodiments, the at least one additional treatment comprises administering to the subject an inhibitor of a biological activity of at least one molecule or complex selected from the group consisting of NLRC4, NLRP3, CAS-1, CGAS, CAS-4, STING, PPIF, MPTP, GSDMD, IFN-β, and IFNAR. In some embodiments, the inhibitor is a small interfering RNA (siRNA) or short hairpin RNA (shRNA) that targets a transcription product of a gene selected from the group consisting of NLRC4, NLRP3, CAS-1, cGAS, CAS-4, STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-0 transcription product, and SEQ ID NO: 83 and targets a human IFNAR transcription product.

In some embodiments, the inhibitor is an antibody or antigen-binding fragment thereof that binds to a translation product of a gene selected from the group consisting of NLRC4, NLRP3, CAS-1, cGAS, CAS-4, STING, PPIF, MPTP, GSDMD, IFN-β, and IFNAR. Antibodies and fragments thereof that bind to NLRC4, NLRP3, CAS-1, cGAS, CAS-4, STING, PPIF, MPTP, GSDMD, IFN-β, and IFNAR gene products can be easily produced using methods known in the art (see e.g., Harlan & Lane, 1988). Alternatively or in addition, antibodies and antigen-binding fragments thereof that bind to these gene products are commercially available from sources including, but not limited to Abcam (Cambridge, United Kingdom), Santa Cruz Biotechnology, Inc. (Santa Cruz, Calif., United States of America), Sigma-Aldrich (St. Louis, Mo., United States of America), and others.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Example 1

### SINE RNAs Induce NLRC4 Inflammasome Activation

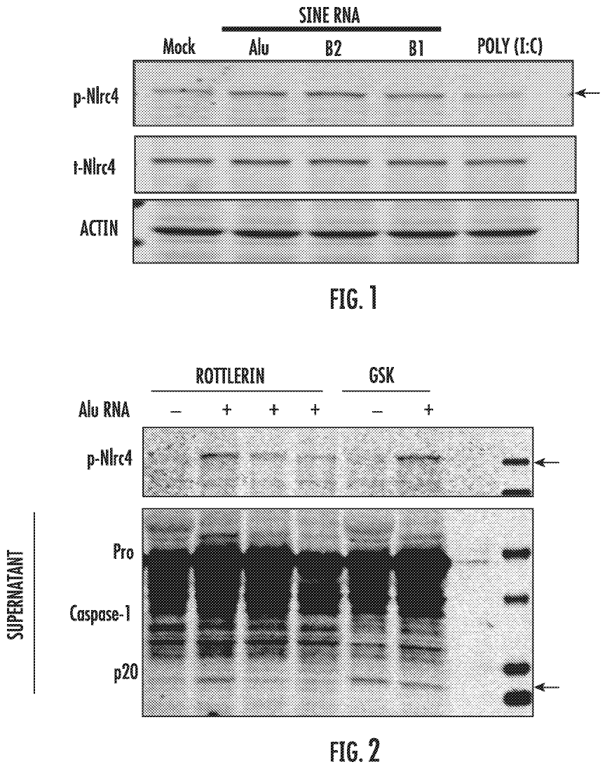

Previous studies have shown that NLRC4 inflammasome activation in response to Salmonella exposure requires two steps for its activation, phosphorylation on Ser533 residue of NLRC4 and NAIP mediated oligomerization of NLRC4. Immunoblotting revealed that human Alu RNA (SEQ ID NO: 86) as well as mouse B1 (SEQ ID NO: 87) and B2 (SEQ ID NO: 88) RNAs induced NLRC4 phosphorylation on S533 in BMDMs. Notably, the long dsRNA mimetic poly(I:C) did not induce NLRC4 phosphorylation (FIG. 1). Moreover, treatment with SINE RNAs induced caspase-1 p20 cleavage, indicative of inflammasome activation (FIG. 2). For the subsequent experiments in this study, we used Alu RNA (SEQ ID NO: 86) as the stimulus to activate the NLRC4 inflammasome. To date, two kinases are reported to phosphorylate NLRC4 at S533: protein kinase C delta (PKCδ) and leucine rich repeat-containing kinase-2 (LRRK2). To explore the roles of these kinases in Alu RNA (SEQ ID NO: 86) induced NLRC4 phosphorylation, we pretreated BMDMs with pharmacological inhibitors of LRRK2 (GSK2578215A, GSK) and PKCδ (Rottlerin, Rot) respectively. Rottlerin, but not GSK, inhibited Alu RNA (SEQ ID NO: 86) induced phosphorylation of NLRC4 and caspase-1 activation in BMDMs (FIG. 2), suggesting that PKCδ and not LRRK2 is involved in this process.

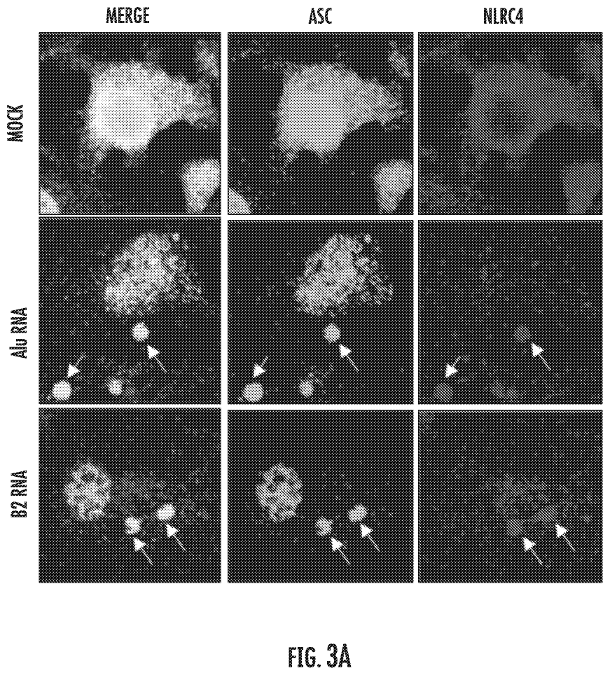

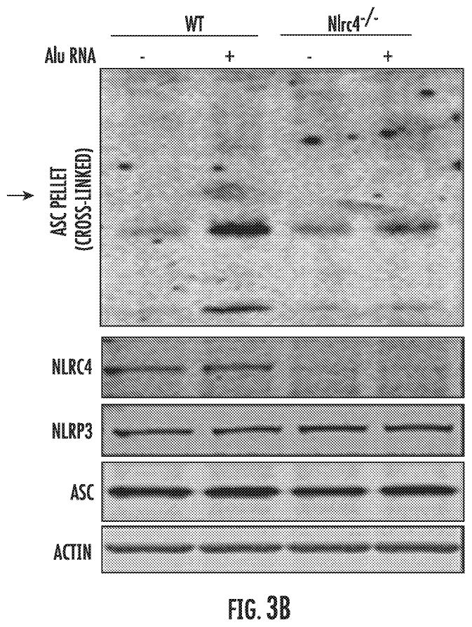

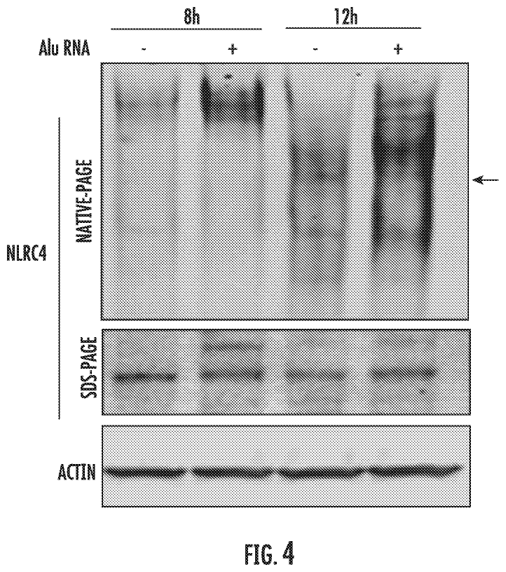

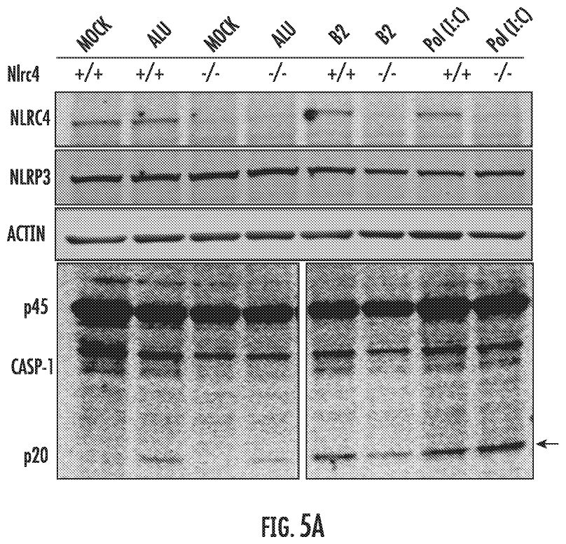

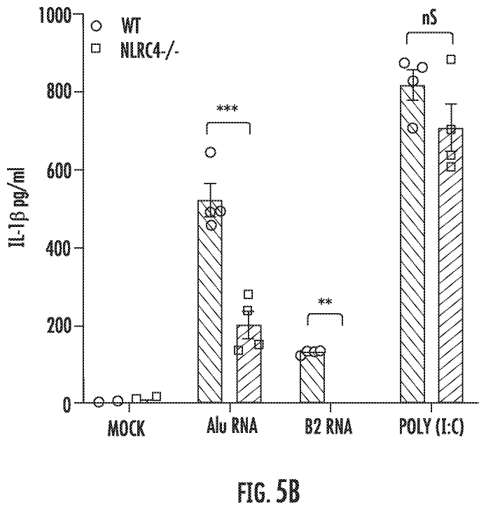

The activated NLRC4 inflammasome undergoes oligomerization by assembling into high-molecular mass multiprotein complexes. We first investigated in situ assembling of NLRC4 and apoptosis speck (ASC) like protein complexes in response to SINE RNA treatment of BMDMs. Immunofluorescence studies show increased NLRC4 and ASC specks in SINE RNA-treated BMDMs compared to mock-treated cells (FIG. 3A). To assess NLRC4 inflammasome assembly, WT and NLRC4 KO BMDM cells were treated with Alu RNA (SEQ ID NO: 86). Disuccinimidyl suberate (DSS) cross-linked lysates were resolved using denaturing SDS-PAGE to detect ASC oligomers and cell lysates were resolved using native polyacrylamide gel electrophoresis (NATIVE-PAGE) to detect NLRC4 oligomerization. Immunoblotting revealed that Alu RNA (SEQ ID NO: 86) induced the formation of ASC oligomers is dependent on NLRC4 (FIG. 3B) as well as typical large oligomeric NLRC4 complexes induced by SINE RNA (FIG. 4). NLRC4/ASC oligomerization is essential for the cleavage of pro-caspase-1 into its active form p20 and IL-1β release. Significantly, activation of caspase-1 and IL-1β release induced by SINE RNA were impaired in NLRC4 BMDMs (FIGS. 5A and 5B). Overall, these data demonstrate that SINE RNA induces NLRC4 inflammasome activation in mouse BMDMs, as monitored by phosphorylation, oligomerization and ELISA assays, and suggest PKCδ is the kinase responsible for NLRC4 phosphorylation.

### Example 2

### DDX17 is Required for SINE RNA-Induced NLRC4 Inflammasome Activation

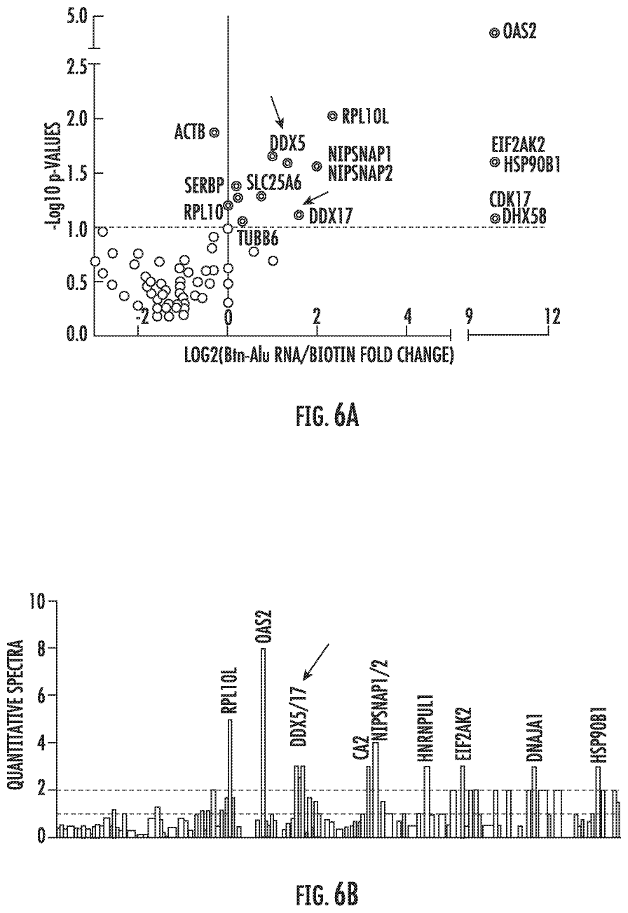

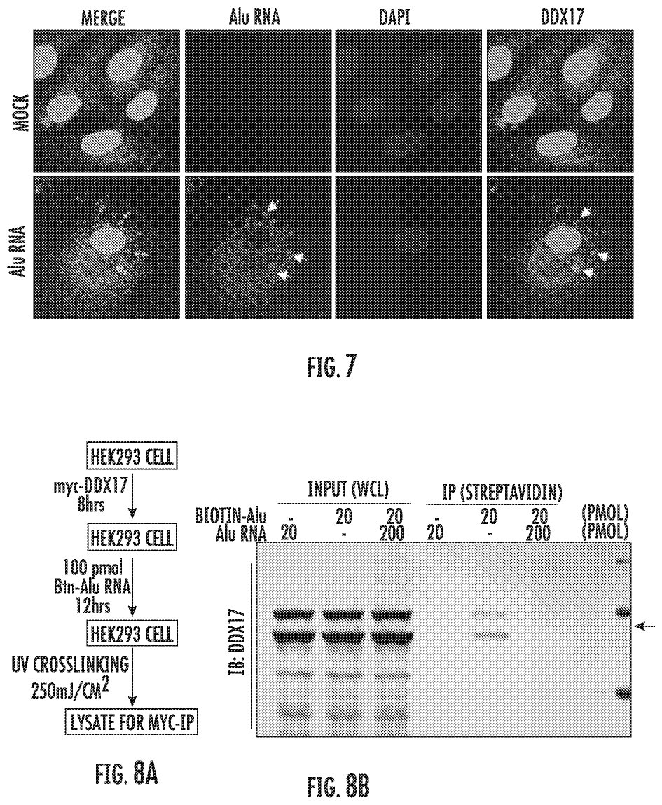

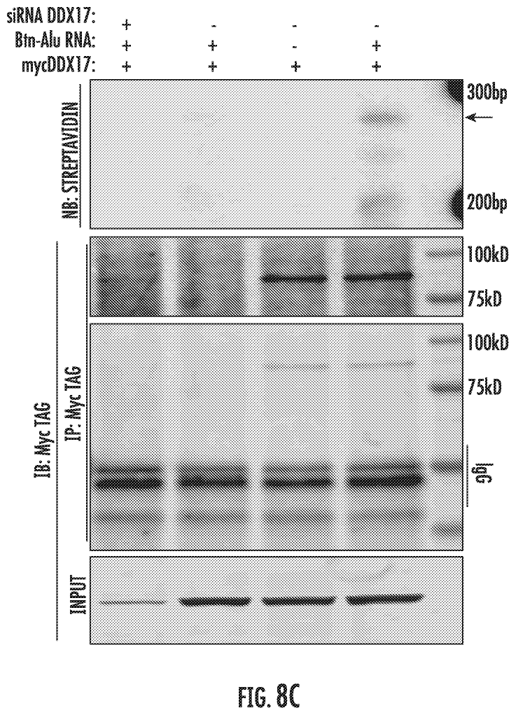

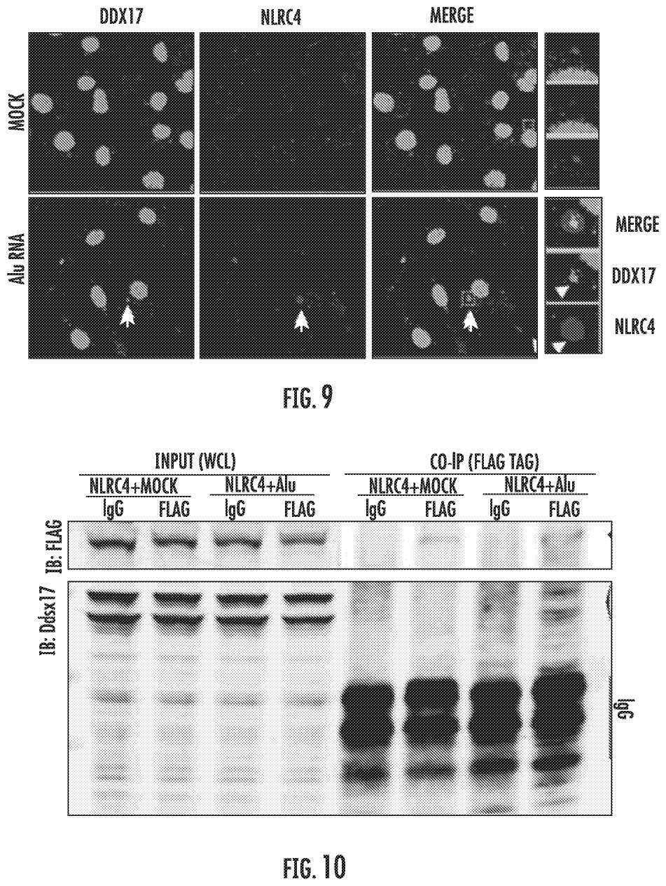

Next, we sought to identify the sensor responsible for Alu RNA (SEQ ID NO: 86) induced NLRC4 inflammasome activation. We employed cross-linking immunoprecipitation (CLIP)-mass spectrometry to reveal protein interaction partners of Alu RNA (SEQ ID NO: 86). Subsequently, DDX5 and DDX17 were identified as potential Alu RNA interacting partners (FIGS. 6A and 6B). Immunolocalization studies show superimposition of biotin-labeled Alu RNA (SEQ ID NO: 86) and DDX17 in WT mouse BMDMs (FIG. 7). Next, we investigated whether DDX17 interacted with Alu RNA (SEQ ID NO: 86) using a CLIP assay. We transfected Myc-tagged DDX17 into HEK293 cells and subsequently treated them with biotin-labelled Alu RNA (SEQ ID NO: 86) and subjected them to UV crosslinking (FIG. 8A). Immunoprecipitation of the cell lysate with anti-Streptavidin (FIG. 8B) or Myc antibody (FIG. 8C) pulled down biotin-labelled Alu RNA (SEQ ID NO: 86), as detected by northern blotting. Immunoblotting of myc and northern blotting of Alu RNA (SEQ ID NO: 86) showed that Alu RNA (SEQ ID NO: 86) interacted with DDX17 (FIG. 8C). Next, we tested the hypothesis that DDX17 interacts with NLRC4. In Alu RNA (SEQ ID NO: 86) treated mouse BMDMs, immunofluorescence studies revealed colocalization of DDX17 and NLRC4 (FIG. 9). A physical interaction between DDX17 and NLRC4 was identified in Alu RNA (SEQ ID NO: 86) treated HEK293 cells using a FLAG-NLRC4 expression system and immunoprecipitation (FIG. 10).

We next examined whether DDX5 or DDX17 was the sensor for Alu RNA (SEQ ID NO: 86) induced NLRC4 inflammasome activation. Due to the high degree of similarity in the sequences of DDX5 and DDX17, we designed 3 siRNAs targeting DDX5 alone, DDX17 alone, or both DDX5 and DDX17, and confirmed their target efficiency in THP1 cells (FIG. 11A). Both the DDX5/17 siRNA and the DDX17 siRNA reduce caspase-1 cleavage in Alu RNA (SEQ ID NO: 86) treated cells, whereas the DDX5 siRNA did not do so (FIG. 11A). Recent evidence suggests that DDX5 and DDX17 along with Drosha a core RNA-specific endoribonuclease is involved in miRNA micro processing. Therefore, we tested whether Drosha is also involved in SINE RNA-DDX17-NLRC4 axis. So, we transfected siDrosha or siDDX17 or siControl in mock or Alu RNA (SEQ ID NO: 86) treated THP1 cells and examined caspase-1 activation by immunoblotting. siDDX17 inhibited the levels of caspase-1 (FIG. 11B) whereas siDrosha did not. These observations implicate that DDX17 mediated SINE RNA induced NLRC4 inflammasome is independent of microprocessor function.

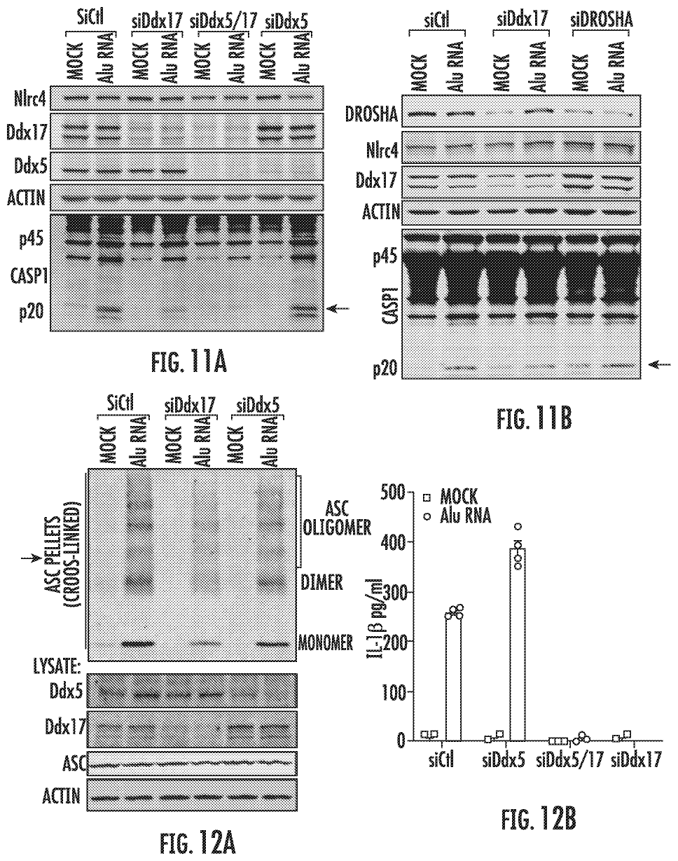

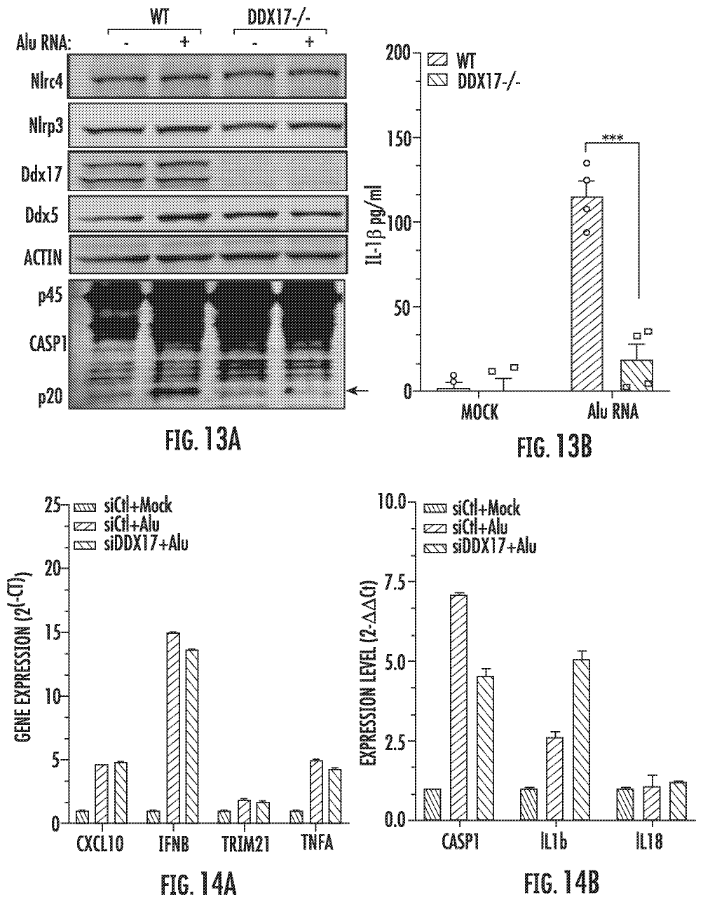

Then we examined whether siDDX17 could also inhibit ASC oligomerization and IL-1β release in THP1 cells. Immunoblotting revealed that only siDDX17 prevented the formation of ASC oligomers (FIG. 12A) compared to siControl and siDDX5. ELISA readout shows that IL-β was significantly reduced in siDDX17 and siDDX5/17 compared to siDDx5/control (FIG. 12B). All these data suggest that DDX17 is the sensor for SINE RNA-induced NLRC4 inflammasome activation. To further confirm that DDX17 is the sensor for NLRC4, we investigated caspase-1 cleavage by immunoblotting and IL-1β secretion by ELISA in Alu RNA (SEQ ID NO: 86) or mock treated THP1 cells and DDX17 KO BMDMs respectively. Caspase-1 was significantly reduced in DDX17 KO BMDMs compared to WT cells treated with Alu RNA (SEQ ID NO: 86); FIG. 13A). Furthermore, levels of IL-1β was significantly inhibited in DDX17 KO BMDMs as well as siDDX17, siDDX5/17 transfected THP1 cells further providing the basis for DDX17 as the sensor for NLRC4 (FIG. 13B).

We previously reported that Alu RNA (SEQ ID NO: 86) induces a type I interferon (IFN) response and inflammatory priming in BMDMs. Since DDX17 is essential for Alu RNA (SEQ ID NO: 86) induced inflammasome activation, we tested whether DDX17 knockdown could affect downstream events of the inflammasome cascade such as the type I IFN response. The expression of caspase-1, CXCL10, IFNβ, IL-18 and IL-1β, genes was upregulated in response to treatment with Alu RNA (SEQ ID NO: 86) with the exception of caspase-1. DDX17 siRNA did not affect the expression levels of these genes (FIGS. 14A and 14B).

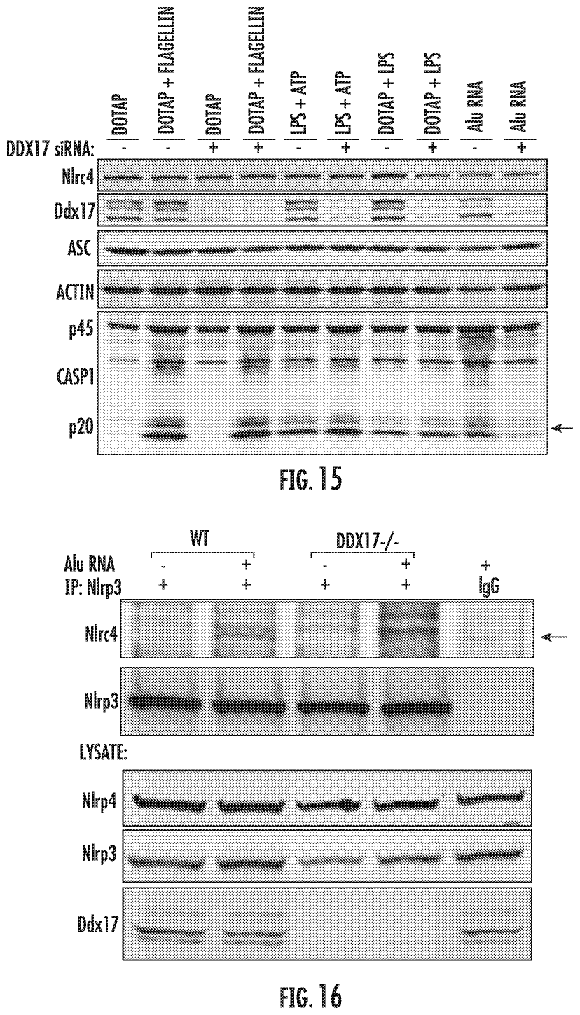

We finally examined whether DDX17 has a role in conventional NLRC4 activation by flagellin that requires NAIP and LPS+ATP dependent NLRP3 inflammasome activation. We treated mouse BMDMs with flagellin or LPS+ATP and transfected with siDDX17 and examined NLRC4 or NLRP3 dependent caspase-1 cleavage. Immunoblotting show that DDX17 knockdown does not affect flagellin and LPS+ATP induced NLRC4 and NLRP3 inflammasome respectively as indicated by unchanged caspase-1 levels (FIG. 15). These data support the idea that DDX17 is not involved in flagellin induced NLRC4 and LPS+ATP NLP3 inflammasome.

### Example 3

### NLRP3 but not NAIP is Required for Alu RNA-Induced DDX17-NLRC4 Activation

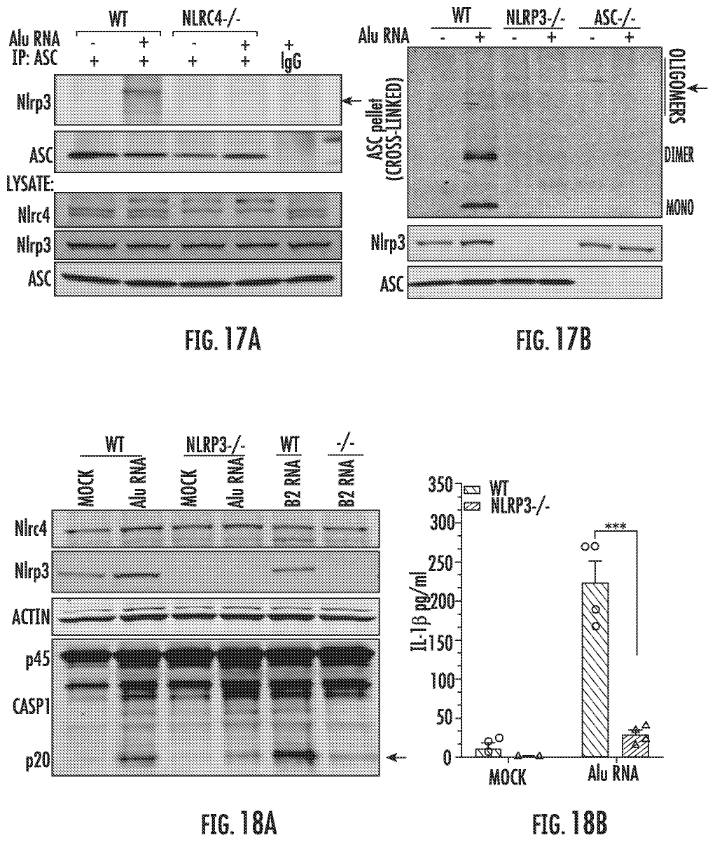

We next investigated whether Alu RNA (SEQ ID NO: 86) induced DDX17 could potentially interact with NLRs other than NLRC4. Immunoprecipitation tandem-Mass spectrometry identified that Alu RNA (SEQ ID NO: 86) induced DDX17 also interacts with NLRP3 peptides compared to mock treated DDX17 (see Tables 3 and 4). So, we tested whether interaction of Alu RNA (SEQ ID NO: 86) with DDX17 could recruit both NLRC4 and NLRP3 inflammasomes. We treated WT and DDX17 KO BMDMs with Alu RNA (SEQ ID NO: 86) and immunoprecipitated NLRP3 to examine for NLRC4 expression. Immunoblotting revealed that NLRC4 is only expressed in WT but not DDX17 KO BMDMs suggesting that Alu RNA (SEQ ID NO: 86) and DDX17 complex recruited NLRP3 and NLRC4 inflammasome (FIG. 16). We next asked if NLRC4 activity is functionally relevant for NLRP3-ASC interaction. We immunoprecipitated ASC in Alu RNA (SEQ ID NO: 86) treated WT and NLRC4 KO BMDMs and examined NLRP3 expression. Immunoblotting revealed that NLRP3 expression is completely inhibited in NLRC4 KO BMDMs suggesting that NLRP3-ASC interaction is inhibited in absence of NLRC4 (FIG. 17A). Furthermore, ASC monomers and dimers only appeared in WT BMDMs treated with Alu RNA (SEQ ID NO: 86) but not in NLRP3 or ASC KO BMDMs (FIG. 17B). We next assessed the functional role of NLRP3 in SINE RNA induced inflammasome. WT and NLRP3 KO BMDMs were subjected to mock or Alu RNA (SEQ ID NO: 86) treatment to examine NLRC4, caspase-1 and IL-1β levels. Immunoblotting results show that SINE RNA induced inflammasome is blocked in NLRP3 KO BMDMs without affecting the NLRC4 expression (FIG. 18A). Similarly, IL-1β levels were significantly inhibited in NLRP3 KO compared to WT BMDMs (FIG. 18B). These data suggest that NLRP3 is required but not essential for SINE RNA induced NLRC4 inflammasome.

TABLE 3
Immunoprecipitation Tandem-Mass Spec Identified
Interaction Between Ddx17 and NLRs

| | | | Ddx Complex (Alu RNA; (SEQ | | |
|---|---|---|---|---|---|
| Ddx Complex (Mock) | | | ID NO: 86) | | |
| | | Coverage | | | Coverage |
| Protein | Peptides | (%) | Protein | Peptides | (%) |
| Ddx17 | 22 | 31 | Ddx17 | 27 | 43 |
| Ddx5 | 10 | 17 | Ddx5 | 15 | 25 |
| Nlrc4 | 1 | 0.68 | Nlrc4 | 6 | 7.3 |
| Nlrp3 | 0 | 0 | Nlrp3 | 3 | 3.7 |
| Nlrp1 | 0 | 0 | Nlrp1 | 2 | 6.6 |
| Nlrp8 | 0 | 0 | Nlrp8 | 2 | 2.9 |

TABLE 4
Immunoprecipitation Tandem-Mass Spec
Identified Interaction Between Ddx17 and NLRs

| Protein | Sequence and SEQ ID NO:* | Prob (%) | Modifications* | Domain |
|---|---|---|---|---|
| NLRP3 | (R)YLEDLEDVDLKK(F) | 51 | | PYD |
| | (SEQ ID NO: 96) | | | |
| NLRP3 | (K)QQmESGKSLAQTSK(T) | 36 | Ox (+16) | NACHT |
| | (SEQ ID NO: 97) | | | |
| NLRP3 | (R)mNLFQKEVDcEK(F) | 82 | Ox (+16); | NACHT |
| | (SEQ ID NO: 98) | | Carb (+57) | |
| NLRC4 | (R)QFGALTAEVGDmTEDSAQALIR(E) | 41 | Ox (+16) | NACHT |
| | (SEQ ID NO: 99) | | | |
| NLRC4 | (R)YTcGSSVEATR(A) | 97 | Carb (+57) | NACHT |
| | (SEQ ID NO: 100) | | | |
| NLRC4 | (R)LPGGLTDSLGNLK(N) | 98 | | LRR3 |
| | (SEQ ID NO: 101) | | | |
| NLRC4 | (K)ILAQNLHNLVK(L) | 100 | | LRR6 |
| | (SEQ ID NO: 102) | | | |
| NLRC4 | (R)ILGAFFGK(N) | 35 | | LRR9 |
| | (SEQ ID NO: 103) | | | |
| NLRC4 | (K)EFLPDPALVR(K) | 97 | | LRR11 |
| | (SEQ ID NO: 104) | | | |

*Ox: oxidation; Carb: carbamidomethyl modification; Residues in parenthesis
indicate preceding amino acid residue; lowercase letters indicate site(s) of
mofifications.

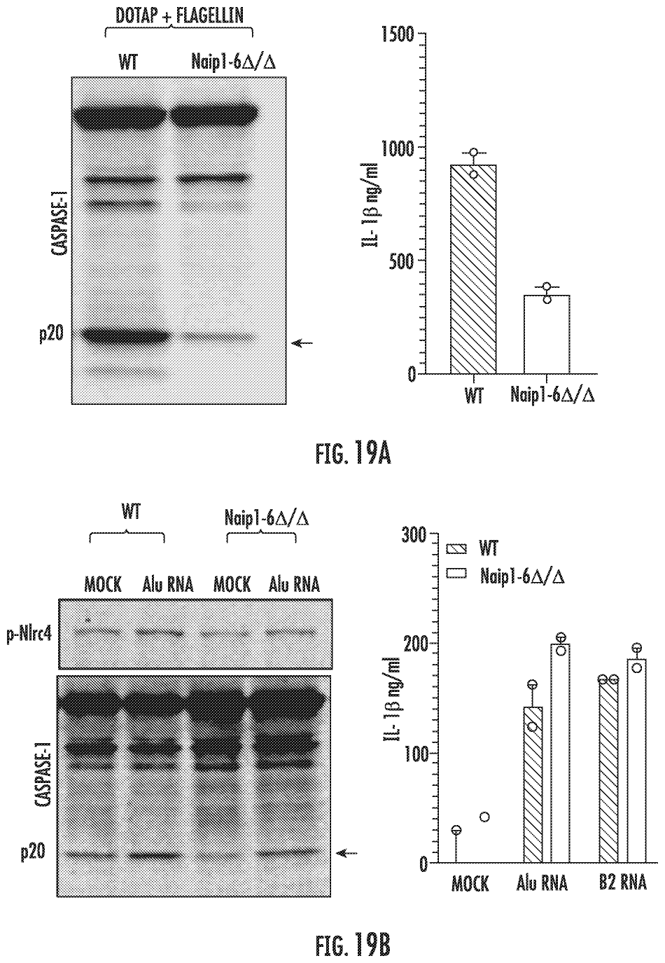

The NAIP family of proteins function to assist NLRC4 inflammasome activation by acting as dedicated pathogen sensors. Previous studies have shown that mouse NAIPs form complexes with NLRC4 and bacterial ligands to activate the inflammasome. For example, NAIP5 and NAIP2 form complexes with NLRC4 and flagellin or T35SS components of Salmonella species respectively. Later studies revealed that, similar to NAIP5, NAIP6 can also recognize flagellin, while NAIP1 activates NLRC4 in response to the T3SS protein PrgI. In humans, however, only one copy of NAIP exists through which NLRC4 is activated. Thus, NAIPs are essential for NLRC4 inflammasome activation in mice and humans. Hence, we sought to determine whether Alu RNA (SEQ ID NO: 86) induced NLRC4 activation is NAIP-dependent. First, we examined NLRC4-mediated caspase-1 activation by flagellin, which requires NAIPs, using NAIP1-6 KO mouse cells. Consistent with previous studies, BMDMs isolated from NAIP1-6 KO mice failed to activate the NLRC4 inflammasome when stimulated with flagellin, as evidenced by significant inhibition of cleaved caspase-1 product (p20 subunit; FIG. 19A). Similar to findings reported in the literature, IL-1β levels estimated by ELISA were also reduced significantly in NAIP1-6 KO cells (FIG. 19A).

Then we examined whether there is a difference in Alu RNA (SEQ ID NO: 86) induced NLRC4 phosphorylation in BMDMs harvested from WT and NAIP 1-6 KO mice. Immunoblotting revealed that NLRC4 phosphorylation is not inhibited in NAIP 1-6 KO mice indicating that NAIP may not be involved in Alu RNA (SEQ ID NO: 86) induced NLRC4 activation (FIG. 19B). Next, we treated C57BL/6J WT, NLRC4 KO and NAIP1-6 KO BMDMs with Alu RNA (SEQ ID NO: 86) and monitored caspase-1 activation by western blotting. Interestingly, Alu RNA (SEQ ID NO: 86) induced caspase-1 cleavage is not blocked in NAIP1-6 KO BMDMs (FIG. 19B) suggesting that Alu RNA-induced NLRC4 inflammasome activation is independent of NAIPs. To further confirm Alu RNA-induced NLRC4 activation is NAIP-independent we treated WT and NAIP 1-6 KO BMDMs with SINE RNA and examined IL-1β secretion using ELISA. IL-1β secretion was not inhibited in response to SINE RNA treatment (FIG. 19B). Collectively our results introduce Alu RNA as a trigger of a novel NAIP-independent, non-canonical NLRC4 inflammasome pathway.

We have previously shown that DICER deficiency mediated accumulation of Alu RNA induces NLRP3 inflammasome activation. So we examined whether DICER knockdown could also induce DDX17-NLRC4-NLRP3 signaling. WT and DDX17 KO BMDMs were transfected with siDICER to examine NLRC4 phosphorylation and caspase-1. Immunoblotting data show that dicer deficiency induced the phosphorylation of NLRC4 and caspase-1 activation in WT which is blocked in DDX17 KO BMDMs (FIG. 20A). We then determined phosphorylation of NLRC4 and caspase-1 activation in WT, NLRC4 KO and NLRP3 KO BMDMs after transfection with siDICER or sicontrol. Phosphorylation of NLRC4 is only blocked in NLRC4 KO but not in WT or NLPR3 KO BMDMs (FIG. 20B). Furthermore, siDICER induced the activation of caspase-1 in WT BMDMs which is blocked in NLRC4 and NLRP3 KO BMDMs (FIG. 20B). All these data support the notion that DDX17-NLRC4-NLRP3 signaling is required for DICER knockdown induced inflammasome activation.

### Example 4

### DDX17-Mediated Non-Canonical NLRC4-NLRP3 Inflammasome as a Therapeutic Target for Age Related Macular Degeneration

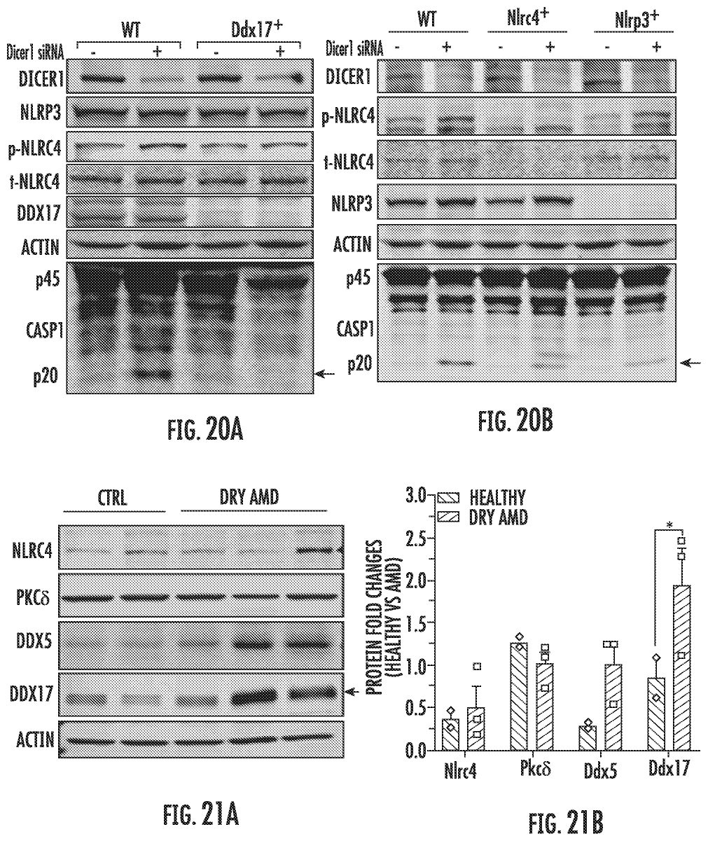

Next, we tested whether human eyes with GA express DDX17 and NLRC4. Immunoblotting revealed an DDX17 and NLRC4 abundance in the RPE/choroid of GA eyes compared to control eyes (FIGS. 21A and 21B). Since DDX17 interacted with NLRC4 in vitro we investigated if this is also true for patients with AMD. Using PLA assay on human AMD tissue we discovered that NLRC4 interacted with DDX17 compared to healthy controls (FIG. 22). Collectively, these data provide evidence of NAIP and NLRC4 involvement in human GA, mirroring the functional data in BMDM and THP1 cell culture studies.

Previously we established that accumulation of Alu RNA (SEQ ID NO: 86) due to DICER1 loss activates the NLRP3 inflammasome in mice and humans, causing RPE degeneration in a caspase-1-dependent manner. Since Alu RNA (SEQ ID NO: 86) activates NLRC4 in mouse BMDMs, we next performed in vivo experiments to examine whether NLRC4 inflammasome activation can induce RPE degeneration in mice. We tested whether enforced expression of constitutively active NLRC4 can induce RPE degeneration in mice. Subretinal injection of pNLRC4^{T3375}-IRES-GFP, which is constitutively active, but not pNLRC4^{WT}-IRES-GFP, induced RPE degeneration in WT mice (FIGS. 23A and 23B).

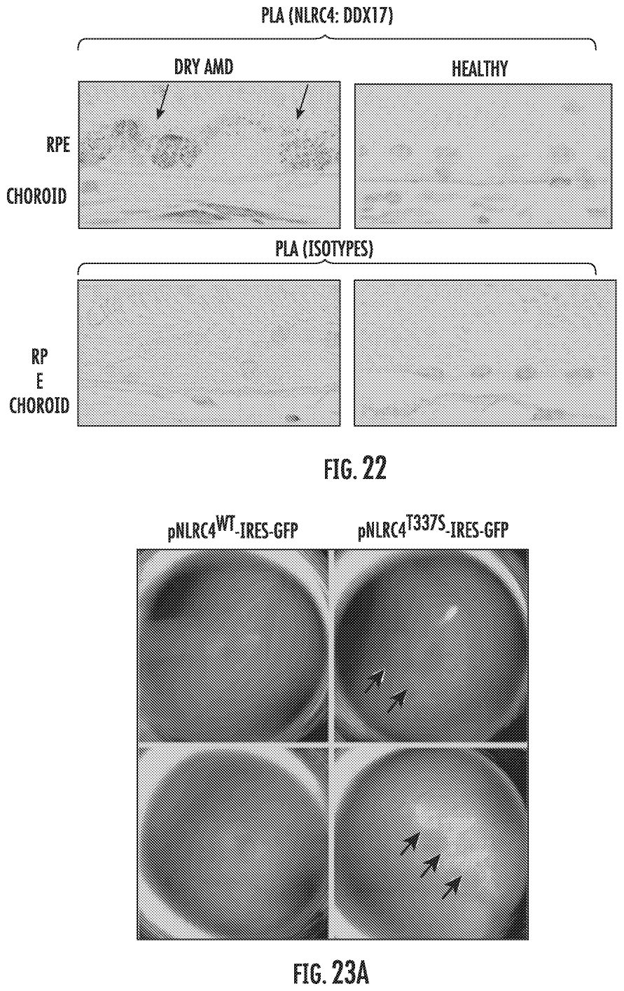

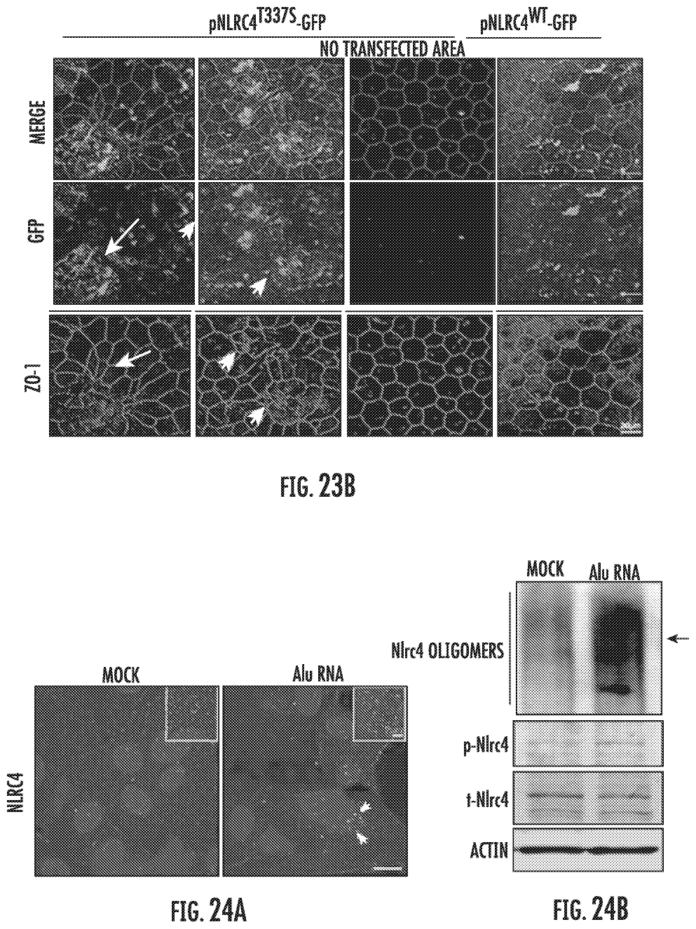

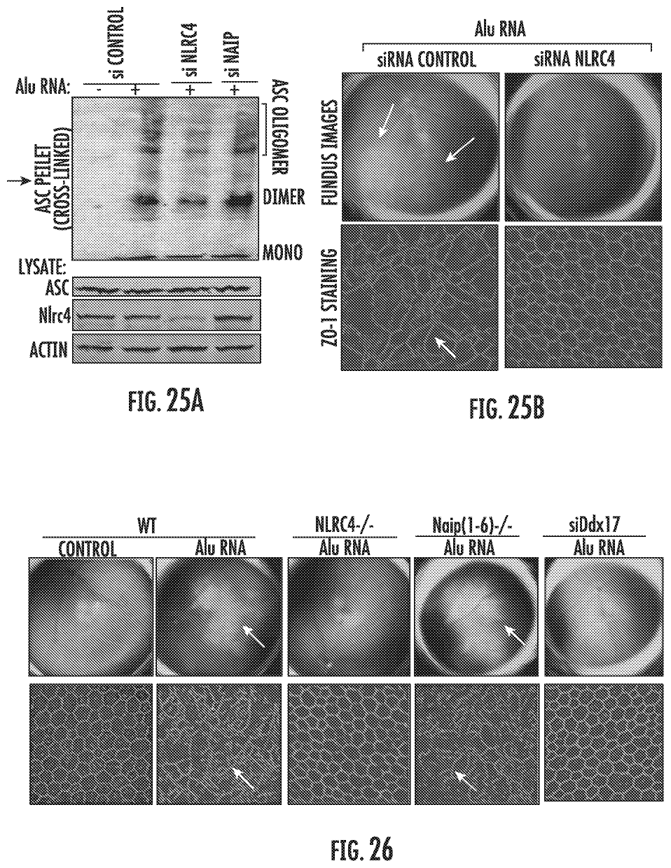

Next, we asked whether Alu RNA (SEQ ID NO: 86) also triggered NLRC4 inflammasome activation in human RPE cells. We performed immunofluorescence staining experiments to examine whether Alu RNA (SEQ ID NO: 86) can induce NLRC4 speck formation in human RPE cells. We noticed punctate structures resembling specks of NLRC4 in human RPE cells in response to Alu RNA (SEQ ID NO: 86) treatment (FIG. 24A). Furthermore, human RPE cells transfected with Alu RNA (SEQ ID NO: 86) induced the formation of NLRC4 oligomers compared to mock transfected cells (FIG. 24B). Collectively, these data demonstrate the existence of an NLRC4 signaling pathway in RPE cells which is activated by Alu RNA (SEQ ID NO: 86). We further investigated downstream events of NLRC4 inflammasome activation by studying NLRC4-induced ASC oligomerization, a critical step involved in caspase-1 cleavage. NLRC4 siRNA, when co-administered with Alu RNA (SEQ ID NO: 86), blocked ASC oligomerization in vitro (FIG. 25A) and RPE degeneration in vivo (FIG. 25B).

Finally, we sought to investigate whether interfering with NLRC4 inflammasome blocks SINE RNA-induced RPE degeneration. We delivered Alu RNA (SEQ ID NO: 86) or Alu RNA (SEQ ID NO: 86)+siDDX17 subretinally in WT or NLRC4 KO or NAIP1-6 KO mice and examined for RPE degeneration using fundus photos and ZO-1 staining as described previously. Alu RNA (SEQ ID NO: 86) induced RPE degeneration only in WT and NAIP1-6 KO mice whereas NLRC4 KO and siDDX17 injected mice were rescued from this phenotype suggests that interfering with NLRC4 inflammasome signaling blocks SINE RNA-induced RPE degeneration (FIG. 26).

### Example 5

### NRTIs Block NLRC4 Inflammasome-Induced RPE Degeneration

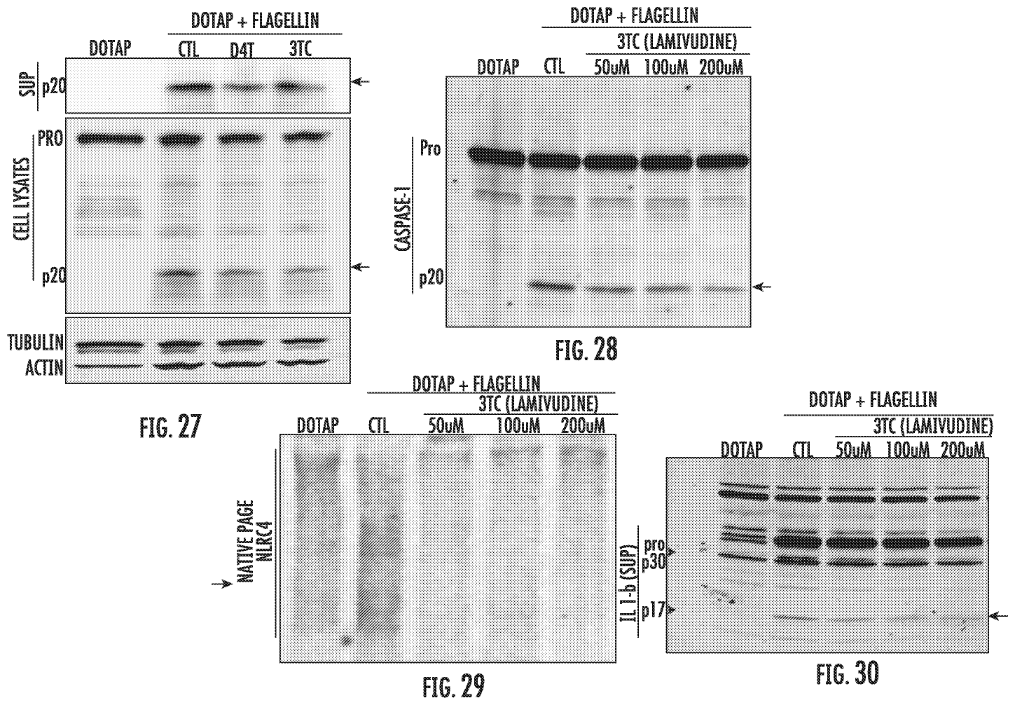

Nucleoside reverse transcriptase inhibitors (NRTIs) are HIV therapeutics that inhibit retrovirus replication. We have previously shown that NRTIs inhibit P2X7-mediated NLRP3 inflammasome activation by an endogenous retroelement Alu RNA (SEQ ID NO: 86). Activation of NLRP3 inflammasome by Alu RNA (SEQ ID NO: 86) causes the death of retinal pigment epithelium (RPE) in geographic atrophy, a severe form of AMD. We previously reported that intraperitoneal administration of NRTIs (D4T and AZT) prevented Alu RNA (SEQ ID NO: 86) induced RPE degeneration in mice. Currently, multiple NLRP3 inflammasome inhibitors such as MCC950, CY09 are available however, there are no inhibitors of NLRC4. So, a need exists to develop inhibitors of NLRC4 inflammasome to study NLRC4 driven diseases. Here, we show for the first time that NRTIs block flagellin induced NLRC4 inflammasome. We pretreated flagellin transfected BMDMs with DT4 and 3TC to examine caspase-1 activation. Immunoblotting studies show that D4T and 3TC blocked flagellin induced caspase-1 activation compared to controls (FIG. 27). Moreover, we have seen a dose-dependent inhibition of caspase-1 activation when treated with 3TC (FIG. 28). Together all these data suggest that NRTIs block NLRC4 induced caspase-1 activation.

NLRC4 oligomerization is another hallmark of inflammasome activation where high-molecular mass NLRC4 protein complexes are assembled. So, we next examined whether 3TC can also block flagellin induced NLRC4 oligomerization. 3TC pretreated BMDMs were transfected with flagellin and cell lysates were resolved using native polyacrylamide gel electrophoresis (NATIVE-PAGE) to detect NLRC4 oligomerization. Immunoblotting revealed that flagellin induced NLRC4 oligomers in control which was dose-dependently inhibited by 3TC suggesting that NRTIs block flagellin induced NLRC4 inflammasome (FIG. 29). Furthermore, activated NLRC4 inflammasome induce the production of IL-1β cytokine, so we examined whether 3TC can also block flagellin induced NLRC4 dependent IL-1β production. Immunoblotting data show that IL-1β production is inhibited by 3TC in a dose dependent manner in flagellin transfected BMDMs (FIG. 30). Next, we modified AZT and 3TC to generate 2-ethyl AZT (K8) and 3-methyl 3TC (K9) and tested their ability to block flagellin induced NLRC4 dependent caspase-1 activation. Immunoblotting results show that K8 and K9 blocked caspase-1 activation induced by flagellin compared to control (FIG. 31). Collectively, all these data suggest that NRTIs and modified NRTIs inhibit the activation of NLRC4 inflammasome.

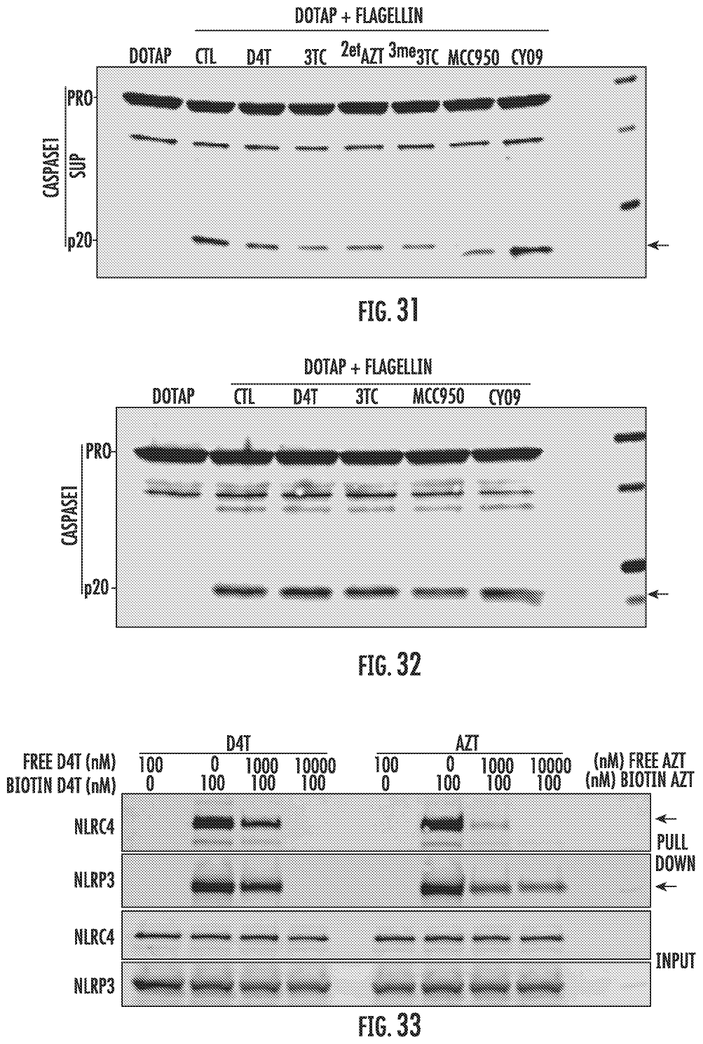

Finally, we sought to investigate the mechanism through which NRTIs inhibit NLRC4 inflammasome and asked whether this is NLRP3 dependent. We transfected WT and NLRP3 KO BMDMs with flagellin to examine caspase-1 activation using immunoblot. Flagellin induced caspase-1 activation is blocked in NLRP3 deficient BMDMs suggesting that NLRC4 activation is NLRP3 dependent (FIG. 32). To further confirm the mechanism of action of NRTIs, we investigated whether they directly bind to NLRP3/NLRC4 complex in a reconstituted system. We treated flagellin transfected BMDMs with free and biotin labeled D4T and AZT and pulled down biotin. Immunoblotting revealed that D4T and AZT interacted with NLRP3 and NLRC4 further confirming their mechanism of action (FIG. 33). In summary, all these data suggest that NRTIs inhibit flagellin induced NLRC4 activation by directly binding to NLRP3/NLRC4 complex.

### Example 6

### PKCδ Inhibition Blocks NLRC4 Phosphorylation and Alu RNA-Induced Caspase-1 Activation

Wild-type BMDMs were pre-treated with indicated dose of the PKCδ inhibitor Rottlerin (Signa-Aldrich Corp., St. Louis, Mo., United States of America) and the LRRK2 inhibitor GSK2578215A (Sigma-Aldrich) for 1 hour, and then stimulated with Alu RNA (SEQ ID NO: 86) transfection (100 pmol). Supernatant and cell lysates were collected for Caspase-1 cleavage and p-NLRC4 blots. Results indicated that PKCδ inhibitor inhibited NLRC4 phosphorylation and Caspase-1 activation induced by Alu RNA (SEQ ID NO: 86).

### Example 7

### PKCδ and NLRC4 Phosphorylation (S533) are Required for Alu RNA-Induced Inflammasome Activation

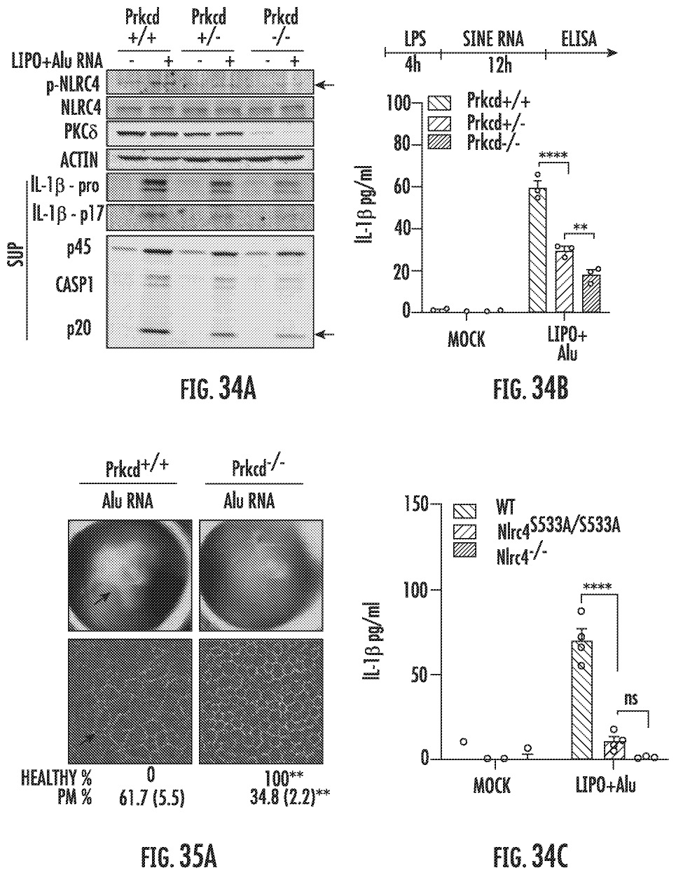

Wild-type, Prkcd^{-/+}, and Prkcd^{-/-} BMDMs were transfected with Alu RNA (SEQ ID NO: 86 at 100 pmol) for 12 hours. Supernatants were collected for measuring Caspase-1, IL-1β cleavage, and IL-1β release. Cell lysates were collected for p-Nlrc4, Nlrc4, PKCδ, and actin blots. The results presented in FIGS. 34A and 34B showed that Caspase-1, IL-1β cleavage, and IL-1β release were impaired in Prkcd^{-/-} BMDMs.

Wild-type and Nlrc4^{S533A/S533A} BMDMs were transfected with Alu RNA (SEQ ID NO: 86 at 100 pmol) for 12 hours. Supernatants were collected for measuring IL-1β release by ELISA. The results shown in FIG. 34C indicated that IL-1β release was impaired in Nlrc4^{S533A/S533A} BMDMs.

### Example 8

### PKCδ-Mediated NLRC4 Phosphorylation is Required for Alu RNA-Induced RPE Degeneration

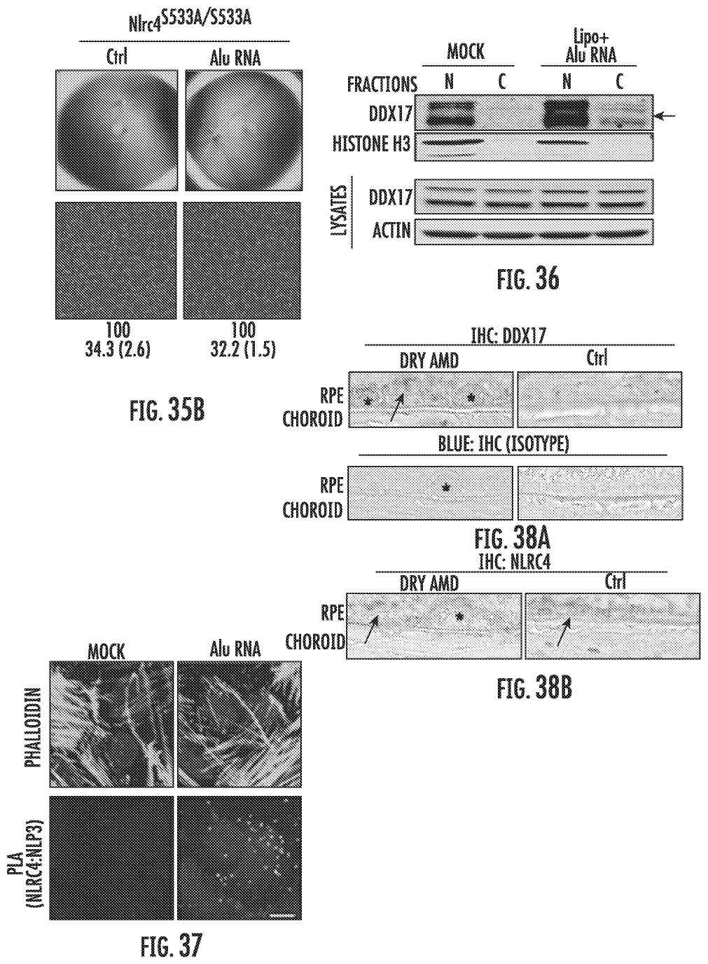

Wild-type, Prkcd^{-/-}, and Nlrc4^{S533A/S533A} mice were subretinally injected with Alu RNA (SEQ ID NO: 86). Fundus images and ZO-1 fluorescent images are presented in FIGS. 35A and 35B. As shown, Alu RNA (SEQ ID NO: 86) induced RPE degeneration was blocked in Prkcd^{-/-} and Nlrc4^{S533A/S533A} mice.

### Example 9

### Alu RNA Induces DDX17 Translocation in Human Cells

Human monocytes (THP-1) were treated with Alu RNA (SEQ ID NO: 86 at 100 pmol). Cell lysates were collected and subjected to cell fractionation. Immunoblots of DDX17 and Histone H3 were used to evaluate the subcellular distribution of DDX17. As shown in FIG. 36, Alu RNA (SEQ ID NO: 86) treatment induced DDX17 translocation from the nucleus to the cytoplasm, and DDX17 co-localized with cytosolic Alu RNA (SEQ ID NO: 86).

### Example 10

### Alu RNA Induces the Assembly of NLRC4 and NLRP3 Complex

Human RPE cells were treated with biotinylated Alu RNA (SEQ ID NO: 86 at 100 pmol). The assembly of NLRC4 and NLRP3 complex was evaluated by Proximity Ligation Assay (PLA). The results are presented in FIG. 37, which showed that Alu RNA (SEQ ID NO: 86) transfection induced the assembly of NLRC4 and NLRP3 complex in human RPE cells.

### Example 11

### The Expression of DDX17 is Increased in the RPE of Human Donor Eyes with Dry AMD

The expression of DDX17 and NLRC4 proteins was measured in human donor eyes with dry AMD via immunohistochemistry, and the results are presented in FIGS. 38A and 38B. As shown therein, the expression of DDX17 was increased in the RPE of human donor eyes with dry AMD.

### Discussion of the Examples

NLR family CARD domain containing (NLRC) 4 is a cytosolic protein expressed by epithelial and innate immune cells. The NLRC4 protein assembles an inflammasome complex with apoptosis speck-like protein and caspase-1 to promote the maturation of pro-inflammatory cytokines interleukin (IL)-1β, IL-18, and gasdermin D (Gsdmd), thereby inducing an inflammatory form of cell death known as pyroptosis. The NLRC4 inflammasome is best known for regulating antibacterial immunity by indirectly sensing bacterial flagellin and type III secretory system (T3SS) with the help of pathogen-sensing proteins known as NLR family apoptosis inhibitory proteins (NAIPs). Here we show that short interspersed nuclear elements (SINE) transcripts, non-bacterial molecules, also can induce NLRC4 inflammasome activation in mouse and human macrophages and in retinal pigment epithelium (RPE) cells. In contrast to flagellin-induced NLRC4 activation, which is dependent on NAIPs, we show that mice deficient in all NAIP genes remain susceptible to SINE RNA-induced NLRC4 activation. Through an unbiased manner, we identified DDX17, a member of the DEAD box family of RNA helicases, as a sensor for SINE RNA-induced NLRC4 inflammasome activation. We mechanistically found that upon binding with SINE RNA, Ddx17 induced dual recruitment of NLRC4 and NLRP3, as well as ASC molecules result in Caspase-1 activation and IL-1β release. Therapeutic manipulation of Ddx17-Nlrc4-NLRP3 signaling protected against SINE RNA-induced RPE degeneration in an animal model of age-related macular degeneration (AMD). Finally, we show that nucleoside reverse transcriptase inhibitors (NRTIs), which are currently used as HIV therapeutics, possess inhibitory activity against NLRC4 inflammasome. Collectively, these data highlight the discovery of a sterile NAIP-independent non-canonical NLRC4 inflammasome pathway that has implications in AMD, the most common cause of irreversible central blindness, and that NRTIs function to inhibit this NLRC4 inflammasome pathway.

Our data identified a novel NAIP independent non-canonical NLRC4 inflammasome pathway activated by SINE RNA in both in vivo and in human cell culture studies. Furthermore, we have identified DDX17 as the novel sensor for SINE RNA mediated NLRC4 inflammasome. Interestingly, we found that NLRC4 inflammasome components are dysregulated in human AMD eyes. Interfering with NLRC4 inflammasome pathway signaling reversed the RPE degeneration induced by SINE RNAs. Furthermore, we show that NRTIs and modified NRTIs effectively block NLRC4 induced caspase-1 activation. These data implicate NLRC4 inflammasome as a key player in the pathogenesis of AMD.

To date, three models best explain the activation of the NLRC4 inflammasome. Firstly, pathogen-associated molecular patterns (PAMPs), including bacterial products such as flagellin and T3SS proteins, activate the NLRC4 inflammasome through NAIPs. This mechanism is vital for defense against enteric pathogens. Secondly, inherited mutations in NLRC4 results in severe auto inflammatory disease in infants. Finally, several studies have reported that endogenous stimuli, including brain injury, age related nucleotide metabolism, or lysophosphatidylcholine, can also induce NLRC4-dependent inflammasome activation.

The molecular mechanisms governing this sterile NLRC4 inflammasome activation is largely unknown. Using AMD as a model, our findings for the first time revealed a NAIP independent, non-canonical NLRC4 inflammasome induced by endogenous SINE RNA species. Upon SINE RNA recognition by Ddx17, NLRC4 is phosphorylated by PKCδ to promote interaction between Ddx17 and NLRC4, resulting in oligomerization of NLRC4, ASC speck formation, caspase-1 activation, and IL-18 release. These findings have implications for the pathology of AMD.

Using immunoblotting studies, we discovered that NLRC4 inflammasome activation was NLRP3 dependent and that NRTIs could directly bind to NLRP3-NLRC4 complex to inhibit its activation. These findings support the ability of NRTIs and modified NRTIs to treat and/or prevent NLRC4 inflammasome driven diseases and to act as NLRC4 inhibitors.

### REFERENCES

All references listed below, as well as all references cited in the instant disclosure, including but not limited to all patents, patent applications and publications thereof, scientific journal articles, and database entries (e.g., GENBANK® biosequence database entries and all annotations available therein) are incorporated herein by reference in their entireties to the extent that they supplement, explain, provide a background for, or teach methodology, techniques, and/or compositions employed herein.
Altschul et al. (1990a) Basic local alignment search tool. J Mol Biol 215:403-410.
Altschul et al. (1990b) Protein database searches for multiple alignments. Proc Natl Acad Sci USA 87:14:5509-5513.
Altschul et al. (1997) Gapped BLAST and PSI-BLAST: a new generation of protein database search programs. Nucleic Acids Res 25:3389-3402.
Bass (2001) The short answer. Nature 411:428-429.
Elbashir et al. (2001) Duplexes of 21-nucleotide RNAs mediate RNA interference in cultured mammalian cells. Nature 411:494-498.
GENBANK® Accession Nos. NM_001033367.3; NM_001040187.1; NM_006386.5; NM_021209.4; NP_001028539.1; NP_001035277.1; NP_006377.2; NP_067032.3.
Gross & Mienhofer (eds.) (1981) The Peptides, Volume 3, Academic Press, New York, N.Y., United States of America.
Harlow & Lane (1988) Antibodies: A Laboratory Manual, Cold Spring Harbor Laboratory Press, Cold Spring Harbor, N.Y., United States of America.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Murphy et al. (2010) Antiviral activity and tolerability of amdoxovir with zidovudine in a randomized double-blind placebo-controlled study in HIV-1-infected individuals. Antivir Ther 15(2):185-192.
Otto (2004) New nucleoside reverse transcriptase inhibitors for the treatment of HIV infections. Curr Opin Pharmacol 4(5):431-436.
PCT International Publication Nos. WO 99/07409; WO 99/32619; WO 2000/001846; WO 2000/044895; WO 2000/044914; WO 2001/036646; WO 2001/029058; WO 2008/007382; WO 2014/176532; WO 2014/183147; WO 2019/074884.
Shelton et al. (1993) Zalcitabine. Ann Pharmacother 27(4):480-489.
U.S. Patent Application Publication Nos. 2011/0150997; 2019/0022115; 2019/0055273; 2019/0177326; 2019/0185508.
U.S. Pat. Nos. 5,663,159; 5,905,082; 6,294,540; 6,350,736; 6,417,191; 6,627,224; 7,589,078; 8,026,356; 8,183,370; 8,193,165; 9,126,971; 9,296,769.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating and/or preventing a disease, disorder, or condition associated with an NLR family CARD domain containing 4 (NLRC4) inflammasome biological activity, the method comprising administering to a subject in need thereof a composition comprising, consisting essentially of, or consisting of a nucleoside reverse transcriptase inhibitor (NRTI), wherein the administering is via an route and in an amount effective for reducing the NLRC4 inflammasome biological activity, thereby treating and/or preventing the disease, disorder, or condition associated with the NLRC4 inflammasome biological activity.

2. The method of claim 1, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

3. The method of claim 1, wherein the disease, disorder, or condition associated with the NLRC4 inflammasome biological activity is a disease of the retinal pigmented epithelium (RPE), optionally age-related macular degeneration (AMD) and/or geographic atrophy.

4. The method of claim 1, further comprising administering to the subject in need thereof at least one additional inhibitor of the NLRC4 inflammasome biological activity.

5. The method of claim 4, wherein the at least one additional inhibitor the NLRC4 inflammasome biological activity comprises, consists essentially of, or consists of an inhibitor of a biological activity of at least one molecule or complex selected from the group consisting of NLRC4, NLRP3, caspase-1 (CAS-1), cyclic GMP-AMP synthase (CGAS), caspase-4 (CAS-4), stimulator of interferon genes-1 (STING1), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR).

6. The method of claim 5, wherein the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

7. The method of claim 5, wherein the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

8. A method for inhibiting NLRC4-induced caspase-1 activation in a cell, the method comprising contacting an NLRC4 gene product and/or a complex of an NLRC4 gene product and an NLR family pyrin domain containing 3 (NLRP3) gene product with an effective amount of a composition comprising, consisting essentially of, or consisting of a nucleoside reverse transcriptase inhibitor (NRTI), whereby NLRC4-induced caspase-1 activation is inhibited in the cell.

9. The method of claim 8, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

10. The method of claim 8, wherein the cell is present in a subject, optionally a mammalian subject, further optionally a human subject.

11. The method of claim 8, further comprising administering to the subject in need thereof at least one additional inhibitor of the NLRC4 inflammasome biological activity.

12. The method of claim 11, wherein the at least one additional inhibitor the NLRC4 inflammasome biological activity comprises, consists essentially of, or consists of an inhibitor of a biological activity of at least one molecule or complex selected from the group consisting of NLRC4, NLRP3, caspase-1 (CAS-1), cyclic GMP-AMP synthase (CGAS), caspase-4 (CAS-4), stimulator of interferon genes-1 (STING1), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR).

13. The method of claim 11, wherein the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

14. The method of claim 11, wherein the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

15. A method for inhibiting NLRC4-induced IL-1β release from a cell, the method comprising contacting an NLRC4 gene product and/or a complex of an NLRC4 gene product and an NLR family pyrin domain containing 3 (NLRP3) gene product with an effective amount of a composition comprising, consisting essentially of, or consisting of a nucleoside reverse transcriptase inhibitor (NRTI), whereby NLRC4-induced IL-1β release from the cell is inhibited.

16. The method of claim 15, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

17. The method of claim 15, wherein the cell is present in a subject, optionally a mammalian subject, further optionally a human subject.

18. The method of claim 8, wherein the NLRC4-induced caspase-1 activation and/or the NLRC4-induced IL-1β release is associated with a disease, disorder, or condition associated with an NLR family CARD domain containing 4 (NLRC4) inflammasome biological activity.

19. The method of claim 18, wherein the disease, disorder, or condition associated with the NLRC4 inflammasome biological activity is a disease of the retinal pigmented epithelium (RPE), optionally age-related macular degeneration (AMD) and/or geographic atrophy.

20. The method of claim 18, further comprising administering to the subject in need thereof at least one additional inhibitor of the NLRC4 inflammasome biological activity.

21. The method of claim 20, wherein the at least one additional inhibitor is selected from the group consisting of an antisense oligonucleotide, a small interfering RNA (siRNA), a short hairpin RNA (shRNA), an antibody or antigen-binding fragment thereof.

22. The method of claim 21, wherein the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

23. A method for inhibiting Alu-induced retinal pigmented cell (RPE) degeneration in a subject, the method comprising contacting an NLRC4 gene product and/or a complex of an NLRC4 gene product and an NLR family pyrin domain containing 3 (NLRP3) gene product in a cell of the subject with an effective amount of a composition comprising, consisting essentially of, or consisting of a nucleoside reverse transcriptase inhibitor (NRTI), whereby NLRC4-induced IL-1β release from the cell is inhibited.

24. The method of claim 23, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

25. The method of claim 23, wherein the cell is an RPE cell that present in a subject, optionally a mammalian subject, further optionally a human subject.

26. The method of claim 23, further comprising administering to the subject at least one additional treatment designed to protect the RPE from degradation.

27. The method of claim 26, wherein the at least one additional treatment comprises administering to the subject an inhibitor of a biological activity of at least one molecule or complex selected from the group consisting of NLRC4, NLRP3, caspase-1, cyclic GMP-AMP synthase (cGAS), caspase-4, stimulator of interferon genes (STING), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR).

28. The method of claim 27, wherein the inhibitor is a small interfering RNA (siRNA) or short hairpin RNA (shRNA) that targets a transcription product of a gene selected from the group consisting of NLRC4, NLRP3, caspase-1, cGAS, caspase-4, STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

29. The method of claim 27, wherein the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

30. A composition for use in treating and/or preventing a disease, disorder, or condition associated with an NLR family CARD domain containing 4 (NLRC4) inflammasome biological activity, the composition comprising, consisting essentially of, or consisting of a nucleoside reverse transcriptase inhibitor (NRTI).

31. A composition for use in inhibiting NLRC4-induced IL-1β release from a cell, the composition comprising, consisting essentially of, or consisting of a nucleoside reverse transcriptase inhibitor (NRTI).

32. A composition for use in inhibiting Alu-induced retinal pigmented cell (RPE) degeneration in a subject, the composition comprising, consisting essentially of, or consisting of a nucleoside reverse transcriptase inhibitor (NRTI).

33. The composition for use of, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

34. The composition for use of claim 30, wherein the composition is formulated for ocular delivery.

35. The composition for use of claim 30, wherein the composition further comprises an inhibitor of a biological activity of at least one molecule or complex selected from the group consisting of NLRC4, NLRP3, caspase-1, cyclic GMP-AMP synthase (cGAS), caspase-4, stimulator of interferon genes (STING), peptidyl-prolyl cis-trans isomerase F (PPIF), mitochondrial permeability transition pore (MPTP), Gasdermin D (GSDMD), interferon-beta (IFN-β), and interferon-α/β receptor (IFNAR).

36. The composition for use of claim 35, wherein the inhibitor is a small interfering RNA (siRNA) or short hairpin RNA (shRNA) that targets a transcription product of a gene selected from the group consisting of NLRC4, NLRP3, caspase-1, cGAS, caspase-4, STING, PPIF, MPTP, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.

37. The composition for use of claim 35, wherein the inhibitor is an antibody or antigen-binding fragment thereof that binds to a translation product of a gene selected from the group consisting of NLRC4, NLRP3, caspase-1, cGAS, caspase-4, STING, PPIF, MPTP, GSDMD, IFN-β, and IFNAR.

38. The method of claim 15, wherein the NLRC4-induced caspase-1 activation and/or the NLRC4-induced IL-1β release is associated with a disease, disorder, or condition associated with an NLR family CARD domain containing 4 (NLRC4) inflammasome biological activity.

39. The method of claim 38, wherein the disease, disorder, or condition associated with the NLRC4 inflammasome biological activity is a disease of the retinal pigmented epithelium (RPE), optionally age-related macular degeneration (AMD) and/or geographic atrophy.

40. The method of claim 18, further comprising administering to the subject in need thereof at least one additional inhibitor of the NLRC4 inflammasome biological activity.

41. The method of claim 40, wherein the at least one additional inhibitor is selected from the group consisting of an antisense oligonucleotide, a small interfering RNA (siRNA), a short hairpin RNA (shRNA), an antibody or antigen-binding fragment thereof.

42. The method of claim 41, wherein the inhibitor is a small interfering RNA (siRNA) or short hairpin RNA (shRNA) that targets a transcription product of a gene selected from the group consisting of NLRC4, NLRP3, CAS-1, CGAS, CAS-4, STING, PPIF, GSDMD, IFN-β, and IFNAR, optionally wherein the transcription product comprises, consists essentially of, or consists of a nucleotide sequence amino acids set forth in any of SEQ ID NOs: 1, 7, 21, 28, 35, 37, 39, 41, 43, 52, 54, 59, 61, 64, 66, 69, 71, 74, 76, 79, 81, and 84, further optionally wherein the siRNA or the shRNA comprises, consists essentially of, or consists of a nucleotide sequence as set forth in any of SEQ ID NOs: 3-6 and targets a human NLRC4 transcription product, SEQ ID NOs: 9-20 and targets a mouse Nlrc4 transcription product, SEQ ID NOs: 23-27 and targets a human DDX17 transcription product, SEQ ID NOs: 30-34 and targets a mouse Ddx17 transcription product, SEQ ID NO: 45 and targets a human CAS-4 transcription product, SEQ ID NOs: 46-51 and targets a human CAS-4 transcription product, SEQ ID NOs: 56-58 and targets a human CGAS transcription product, SEQ ID NO: 63 and targets a human STING1 transcription product, SEQ ID NO: 68 and targets a human PPIF transcription product, SEQ ID NO: 73 and targets a human GSDMD transcription product, SEQ ID NO: 78 and targets a human IFN-β transcription product, and SEQ ID NO: 83 and targets a human IFNAR transcription product.


SEQUENCE LISTING (104 sequences)

SEQ ID NO: 1 - DNA, 3385 bp, Homo sapiens
  CDS (276)..(3350)
aagcccctggctgtttatactccggagggtgtccccgtgcgtcatcggtggagtggaccaaaactggtgatctgtttgccctgtgtgaccttgcccagaaccctgctgactgagagaacacatctgctggaagtcctctgggattcaaggtacagggaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaaataaagtgtactcgaagccagtaaaaaaaaaaaaa

SEQ ID NO: 2 - protein, 1024 aa, Homo sapiens
MNFIKDNSRALIQRMGMTVIKQITDDLFVWNVLNREEVNIICCEKVEQDAARGIIHMILKKGSESCNLFLKSLKEWNYPLFQDLNGQSLFHQTSEGDLDDLAQDLKDLYHTPSFLNFYPLGEDIDIIFNLKSTFTEPVLWRKDQHHHRVEQLTLNGLLQALQSPCIIEGESGKGKSTLLQRIAMLWGSGKCKALTKFKFVFFLRLSRAQGGLFETLCDQLLDIPGTIRKQTFMAMLLKLRQRVLFLLDGYNEFKPQNCPEIEALIKENHRFKNMVIVTTTTECLRHIRQFGALTAEVGDMTEDSAQALIREVLIKELAEGLLLQIQKSRCLRNLMKTPLFVVITCAIQMGESEFHSHTQTTLFHTFYDLLIQKNKHKHKGVAASDFIRSLDHCGDLALEGVFSHKFDFELQDVSSVNEDVLLTTGLLCKYTAQRFKPKYKFFHKSFQEYTAGRRLSSLLTSHEPEEVTKGNGYLQKMVSISDITSTYSSLLRYTCGSSVEATRAVMKHLAAVYQHGCLLGLSIAKRPLWRQESLQSVKNTTEQEILKAININSFVECGIHLYQESTSKSALSQEFEAFFQGKSLYINSGNIPDYLFDFFEHLPNCASALDFIKLDFYGGAMASWEKAAEDTGGIHMEEAPETYIPSRAVSLFFNWKQEFRTLEVTLRDFSKLNKQDIRYLGKIFSSATSLRLQIKRCAGVAGSLSLVLSTCKNIYSLMVEASPLTIEDERHITSVTNLKTLSIHDLQNQRLPGGLTDSLGNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLKQLVFFDFSTKEFLPDPALVRKLSQVLSKLTFLQEARLVGWQFDDDDLSVITGAFKLVTA

SEQ ID NO: 3 - RNA, 19 nt, Artificial Sequence
  Artificially synthesized inhibitory RNA
gaggcacauacggcaguuu

SEQ ID NO: 4 - RNA, 19 nt, Artificial Sequence
  Artificially synthesized siRNA
ggacauuacauccacuuau

SEQ ID NO: 5 - RNA, 19 nt, Artificial Sequence
  Artificially synthesized siRNA
ggaacauccccgauuacuu

SEQ ID NO: 6 - RNA, 19 nt, Artificial Sequence
  Artificially synthesized siRNA
ggaugaacgugcuagaaca

SEQ ID NO: 7 - DNA, 3838 bp, Mus musculus
  CDS (217)..(3291)
ttctacagttgtctctcatggacaaagaagtttggattaagaaagaaagaaaattacttttaagacgacttttccttcgaaggcaactggattgcttggccaggagagccttgccaagagaagaggacaacagtatgatagtctttggccttcccatttggaaaaggaatattttctactgagatctaggaacgtgggacgctttgactcaccacaatgaactttataaggaacaacagacgagcccttattcaaaggatgggcttaacagttaccaagcaaatctgcgatgacctctttgcattgaacgttctcaacaatcaagaagctaatgtcatttactgtgagcccttggagcaggaagccgcccgaaagatcatccatatgactatgcagaagggctcagcggcctgcaacctctttcttaagagtcttgaaaactgggactattttgtgtatcaggacttaactggacaaaatctttcttatcaggtcacagaagaagacctgaatgttttggcccagaatttaaaggacttgtacaacagccctgcttttctgaacttctaccccctgggtgaagatatcgacataatttttaatctggagaaaaccttcacagaacctatcatgtggaagaaggaccatcgtcatcaccgtgtggagcagctgactttgggcagcctgctcgaggctctgaagagcccctgcctgattgaaggcgagtctggcaaagggaagtccaccctgctgcagagaatcgctatgctctgggcctctgggggctgcagggctctgaaggggttcagattagtcttcttcatccacctgagaagcgccagggggggactattcgaaacactgtacgatcagctcctgaacatacccgacttcatcagcaagccgaccttcaaggctctgctgctgaagctacacaaggaggtcctctttcttctcgatggttacaatgaattccatccccagaactgcccagaaattgaagccctgataaaggaaaaccatcgcttcaagaacatggtcattgtcaccaccaccacggagtgcctgaggcatatcagacatgttggcgccctgactgcggaggtgggagatatgaccgaagacagtgccaaagatctcatcgaggcagtgctggtacctgatcaggttgaacgcctgtgggcccaaatccaggagtccaggtgcctgagaaatctgatgaagacccctctcttcgtggtgatcacctgtgcaattcagatgggcagacaggaattccaagctcacacccaaaccatgctgttccaaaccttctacgacctcctgatacagaaaaacagccacagatatagaggtggagcttcaggtgattttgccaggagcctagactactgtggagacctggccctagaaggtgtgttcgcccacaaatttgattttgaacccgagcatgggtccagcatgaacgaggacgtcctggtgacaatagggctcctctgtaagtacacagctcagaggctgaagcccacgtataaattctttcataaatcatttcaggagtacacggcaggtcggagactcagcagtttgctgacgtccaaagagccagaggaggtgagcaaagggaacagctacttaaacaaaatggtttccatctctgacatcacatccctatatggcaatctgctcctctacacgtgtgggtcgtccacagaagcaaccagggcggtcatgaggcaccttgcaatggtttatcagcacggcagcctacaaggactttcagtcaccaagaggcctctctggaggcaggaatcaatccagagtctgagaaataccactgagcaagatgttctgaaagccatcaatgtaaattccttcgtagagtgtggcatcaatttgttctcagagagtatgtctaaatcagacctgagccaagaatttgaagctttctttcaaggtaaaagtttatacatcaactcagagaacatccctgactatttatttgacttctttgaatacttgcctaattgtgcaagcgcattggacttcgtgaagttggatttctatgaaagagctacagagtcacaggacaaggcagaagagaatgtccctggagttcacacagaagggccctcagaaacctacattccccccagggctgtgtctttgttcttcaactggaagcaggaattcaagactctagaggtcacactccgagatattaacaagttgaataagcaagatatcaaatatctggggaagatattcagctctgccaccaacctccggctgcatatcaagagatgtgcagccatggctggaagactcagctcagtcctcagaacctgcaagaacatgcataccctcatggtggaagccagtcccctcaccacggatgacgaacagtacatcacatctgtgacaggcctccagaacttaagtattcaccgcttgcacactcaacagctgccaggtggtctgattgacagcttgggtaatctgaagaacctcgagagactcatactggatgacatcaggatgaacgaggaagatgctaaaaacctagccgaaggcctacgaagcctgaagaagatgcgtttactccatttgactcatttgtctgacattggggaggggatggactacatagtcaagtctctctcagaagaatcctgtgatctccaagagatgaagttggtggcctgctgtctgactgcaaactctgtgaaagttctagcacagaatcttcacaatttgatcaagctgagcattcttgatatatcagaaaattacctggaaaaggatgggaatgaagctctacaggaactgatcggcaggcttggcgttctgggagagctcactacattgatgctgccttggtgctgggatgtgcacaccagcctgcccaagctgttgaagcagttggaggggaccccaggacttgccaaacttggattgaaaaactggagactcagagacgaagagattaaaagtttaggtgaatttctggagatgaatcctctgagagacttgcagcagttggatttagcggggcactgtgtgagcagtgacggatggctttacttcatgaatgtgtttgagaatctgaagcagttagtgttttttgactttagcactgaggagttcttaccggatgcagcactggtgaggaaacttagtcaagtgttatccaagttaactcttctgcaagaggtaaagctcacgggctgggagtttgatgactatgatattagcgctattaaaggcacctttaaactagtgactgcttaatgcacccgtgccaccaaatactccaggactccctcacttccagcaaatcattaaaaactacgcagaagtggggccacagagctggctcaggggttaagagcactggctgctcttccagaggacctgggattgattcgcaccacctacaaggtggctcacaaccatctgtaactccagttccaggggatccaacattttctgatctctatgggtaccacgcaggcaaaacactcatatacattaaataaaaatttaaaatgcttaaaaaaaaaatctcgaagaaggaagtcaaagaaggaaaagaagaggaattggcttgagaaacgagtttggtctcatctctccacacaggacaatctctgtgcccttcaggcgtgtttgtgttacttgaccccaagaatactgggtcaaaaaaccttgcgataccctgttcatctgtccttagtacagtacctggcctatctagactcaacaagtacttgataagtatgtgacaactaaattaccaagaaataaaattgtctagagaacagatttcatatatgct

SEQ ID NO: 8 - protein, 1024 aa, Mus musculus
MNFIRNNRRALIQRMGLTVTKQICDDLFALNVLNNQEANVIYCEPLEQEAARKIIHMTMQKGSAACNLFLKSLENWDYFVYQDLTGQNLSYQVTEEDLNVLAQNLKDLYNSPAFLNFYPLGEDIDIIFNLEKTFTEPIMWKKDHRHHRVEQLTLGSLLEALKSPCLIEGESGKGKSTLLQRIAMLWASGGCRALKGFRLVFFIHLRSARGGLFETLYDQLLNIPDFISKPTFKALLLKLHKEVLFLLDGYNEFHPQNCPEIEALIKENHRFKNMVIVTTTTECLRHIRHVGALTAEVGDMTEDSAKDLIEAVLVPDQVERLWAQIQESRCLRNLMKTPLFVVITCAIQMGRQEFQAHTQTMLFQTFYDLLIQKNSHRYRGGASGDFARSLDYCGDLALEGVFAHKFDFEPEHGSSMNEDVLVTIGLLCKYTAQRLKPTYKFFHKSFQEYTAGRRLSSLLTSKEPEEVSKGNSYLNKMVSISDITSLYGNLLLYTCGSSTEATRAVMRHLAMVYQHGSLQGLSVTKRPLWRQESIQSLRNTTEQDVLKAINVNSFVECGINLFSESMSKSDLSQEFEAFFQGKSLYINSENIPDYLFDFFEYLPNCASALDFVKLDFYERATESQDKAEENVPGVHTEGPSETYIPPRAVSLFFNWKQEFKTLEVTLRDINKLNKQDIKYLGKIFSSATNLRLHIKRCAAMAGRLSSVLRTCKNMHTLMVEASPLTTDDEQYITSVTGLQNLSIHRLHTQQLPGGLIDSLGNLKNLERLILDDIRMNEEDAKNLAEGLRSLKKMRLLHLTHLSDIGEGMDYIVKSLSEESCDLQEMKLVACCLTANSVKVLAQNLHNLIKLSILDISENYLEKDGNEALQELIGRLGVLGELTTLMLPWCWDVHTSLPKLLKQLEGTPGLAKLGLKNWRLRDEEIKSLGEFLEMNPLRDLQQLDLAGHCVSSDGWLYFMNVFENLKQLVFFDFSTEEFLPDAALVRKLSQVLSKLTLLQEVKLTGWEFDDYDISAIKGTFKLVTA

SEQ ID NO: 9 - RNA, 19 nt, Artificial Sequence
  Artificially synthesized siRNA
ggugaagauaucgacauaa

SEQ ID NO: 10 - RNA, 19 nt, Artificial Sequence
  Artificially synthesized siRNA
gugaagauaucgacauaau

SEQ ID NO: 11 - RNA, 19 nt, Artificial Sequence
  Artificially synthesized siRNA
gguucagauuagucuucuu

SEQ ID NO: 12 - RNA, 19 nt, Artificial Sequence
  Artificially synthesized siRNA
ggacuauucgaaacacugu

SEQ ID NO: 13 - RNA, 19 nt, Artificial Sequence
  Artificially synthesized siRNA
ggagauaugaccgaagaca

SEQ ID NO: 14 - RNA, 19 nt, Artificial Sequence
  Artificially synthesized siRNA
gcaaagggaacagcuacuu

SEQ ID NO: 15 - RNA, 19 nt, Artificial Sequence
  Artificially synthesized siRNA
ggcagccuacaaggacuuu

SEQ ID NO: 16 - RNA, 19 nt, Artificial Sequence
  Artificially synthesized siRNA
guaaauuccuucguagagu

SEQ ID NO: 17 - RNA, 19 nt, Artificial Sequence
  Artificially synthesized siRNA
gaacaucccugacuauuua

SEQ ID NO: 18 - RNA, 19 nt, Artificial Sequence
  Artificially synthesized siRNA
ggucacacuccgagauauu

SEQ ID NO: 19 - RNA, 19 nt, Artificial Sequence
  Artificially synthesized siRNA
gucacacuccgagauauua

SEQ ID NO: 20 - RNA, 19 nt, Artificial Sequence
  Artificially synthesized siRNA
gcacuggugaggaaacuua

SEQ ID NO: 21 - DNA, 4761 bp, Homo sapiens
  CDS (75)..(2264)
gccattttgtgcagtcgctgggaaggaaggagacgcctaaaccgcggcactgcccggtttgagcgtagccaaacctgcccaccggctttgtagccccgattctctgtgttttgctcccgtctccgacgagagaggcggcgacggtggcgtctgcgacgggagacagcgcgtcggagcgagagagcgctgcgcctgccgccgccccaacagcggaggcgccgccgccatcggtcgtcaccagaccggagccgcaggccctcccgagcccggccatccgtgccccgctcccagatctctatccttttgggaccatgcgcggaggaggctttggggaccgggaccgggatcgtgaccgtggaggatttggagcaagaggtggtggtggccttcccccgaagaaatttggtaatcctggggagcgtttgcgtaaaaaaaagtgggatttgagtgagctccccaagtttgagaaaaatttttatgtggaacatccggaagtagcaaggctgacaccatatgaggttgatgagctacgccgaaagaaggagattacagtgagggggggagatgtttgtcctaaacccgtgtttgccttccatcatgctaacttcccacaatatgtaatggatgtgttgatggatcagcactttacagaaccaactccaattcagtgccagggatttccgttggctcttagtggccgggatatggtgggcattgctcagactggctctgggaagacgttggcgtatctcctgcctgcaattgttcatattaaccaccagccatacttggaaaggggagatggcccaatctgtctagttctggctcctaccagagagcttgcccagcaagtacagcaggtggccgatgactatggcaaatgttctagattgaagagtacttgtatttatggaggtgctcctaaaggtccccagattcgagacttggaaagaggtgttgagatctgcatagccactcctggacgtctgatagatttcctggagtcaggaaagacaaatcttcgccgatgtacttaccttgtattggacgaagctgacagaatgcttgatatggggtttgaaccccagatccgtaaaattgttgaccaaatcaggcctgataggcagacactgatgtggagtgcaacctggccaaaagaagtaagacagcttgcagaggatttccttcgtgattacacccagatcaacgtaggcaatctggagttgagtgccaaccacaacatcctccagatagtggatgtctgcatggaaagtgaaaaagaccacaagttgatccaactaatggaagaaataatggctgaaaaggaaaacaaaacaataatatttgtggagacaaagagacgctgtgatgatctgactcgaaggatgcgcagagatggttggccagctatgtgtatccatggagacaagagtcaaccagaaagagattgggtacttaatgagttccgttctggaaaggcacccatccttattgctacagatgtagcctcccgtgggctagatgtggaagatgtcaagtttgtgatcaactatgactatccaaacagctcagaggattatgtgcaccgtattggccgaacagcccgtagcaccaacaagggtaccgcctataccttcttcaccccagggaacctaaaacaggccagagagcttatcaaagtgctggaagaggccaatcaggctatcaatccaaaactgatgcagcttgtggaccacagaggaggcggcggaggcgggggtggtcgttctcgttaccggaccacttcttcagccaacaatcccaatctgatgtatcaggatgagtgtgaccgaaggcttcgaggagtcaaggatggtggccggagagactctgcaagctatcgggatcgtagtgaaaccgatagagctggttatgctaatggcagtggctatggaagtccaaattctgcctttggagcacaagcaggccaatacacctatggtcaaggcacctatggggcagctgcttatggcaccagtagctatacagctcaagaatatggtgctggcacttatggagctagtagcaccacctcaactgggagaagttcacagagctctagccagcagtttagtgggataggccggtctgggcagcagccacagccactgatgtcacaacagtttgcacagcctccgggagctaccaatatgataggttacatggggcagactgcctaccaataccctcctcctcctccccctcctcctccttcacgtaaatgaaaccactcaagtggtagtgactccagcagacttaattacattttaaggaacactgtctttcctttttttttcctcttcgccttttctttttttttccttttttctttttttttttttaatttttccccccaaccatcgtgatttgtcttttcatgcagattagttagaattcactgccaggtttcttctgcccaccaaaatgatccagtctggaataacattttgtaaaaaaaaaaaaaatatatatatatatatatagctgactggaagagattaatttcttcccccaacttcttgcatgttgaagatatttgagctatttttcatctaaaagagtaaggtattaggcccttttgtgggagccccatgttttgtttttctgagttggtggggagggagggagggggagggctgaattgttttgcagaggaagatggcatctgtgctttaaatttctcattactgggttagaaaacaaagagggattgccctgcacattttcttttgtgcttttaaatgtttcttaagttggaacaggtttcctcgggcctgttttgactgattgctggagtgcatttgatagttaaaaattactaattggttttatttcccttcacactctgcctccccacttctccccccgttactgaaaaataaccattttagtgtcaggctagaaattgaattgctgagttttgtgtatcctttaaattaaaaaccacaagtgtttattgtagtggttaaactgtagcatctcagcatctgggtggaagctgcctatatttcttcccagtttaactggggaccatctgtgaaattaattttccatccagacagctgctgtgagcaaatgaacataaatgctcgctggaaatttactaaccagtttttatattgacctgcagtgtaaaaagcacatttaattataaacaatatattcaaaatgggcaaattttattttcaaatgcagtgtagagctagattaaaagcaactctttgccacctactctgcccttttggcaaagttaccttgaacaaagaatcttaagggtttattaagaactctttattttcttcataccctgttctctgcagtgctttctaacagcttctgggtgcagattttcttcggcatccttttgcactcagcttattacaggtaggtagtgcttaagaaaagtcatggaggactaaagcctaagtccttttcacttttcctccatctgaaggtaggtgagttcatcctcttcatggtaatgctgttttaccaagactttatagcagatggacccagaaagaattttctgctattgtgttcactacaacaggatagggacatcagacagccccagaaaccccttccagatctgatatgggactattaatttttatgctgttaattggtattcattcacaatgcagttgaagggggaaggctccactgcattctttggctaaggcctgaatgcttgctcatctgtaagatctatactcgaggttttgttttccttttaaaattctttagggagagagggatggtttctgaggggttctgaaagtatgattcaatgtgcaacatacaggtaggtcttcagcataagctgaaatatatgcatgtaaaaactttgacatctttttttttaattttccactttcttcttaactttacttctctttttgtcccccccccatcttacagaagttgaggccaagggagaatggtaggcacagaagaaacatggcaaactgctctgtgctttcaaaccaaagtgttccccccaaccccaaatttgtctaagcactggccagtctgttgtgggcattgttttctacaaccaaattctgggtttttttcttctttctttaaacatagaggtaccaccacaagggatgccctactctctcgcagctcttgaaagcatctgtttgagggaaaggtctctgggcaagcaagtggttatttggattgcttgcttccctttttccacctgggacattgtaatcataaaataacagtaaattccaaacctcaaaaactattatggcctgagcacagctgaaatctagcagagtttaactcttctgcctccatgtctgtcacttataattcaggttctgctgttggcttcagaacatgagcagaagaatcgttttatgctagttattgcattcatggttgaaactcaacttagggaaagggttccaatgtattaagcaatgggctgcttctccccaatcctccctaacaattcgttgtgtggacttctcatctaaaaggttagtggcttttgcttgggatcagtgctctctattgatgttcttgctggtctccagacacattcctgttgcattaagacttgaaagacttgtagatgtgtgatgttcaggcacaggatgctgaaagctatgttactattcttagtttgtaaattgtccttttgataccatcatcttgttttctttttgtaggtataaataaaaacactgttgacaataa

SEQ ID NO: 22 - protein, 729 aa, Homo sapiens
LPTGFVAPILCVLLPSPTREAATVASATGDSASERESAAPAAAPTAEAPPPSVVTRPEPQALPSPAIRAPLPDLYPFGTMRGGGFGDRDRDRDRGGFGARGGGGLPPKKFGNPGERLRKKKWDLSELPKFEKNFYVEHPEVARLTPYEVDELRRKKEITVRGGDVCPKPVFAFHHANFPQYVMDVLMDQHFTEPTPIQCQGFPLALSGRDMVGIAQTGSGKTLAYLLPAIVHINHQPYLERGDGPICLVLAPTRELAQQVQQVADDYGKCSRLKSTCIYGGAPKGPQIRDLERGVEICIATPGRLIDFLESGKTNLRRCTYLVLDEADRMLDMGFEPQIRKIVDQIRPDRQTLMWSATWPKEVRQLAEDFLRDYTQINVGNLELSANHNILQIVDVCMESEKDHKLIQLMEEIMAEKENKTIIFVETKRRCDDLTRRMRRDGWPAMCIHGDKSQPERDWVLNEFRSGKAPILIATDVASRGLDVEDVKFVINYDYPNSSEDYVHRIGRTARSTNKGTAYTFFTPGNLKQARELIKVLEEANQAINPKLMQLVDHRGGGGGGGGRSRYRTTSSANNPNLMYQDECDRRLRGVKDGGRRDSASYRDRSETDRAGYANGSGYGSPNSAFGAQAGQYTYGQGTYGAAAYGTSSYTAQEYGAGTYGASSTTSTGRSSQSSSQQFSGIGRSGQQPQPLMSQQFAQPPGATNMIGYMGQTAYQYPPPPPPPPPSRK

SEQ ID NO: 23 - RNA, 19 nt, Artificial Sequence
  Artificially synthesized siRNA
ggaagacguuggcguaucu

SEQ ID NO: 24 - RNA, 19 nt, Artificial Sequence
  Artificially synthesized siRNA
gauuuccuucgugauuaca

SEQ ID NO: 25 - RNA, 19 nt, Artificial Sequence
  Artificially synthesized siRNA
gaggccaaucaggcuauca

SEQ ID NO: 26 - RNA, 19 nt, Artificial Sequence
  Artificially synthesized siRNA
gcuaucgggaucguaguga

SEQ ID NO: 27 - RNA, 19 nt, Artificial Sequence
  Artificially synthesized siRNA
gcacaagcaggccaauaca

SEQ ID NO: 28 - DNA, 4772 bp, Mus musculus
  CDS (498)..(2456)
gaaccctacgccggttgcgcacattgccttgtttaaggtgggccgaccagggcccccatctccgtcaccgacgtaaagcggttccgagaatgtccgtggcgtcgcgcggagccctccagacgctgtttacgtagcgtctgggccgtgcgtagcgttaagttggagcggtctccgcggccgccgccattttgtgcagttgctgggaaggaagggagacgcctcaaccgtggcaccgcccggtttgagcgggaccagacctcccgactgtcctaacaaccccgattctctgagttgtgctcccgtctccgacgagagaggcggcgacggtggcgtctgcgacgggagacagcgcgtcggagcgagacagcgccgcgcctgccgccgccccaacagcggaggcgccgccgccgccatcggtcatcactagacccgagccgcaggccctcccgagctcggtcatccgtgccccgctcccagatctttatccgtttgggaccatgcgtggaggcggctttggggatcgggatcgtgacagggaccgtggagggtttggagcaagaggtggtagtgggcttccccctaagaagtttggtaatcctggggagcggttacgaaaaaagaagtgggatttgagtgaactccctaaatttgagaagaatttttatgttgagcatccagaagtagcaagactgactccgtatgaggttgatgagctacggcgtaagaaagagattacagtgagagggggagatgtttgtccaaaacctgtctttgccttccatcatgctaactttccacagtatgtgatggatgtgctgatggatcagcactttacagaacccactcccattcagtgccagggatttcctttggctcttagtggcagggatatggttggcattgcacagactggctctgggaagacattggcgtatttgctgcctgcgattgttcatataaaccaccagccatacttggaaagaggagatggtccaatttgtctagtgctggctcctaccagagagcttgcgcagcaggtgcagcaggtggctgacgattatggaaaatgctccaggttgaagagtacgtgcatttacggaggtgctcctaaaggtccccaaattcgagacttggaaagaggtgttgagatttgcatagccactcctgggcgcctaatagatttcctggagtcaggaaagacaaaccttcgccgatgtacttaccttgtgttggatgaggctgaccggatgcttgatatgggctttgagccccagatccgtaaaattgttgatcaaatcaggcctgaccggcagacactgatgtggagtgcaacctggccgaaggaagtgaggcagcttgcagaggatttcctgcgggactacacccagatcaatgtgggcaatctggagctgagtgccaaccacaacatcctacagattgtagatgtctgtatggagagtgaaaaagaccacaaattgatccagctgatggaggaaatcatggcggaaaaggaaaataagactataatatttgtggagacaaagaggcgctgtgatgacctcacacgaagaatgcgcagagatggttggcctgctatgtgtatccatggagacaagagtcaaccagaaagagattgggtacttaatgagttccgatctggaaaggctcctatcctcattgccacggatgtagcctcccgtgggctagatgtggaagatgtcaagtttgtcatcaactacgattatccaaacagctcagaggattatgttcaccgtattggccgaacggcccgcagcaccaacaagggcactgcctatactttctttaccccgggcaacctgaagcaggctagagagctgatcaaagtattggaagaggccaatcaagccatcaatccaaaattgatgcagcttgtggaccacagaggtggcggcggaggagggggtaagggaggccgctcacgataccggactacttcttcagccaacaatcccaatctgatgtatcaggacgagtgtgaccggaggcttcgaggggtcaaggatggtggccggagagattctacaagctacagggatcgtagtgaaaccgatagagccagttatgctaatggcagtggctatggaagcccaaattctgcctttggggcacaagcaggccaatacacctatgctcaaggcacctatggggcagctgcctatggcaccagtggctacacggcgcaggagtatgctgctggcacttacggggcgagcagcactgcctcagcagggaggagctctcagagctccagccagcagtttagtgggataggccgatctgggcagcagccacagccactgatgtcacagcagtttgcacagcctccaggagctaccaatatgataggctacatggggcagactgcttaccagtaccctccccctccccctccccctcctccatctcgcaaatgaaactgcatggcaggagtgactcgtgcagatgaattacacatctaaaggaacactgtcttttacccttctagaccttttcatttttattttttcttttctcaaccattacggtttatctttttttaatgcagatagttaaaatttcactgccaggtttcttctgcccacccaaaagatccagtctataattacagattttgtaagaaaaaatatatatacacataactgactggaaagaattaatttctttcccccagcttaatgcatgttgaggatattgagctatttttcatcttaaaggtattaggcacttttgttggagcctgtttatccatgggagggtgggtagggaaggagggactgctctgcaggaagtggtatctgttctttagactgctctccttagggaggagccaagagggatttccatgcacactgcccatggtgggtttaagttactgaagttcgattactcaggcctgatgaatttttaagactgctttgttggtcatttggctgatggcttcttatgtgcattcaacagttaaaaattcctaatcgattctgtcatcatgccccctccccacttgttcctgcagttattaaatattaaccaggttagtgttgagttagaaattacatttctgagttcaagcgttgtctttttaagtttaaaaaactataaagggttgtttttactaggtagactggcatctgggtagaggctgcctgggatttttatccccacataaccaggggccatctgaaagtcattttctagccagatcagccactgagcggtaagcatcagcgctcagcagagccttccgagcagcatttagaaagcacctttgggaatgatggactgcaagggagtgggcggatcttcctcttaaacagtattcttagagtaggattgacctcatgggctgctctgcccacttggtcaggctctcctgagcaggactgctaaagagcttgcctcctcaaagcctggtctctgaggtgctttctaacagctggaggcacttgttctgggctttcctttgccctcagtttttcacaggtaggtagtagccatggagggctgaagcccaggctctccttactctctggagtgtgaaggtaggggtgagctcgtctcacccctttattaaatgctcctggaacttggaagagcagcagatgaagttggaaagaaaattctgctcttatcacctaaacaggacaggagagcagatgccccagcgcctctagtagatggtagtggtggggctagtgtgtggctgttacctgggccgctcaaattcattccagggaggaggctccactgcattctttggctgtgccctgagggtgcctgctcctttgtacggtaacagtcaattttgaggtttctttcaggaagagaaagggatggttttgaggggctcagaaaataggattcagtgtgtaacataacaggtaggttgtcagcatgcttggcattcttctgttttccttaccgttttacagactatgaagctgaggggggatggtaggcacagatgtgtggcaaccctgctctgtgctctgaaaccaaagtgtgtcctgtcttgtctgtctctcaccctgccaaaagcaagtgtctaatcactgatgctgaccagcctgttacagatactttctacaaccaaattcagatttattttttattaagcattataccatgggctgtcctgtgctcctgtcactcttcaaaaacaccccttctattactgcctccggggatgaagcagtagctaggttgcttaacctcccctctccagcagaggcataatccagataagtagcagatgtcatatttcataagctcttttggcctgggtacagcagaaaactagcagttttcctacttggcgcaagtgacttgtgcctccttgtctgtcctttgtcagcacaggtgcaggcaaacccttccagctgggttaattgccgtttatggtttactcacttagggaaagggcttaggtgttaggctgtgggctgcttctccctaaccatccattgtgcagacttctcatctaaaaaggttggtggcttttgcttgggatcagtgctctgctaacgcccttgctggtctctccacacattcctgtcattgagacttgaattgtaggtgtgatgttatgcacaggatgctcagagctatgttactactattcttagtttgtaaattgtccttttgataccatcttgttttcttttgtaggtataaataaatacttgacaataaaggtgtgaattttttattacattaaaacttaaaaagagttt

SEQ ID NO: 29 - protein, 652 aa, Mus musculus
MRGGGFGDRDRDRDRGGFGARGGSGLPPKKFGNPGERLRKKKWDLSELPKFEKNFYVEHPEVARLTPYEVDELRRKKEITVRGGDVCPKPVFAFHHANFPQYVMDVLMDQHFTEPTPIQCQGFPLALSGRDMVGIAQTGSGKTLAYLLPAIVHINHQPYLERGDGPICLVLAPTRELAQQVQQVADDYGKCSRLKSTCIYGGAPKGPQIRDLERGVEICIATPGRLIDFLESGKTNLRRCTYLVLDEADRMLDMGFEPQIRKIVDQIRPDRQTLMWSATWPKEVRQLAEDFLRDYTQINVGNLELSANHNILQIVDVCMESEKDHKLIQLMEEIMAEKENKTIIFVETKRRCDDLTRRMRRDGWPAMCIHGDKSQPERDWVLNEFRSGKAPILIATDVASRGLDVEDVKFVINYDYPNSSEDYVHRIGRTARSTNKGTAYTFFTPGNLKQARELIKVLEEANQAINPKLMQLVDHRGGGGGGGKGGRSRYRTTSSANNPNLMYQDECDRRLRGVKDGGRRDSTSYRDRSETDRASYANGSGYGSPNSAFGAQAGQYTYAQGTYGAAAYGTSGYTAQEYAAGTYGASSTASAGRSSQSSSQQFSGIGRSGQQPQPLMSQQFAQPPGATNMIGYMGQTAYQYPPPPPPPPPSRK

SEQ ID NO: 30 - RNA, 19 nt, Artificial Sequence
  Artificially synthesized siRNA
ggaucagcacuuuacagaa

SEQ ID NO: 31 - RNA, 19 nt, Artificial Sequence
  Artificially synthesized siRNA
ggaagacauuggcguauuu

SEQ ID NO: 32 - RNA, 19 nt, Artificial Sequence
  Artificially synthesized siRNA
guccccaaauucgagacuu

SEQ ID NO: 33 - RNA, 19 nt, Artificial Sequence
  Artificially synthesized siRNA
gcugaccggaugcuugaua

SEQ ID NO: 34 - RNA, 19 nt, Artificial Sequence
  Artificially synthesized siRNA
gcucagaggauuauguuca

SEQ ID NO: 35 - DNA, 4187 bp, Homo sapiens
  CDS (764)..(3874)
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 36 - protein, 1036 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 37 - DNA, 3945 bp, Mus musculus
  CDS (139)..(3240)
tttcccctttatttgtacccaaggctgctatctggaggaacttttcttccatggctcaggacatacgtctggatcaagctaagagaactttctgtgtggacctaagccccgagaccctcgaaagggctgctgctgaagatgacgagtgtccgttgcaagctggctcagtatctagaggaccttgaagatgtggacctcaagaaattcaaaatgcatttggaagattacccgcccgagaaaggctgtatcccagtccccaggggccagatggagaaggcagatcacttggatctagccacactcatgattgacttcaatggcgaggagaaggcctgggccatggctgtgtggatctttgctgcgatcaacaggcgagacctctgggaaaaagctaagaaggaccagccagagtggaatgacacgtgtacatcacattcctctatggtatgccaggaggacagccttgaagaagagtggatgggtttgctgggatatctctcccgcatctccatttgtaaaaagaagaaagattactgtaagatgtacagacgacatgtgagaagcaggttctactctatcaaggacaggaacgcgcgtctaggtgagagtgtggacctcaacagtcgctacacgcagctccaactggtcaaggagcatccaagcaagcaggagcgggagcatgaactcctgaccatcggccggactaaaatgcgggacagccccatgagttcccttaagctggagctgctgtttgagcccgaggacgggcactcggagcctgtgcacacagtggtgttccagggagcagcaggcatcgggaaaaccatcctagccaggaagattatgttggactgggcactgggaaagctcttcaaagacaaatttgactatttgttctttatccactgccgagaggtgagcctcaggacgccaaggagtctagcagacctgattgtcagctgctggcctgacccaaacccaccagtgtgcaagatcctgcgcaagccttccaggatcctcttcctcatggatggctttgatgagctacaaggggcctttgacgagcacattggggaggtctgcacagactggcaaaaggctgtgcggggagacattctgctaagcagcctcatccgaaagaaactgctgcccaaggcctctctgctcataacgacgaggccggtagccttggagaaactgcagcatctcctggaccacccccgccatgtggagatcctaggtttctctgaggccaaaaggaaggagtatttctttaagtatttctccaacgagctgcaggcccgggaggccttcaggctgatccaagagaatgaggtcctctttaccatgtgcttcatccccctggtctgctggattgtgtgcacggggctaaagcaacagatggagaccgggaagagcctggcccagacctccaagaccactacggccgtctacgtcttcttcctttccagcctgctgcaatcccgggggggcattgaggagcatctcttctctgactacctacaggggctctgttcactggctgcggatggaatttggaaccagaaaatcctatttgaggagtgtgatctgcggaagcacggcctgcagaagactgacgtctccgctttcctgaggatgaacgtgttccagaaggaagtggactgcgagagattctacagcttcagccacatgactttccaggagttcttcgctgctatgtactatttgctggaagaggaggcagagggggagaccgtgaggaaaggaccaggaggttgttcagatcttctgaaccgagacgtgaaggtcctactagaaaattacggcaagtttgaaaaaggctatctgatttttgttgtccgattcctctttggccttgtaaaccaggagagaacctcttatttggagaagaaactaagttgcaagatctctcagcaagtcagactggaactactgaagtggattgaagtgaaagccaaggccaagaagctgcagtggcagcccagccaactggaactgttctactgcctgtacgagatgcaggaggaagactttgtgcagagtgccatggaccactttcccaaaattgagatcaacctctctaccagaatggaccacgtggtttcctccttttgtattaagaactgtcatagggtcaaaacgctttccctgggtttttttcacaactcgcccaaggaggaagaagaagagaggagaggaggtcgacccttggaccaggttcagtgtgttttcccagacactcatgttgcctgttcttccagactggtgaactgctgcctcacttctagcttctgccgtggtctcttctcaagtctaagcaccaaccggagcctcactgaactggacctcagtgacaatactctgggagacccgggcatgagggtgctgtgtgaggcactccagcacccaggctgtaacattcagagactgtggttggggcgctgcggactgtcccatcaatgctgcttcgacatctcctctgtcctgagcagcagccagaagctggtggagctggacctcagtgacaatgccctgggggactttggaatcagattgctgtgtgtgggactgaagcacctgctctgcaacctccagaaactgtggttggtgagctgctgtctcacatccgcgtgttgtcaggatctcgcattggttctgagctccaaccattctctgaccagactgtacattggagaaaatgccttgggagactcaggagtccaagttttgtgtgaaaagatgaaggacccacagtgtaacttgcagaagctggggttggtgaattccggccttacttcaatctgttgttcagctctgacctctgtgctcaaaaccaaccagaacttcacacacctctatctacgaagcaatgcccttggagacacaggactcaggctcctctgtgaggggcttctgcacccggactgtaaactacagatgctggaattagacaactgcagcctcacctcacacagctgctggaatctctccacaattctgacccacaaccacagccttcggaagctgaacctgggcaacaatgatcttggcgatctgtgcgtggtgaccctctgtgaggtgctgaaacagcagggctgcctcctgcagagcctacagttgggtgaaatgtacttaaatcgtgaaacaaaacgtgccttagaagcgctccaggaagaaaagcctgagctgactatagtcttcgagatttcctggtaggcgtggaagcaggaccaccaggtgcctcggtcctgccccaagtcctgccccaagccccagtgcgcactgctcttcactgctatcaagccctccttcaccatcaggatcacagccgaggctcttctggtatagggtctggagcaaaggcttgtgtgggaccaaatatttttcctcacatcgataacgtgaaactgccagaggctgcccttcccatcatatcctcagtgggcaaggtgttccctcttggtgacttcatggaaacagcttcaagaaaacgccttttctgtcctcccccgccctcctcttactcctgcccctcctcctcctcctcctcccctccccccccctcctcctccgcttctccccccacctgtctttctctctctgggcctctggttttttgacctttgcccataccttcagtcttgtcttcctgttaactgaccatcccgcataaggagctgcccgtgggctagatggaaggtttgtggcagcctctcagctacattgtttgtttttattttttaatagttatgatttctctttagctacctgaaaactcagagatttataaaacccatttttgtatttattgtatgtttgtactgctttcttaatttaaaaatgtatctagaattcttttaagttatttatccaaactactaaaaataaatcagtttacacatttaaaaaaaaaaaaaaaaa

SEQ ID NO: 38 - protein, 1033 aa, Mus musculus
MTSVRCKLAQYLEDLEDVDLKKFKMHLEDYPPEKGCIPVPRGQMEKADHLDLATLMIDFNGEEKAWAMAVWIFAAINRRDLWEKAKKDQPEWNDTCTSHSSMVCQEDSLEEEWMGLLGYLSRISICKKKKDYCKMYRRHVRSRFYSIKDRNARLGESVDLNSRYTQLQLVKEHPSKQEREHELLTIGRTKMRDSPMSSLKLELLFEPEDGHSEPVHTVVFQGAAGIGKTILARKIMLDWALGKLFKDKFDYLFFIHCREVSLRTPRSLADLIVSCWPDPNPPVCKILRKPSRILFLMDGFDELQGAFDEHIGEVCTDWQKAVRGDILLSSLIRKKLLPKASLLITTRPVALEKLQHLLDHPRHVEILGFSEAKRKEYFFKYFSNELQAREAFRLIQENEVLFTMCFIPLVCWIVCTGLKQQMETGKSLAQTSKTTTAVYVFFLSSLLQSRGGIEEHLFSDYLQGLCSLAADGIWNQKILFEECDLRKHGLQKTDVSAFLRMNVFQKEVDCERFYSFSHMTFQEFFAAMYYLLEEEAEGETVRKGPGGCSDLLNRDVKVLLENYGKFEKGYLIFVVRFLFGLVNQERTSYLEKKLSCKISQQVRLELLKWIEVKAKAKKLQWQPSQLELFYCLYEMQEEDFVQSAMDHFPKIEINLSTRMDHVVSSFCIKNCHRVKTLSLGFFHNSPKEEEEERRGGRPLDQVQCVFPDTHVACSSRLVNCCLTSSFCRGLFSSLSTNRSLTELDLSDNTLGDPGMRVLCEALQHPGCNIQRLWLGRCGLSHQCCFDISSVLSSSQKLVELDLSDNALGDFGIRLLCVGLKHLLCNLQKLWLVSCCLTSACCQDLALVLSSNHSLTRLYIGENALGDSGVQVLCEKMKDPQCNLQKLGLVNSGLTSICCSALTSVLKTNQNFTHLYLRSNALGDTGLRLLCEGLLHPDCKLQMLELDNCSLTSHSCWNLSTILTHNHSLRKLNLGNNDLGDLCVVTLCEVLKQQGCLLQSLQLGEMYLNRETKRALEALQEEKPELTIVFEISW

SEQ ID NO: 39 - DNA, 1436 bp, Homo sapiens
  CDS (32)..(1246)
cagtcacacaagaagggaggagagaaaagccatggccgacaaggtcctgaaggagaagagaaagctgtttatccgttccatgggtgaaggtacaataaatggcttactggatgaattattacagacaagggtgctgaacaaggaagagatggagaaagtaaaacgtgaaaatgctacagttatggataagacccgagctttgattgactccgttattccgaaaggggcacaggcatgccaaatttgcatcacatacatttgtgaagaagacagttacctggcagggacgctgggactctcagcagatcaaacatctggaaattaccttaatatgcaagactctcaaggagtactttcttcctttccagctcctcaggcagtgcaggacaacccagctatgcccacatcctcaggctcagaagggaatgtcaagctttgctccctagaagaagctcaaaggatatggaaacaaaagtcggcagagatttatccaataatggacaagtcaagccgcacacgtcttgctctcattatctgcaatgaagaatttgacagtattcctagaagaactggagctgaggttgacatcacaggcatgacaatgctgctacaaaatctggggtacagcgtagatgtgaaaaaaaatctcactgcttcggacatgactacagagctggaggcatttgcacaccgcccagagcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcctgccgtggtgacagccctggtgtggtgtggtttaaagattcagtaggagtttctggaaacctatctttaccaactacagaagagtttgaggatgatgctattaagaaagcccacatagagaaggattttatcgctttctgctcttccacaccagataatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaaataaggaaactgtatgaatgtctgtgggcaggaagtgaagagatccttctgtaaaggtttttggaattatgtctgctgaataataaacttttttgaaataataaatctggtagaaaaatgaaaacttgtcctcatttttctcccacactgaagaaacagggactggaacttagagtgactaaggaattt

SEQ ID NO: 40 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 41 - DNA, 1533 bp, Mus musculus
  CDS (201)..(1409)
cttccaagtgttgaagaagaatcatttccgcggttgaatccttttcagacttgagcatttaaacctaactttaattagggaaagaaacatgcgcacacagcaattgtggttatttctcaatctgtattcacgccctgttggaaaggaactaacaatatgctttcagtttcagtagctctgcggtgtagaaaagaaacgccatggctgacaagatcctgagggcaaagaggaagcaatttatcaactcagtgagtatagggacaataaatggattgttggatgaacttttagagaagagagtgctgaatcaggaagaaatggataaaataaaacttgcaaacattactgctatggacaaggcacgggacctatgtgatcatgtctctaaaaaagggccccaggcaagccaaatctttatcacttacatttgtaatgaagactgctacctggcaggaattctggagcttcaatcagctccatcagctgaaacatttgttgctacagaagattctaaaggaggacatccttcatcctcagaaacaaaggaagaacagaacaaagaagatggcacatttccaggactgactgggaccctcaagttttgccctttagaaaaagcccagaagttatggaaagaaaatccttcagagatttatccaataatgaatacaaccactcgtacacgtcttgccctcattatctgcaacacagagtttcaacatctttctccgagggttggagctcaagttgacctcagagaaatgaagttgctgctggaggatctggggtataccgtgaaagtgaaagaaaatctcacagctctggagatggtgaaagaggtgaaagaatttgctgcctgcccagagcacaagacttctgacagtactttccttgtattcatgtctcatggtatccaggagggaatatgtgggaccacatactctaatgaagtttcagatattttaaaggttgacacaatctttcagatgatgaacactttgaagtgcccaagcttgaaagacaagcccaaggtgatcattattcaggcatgccgtggagagaaacaaggagtggtgttgttaaaagattcagtaagagactctgaagaggatttcttaacggatgcaatttttgaagatgatggcattaagaaggcccatatagagaaagattttattgctttctgctcttcaacaccagataatgtgtcttggagacatcctgtcaggggctcacttttcattgagtcactcatcaaacacatgaaagaatatgcctggtcttgtgacttggaggacattttcagaaaggttcgattttcatttgaacaaccagaatttaggctacagatgcccactgctgatagggtgaccctgacaaaacgtttctacctcttcccgggacattaaacgaagaatccagttcattcttatgtacctatgctgagaatcgtgccaataagaagccaatacttccttagatgatgcaataaatattaaaataaaacaaaacaaaaaaaaaaaaaaaaaaaaa

SEQ ID NO: 42 - protein, 402 aa, Mus musculus
MADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 43 - DNA, 1296 bp, Homo sapiens
  CDS (50)..(1183)
actctgaggctctttccaacgctgtaaaaaaggacagaggctgttccctatggcagaaggcaaccacagaaaaaagccacttaaggtgttggaatccctgggcaaagatttcctcactggtgttttggataacttggtggaacaaaatgtactgaactggaaggaagaggaaaaaaagaaatattacgatgctaaaactgaagacaaagttcgggtcatggcagactctatgcaagagaagcaacgtatggcaggacaaatgcttcttcaaaccttttttaacatagaccaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctagaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattgaaaatggaagccacaagcagcccagccctccttaatcaacttcaaggagcaccttcattagtacagcttgcatatttaacattttgtatttcaataaaagtgaagacaaacgaa

SEQ ID NO: 44 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 45 - RNA, 57 nt, Artificial Sequence
  Artificially synthesized inhibitory RNA
ccggagacuauguaaagaaagagcucucgagagcucuuucuuuacauagucuuuuuu

SEQ ID NO: 46 - RNA, 19 nt, Artificial Sequence
  Artificially synthesized inhibitory RNA
guguagauguagaagagaa

SEQ ID NO: 47 - RNA, 19 nt, Artificial Sequence
  Artificially synthesized inhibitory RNA
ccuagaggaagaugcuguu

SEQ ID NO: 48 - RNA, 19 nt, Artificial Sequence
  Artificially synthesized inhibitory RNA
cuacacugugguugacgaa

SEQ ID NO: 49 - RNA, 19 nt, Artificial Sequence
  Artificially synthesized inhibitory RNA
ccauagaacgagcaaccuu

SEQ ID NO: 50 - RNA, 19 nt, Artificial Sequence
  Artificially synthesized inhibitory RNA
cagcagaaucuacaaauau

SEQ ID NO: 51 - RNA, 19 nt, Artificial Sequence
  Artificially synthesized inhibitory RNA
cggaugugcugcuuuauga

SEQ ID NO: 52 - DNA, 1443 bp, Mus musculus
  CDS (60)..(1181)
actttcattttactctgtcaagctgtcttcacggtgcgaaagaactgaggctttttctcatggctgaaaacaaacaccctgacaaaccacttaaggtgttggaacagctgggcaaagaagtccttacggagtacctagaaaaattagtacaaagcaatgtactgaaattaaaggaggaagataaacaaaaatttaacaatgctgaacgcagtgacaagcgttgggtttttgtagatgccatgaaaaagaaacacagcaaagtaggtgaaatgcttctccagacattcttcagtgtggacccaggcagccaccatggtgaagctaatctggaaatggaggaaccagaagaatcattgaacactctcaagctttgttcccctgaagagttcacaaggctttgcagagaaaagacacaagaaatttacccaataaaggaggccaatggccgtacacgaaaggctcttatcatatgcaatacagagttcaaacatctctcactgaggtatggggctaactttgacatcattggtatgaaaggccttcttgaagacttaggctacgatgtggtggtgaaagaggagcttacagcagagggcatggagtcagagatgaaagactttgctgcactctcagaacaccagacatcagacagcacattcctggtgctaatgtctcatggcacactgcatggcatttgtggaacaatgcacagtgaaaaaactccagatgtgctacagtatgataccatctatcagatattcaacaattgccactgtccaggtctacgagacaaacccaaagtcatcattgtgcaggcctgcagaggtgggaactctggagaaatgtggatcagagagtcttcaaaaccccagttgtgcagaggtgtagatctacctaggaatatggaagctgatgctgtcaagctgagccacgtggagaaggacttcattgccttctactctacaaccccacatcacttgtcctaccgagacaaaacaggaggctcttacttcatcactagactcatttcctgcttccggaaacatgcttgctcttgtcatctctttgatatattcctgaaggtgcaacaatcatttgaaaaggcaagtattcattcccagatgcccaccattgatcgggcaaccttgacgagatatttctacctctttcctggcaactgagaacaaagcaacaagcaactgaatctcatttcttcagcttgaagaagtgatcttggccaaggatcacattctattcctgaaattccagaactagtgaaattaaggaaagaatacttatgaattcaagaccagcctaagcaacacagtgggattctgttccatagacaagcaaacaagcaaaaataaaacaaaaaaaaaatttaccaaaagagaaatttgttttatttatttgtgtacataaataaaaagaaagcaaataatt

SEQ ID NO: 53 - protein, 373 aa, Mus musculus
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEANGRTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

SEQ ID NO: 54 - DNA, 3201 bp, Homo sapiens
  CDS (117)..(1685)
gcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcacacctgtaatcccagctctttggaggccgaggcaggcggatcacttgaggtcaggagtttgagaccagcctgaccaacatggtgaaactccatctctactaaaaatataaaaattagccgggcatggtgatgcatgcctgtaatcccagctactcgggaggcttagacatgagaatcacttgaacccaggaggtggaggttgcagtgagtcaagatggcaccactgcactccatcctgggtgacagagcaagacttcctctcaaaaaataaataaataaataagaaaaataaattaggaaattattaaaataattttttagaaaagcaacaaaataacaaaaattagtgactgtaataattggaatgtttgaaatttgtcacagcaataattgactaaaatgaatgtaaataattattcagttttcagtttgttgtaataaactctaatcagtataaaggattgactatcctaggaaaaagattagtcaaggaataagaagcaaaagtagtatttgaaatctaaaaacattaccatgtttgctttatcatgcaggcaaataaataaataaatgacatttttaggttggaataaaaaaagataaagtacatattgccttaattaaattcatcaagttcttactgaaaaacagctctccgccgtgcctggccaggaaatacataattttttttttttttttttagatggagtctcgctctgtcgcccaggctggagtccagtggcatgatcttggctcactgcaagctctgcttcctgggttcatgccattctcctgcctcagccttccgagtagctgggactacaggtgcccgccaccacatccggctaattttttgtatttttagtaaagatggggtttcaccatgttagccaggatggtctcgatctccttaccttgtgatccgcccgccttggcctcccaaagtgctgggattacaggtgtgagccaccacgcctggctgaaatacataatcttaaaagaaaacataagatactttattttaatatacgtgactaaatgtaaaacctaacttattttctgttatctatttatttttactttcagtaacactttttttattttaggtagcattcagcctagaggcaactgctgtttgttaaatatttcctgttcatatattttgcacattttcttatgggttagttttcttctcattgttttgggaagttcttaatatatttggggtatttatctttcattcgttgtctgtgtaacaaataacttctgccatatgggttgtctgcacattttttggtgtcttttagtaaacaaggtttttttgttttgtattgttttgtttattgtaaagatttttaaattttaatggagttgatttcttttctcattcaagcttttgagaataaattggagttgaattttt

SEQ ID NO: 55 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 56 - RNA, 59 nt, Artificial Sequence
  Artificially synthesized inhibitory RNA
ccggcuuugauaacugcgugacauacucgaguaugucacgcaguuaucaaaguuuuuug

SEQ ID NO: 57 - RNA, 59 nt, Artificial Sequence
  Artificially synthesized inhibitory RNA
ccggccugcuguaacacuucuuauucucgagaauaagaaguguuacagcagguuuuuug

SEQ ID NO: 58 - RNA, 47 nt, Artificial Sequence
  Artificially synthesized inhibitory RNA
agaagaaacauggcggcuauccuucucucacaucgaaaaggaaauuu

SEQ ID NO: 59 - DNA, 4090 bp, Mus musculus
  CDS (160)..(1683)
gacttcacgcgtgctcctgcgcctgctcgcggcggatactgaccggctacgttcccgccatatatttaagtttcatttccagcggccactgagagttccccttttcgcggcctttcttcgaagtcgatttattctttcccggaccgaagagcggcagatatggaagatccgcgtagaaggacgacggcgccacgcgctaagaagccgtccgcgaagcgcgccccgacgcagcccagcaggaccagggcccacgcggaaagctgcggcccgcaaaggggggctcgatcgcggcgggcggagcgtgacggggacaccacggagaagccacgtgccccagggccccgagtgcatccagcaagggccactgagctcaccaaagatgcacagccctcggccatggacgcggcaggagccaccgcgcggcctgccgtccgggtgccccagcagcaggccatcctggatccggagctgcccgccgtacgggagccccagccgcccgcggatcccgaggcgcggaaagtcgtaaggggacctagccacagaaggggcgcgcgctccaccgggcagcccagagcgccgcgagggtccaggaaggaaccggacaagctaaagaaggtgctggacaaattgagattgaaacgcaaagatatctcggaggcggccgagacggtgaataaagttgtggaacgcctgctgcgcagaatgcagaaacgggagtcggagttcaaaggtgtggagcagctgaacactggcagctactatgaacatgtgaagatttctgctcctaatgaatttgatgttatgtttaaactggaagtccccaggattgagctacaagaatattatgaaacaggtgctttctatcttgtgaaattcaaaagaattccacgaggaaatccgctgagtcatttcttagaaggggaagtattatcagctaccaagatgctgtcaaagtttaggaaaatcattaaagaagaagttaaagaaatcaaagatatagatgtcagtgtggagaaggaaaaaccaggaagccctgctgtaacacttcttatcaggaaccctgaagaaatctctgtggatataattctggctttggagtcaaaaggcagctggcctattagtaccaaagaaggactacctattcaaggctggctgggcacaaaagtgaggaccaatctaagacgagagccgttttatctcgtacccaagaatgcaaaggatggaaatagttttcaaggagagacctggcgcctctctttctctcacactgaaaagtacattttgaataatcacgggatagagaaaacatgctgtgaatcttccggagcaaaatgctgcagaaaagaatgtttaaaattaatgaaataccttttggaacagttgaaaaaagagtttcaagagctggatgcattctgttcctaccatgtgaaaactgccatctttcacatgtggacccaggacccgcaggacagtcagtgggaccccaggaacctcagctcctgcttcgataagttgttagcattctttcttgagtgcctcaggacagagaaactggatcattattttattccaaagttcaatctattctctcaagaactaattgaccgaaaaagtaaagaatttctatcgaagaaaattgaatatgaaagaaataatgggtttccaatttttgacaagctttgaaactgtatttgtgttatatgttataatgtgtgtctgtggggttttaggtcagatgtcgattgatgccaggcgtcttcttcagtcatacttcacttttgggggtggtgggaagagtggtgatttcaagacatggtttctgtgtagtcctggctgtcttgtaactccctctgtagaccaggctggtcttgaacaaagagatctgcctgcttctgccattggaatgcagggattaaaggcctatgccaccattaggtggcacacttcacttttttgagctcagggaggtgtccgggctgattagccagtgaactcctgggattcagctgcctctgctgccccaactctgggatgacagatggacatcacctattcagcttttacattgttgctagcggtctcaactcaagtcctcacgcttgcaaagcaagtactatagtgactgagccatcttcccagcctgacattgccttctgaaatcaaattgggttaaagtgatgtaagtcacttgcctgtagtttcagtacttgagaacttttctttgtttgggttttactgctatgaacagacaccatgaccaaggcaacttttataagggccagcatttcattgaggctggtttataggttcggagatttagtctgttgtcatcatagcaggaagcgtggcagcatccaggagggcagagcactggagaagtagctgagagttctacatcttcatccaaaggaagcgggtagcagactgccacccacatggccaagaggagagtcttaaaagcccacctctatggcggtgacacacttcctccaacagtgttacacctactccgataaagccactcgtcctcgtagtgccacaccctgcgcggagcatattcaaaccaccatggaagtggaggcagaaaaatagtccagtgtggttcttagctatatttttagttcaggcatagcctgaggtacacgaaacctgcttaaaacaaagttcaatcgttaacagcacttattgctcttccggagggcctgggttggttcccagcatccagtggcaggcacaactgtaactctggttccagggaatctagtttgaccacgacacacacacacacacactgacactattttttaagagcacaaaaagaaaatgaaattataatctgaccccataatatgataagtgtgctagcacttcattataatcctggcacttgagaagctgagatgtgacggttgccatgaatgcaaggccagcctgggatataaaatacaatcttgtttaaaaaaacaaaacaaaacaccaagagccaaaagccgtccctaggagtcactgtgaatccctatgagtttggcctgggtctgctcacctgtcctgactcaaaagggtacattttctttttctatttttcctctttaggattgtcagaatttacggaggatttttttttggtatgttgtgcttcttgacgctctcatgaaatctgcccccctccccaactccttgacacacaccccatatggctagtgtgtctattaaatggctgatgttctctttgagtacaaaataagaacccccaaaatgtgaataaaataattcaaaaaaaaaaaaaaaaaaaaaaagctgggcagcatgcctttaatcccagcacttgggaggcagaggcaggaggatttctgagttcgaggccaccctggtctacaaagtgagttccaggacagccagagctacacacagaaaccctgtctcgaaaagaaaaaaaaaaattcagatgttcatagcactgactaaaatggatataattatttaatactcagtttctcataattcactcacacataaagagtataacaaaaatgaaatactaaagataaaaacaaaaaccttgccgtattagaaagggaaaacaaataaaaactttccccagtgagactcattaagagcttaatgagtatactcacctagtagaatccgcagaaggttgtgagagattgtattggaaatagttgatttagaatttgttcaaacacaagaaatgcactgccgcctcctctgtcctaaggtagaagggaggattgtgtactccgtcagctctccctcaggtactttatcatgtgtgcaggagcatgtaatttatttttatctttattctatacgtgtttttacaaaagcaatgatacagcgcagcgagatgggtgtgggacagaggatactctcgtctgcatccaagcagcgttccccaggggcagctgatgctcatttcctctttttgagtactttgttgcatgggttgttgcaggagccttttccagtggtctgtagctgttccctgtgtaacaaataatgtctgccatgtatgttgtcttcactttttaaaagtatctctcaataaataagatttaaatgttttaatgta

SEQ ID NO: 60 - protein, 507 aa, Mus musculus
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 61 - DNA, 2170 bp, Homo sapiens
  CDS (303)..(1442)
gttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagatta

SEQ ID NO: 62 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 63 - RNA, 59 nt, Artificial Sequence
  Artificially synthesized inhibitory RNA
ccggccaacauucgcuuccuggauacucgaguauccaggaagcgaauguugguuuuuug

SEQ ID NO: 64 - DNA, 2302 bp, Mus musculus
  CDS (339)..(1475)
tgaaactattaaattccttgctcagatttcaggaagtaaagtgtgctgttcatctcaatctctcctgtctaacccctcccctcccgatttccgggggatcaatgatagtagagagctttggggcctctggaaatcctgtggggccctgtcacttttggtccttgtatggagtcctgctaggtgtccactggagtgtgttacatctcgggacctttagaggaattcggagtgcggggctgtggctgctgtctccccattcagaagccacttgctagtagctactgaaaggctcttcattgtctcttctgctccaggaacaccggtctaggaagcagaagatgccatactccaacctgcatccagccatcccacggcccagaggtcaccgctccaaatatgtagccctcatctttctggtggccagcctgatgatcctttgggtggcaaaggatccaccaaatcacactctgaagtacctagcacttcacctagcctcgcacgaacttggactactgttgaaaaacctctgctgtctggctgaagagctgtgccatgtccagtccaggtaccagggcagctactggaaggctgtgcgcgcctgcctgggatgccccatccactgtatggctatgattctactatcgtcttatttctatttcctccaaaacactgctgacatatacctcagttggatgtttggccttctggtcctctataagtccctaagcatgctcctgggccttcagagcttgactccagcggaagtctctgcagtctgtgaagaaaagaagttaaatgttgcccacgggctggcctggtcatactacattgggtacttgcggttgatcttaccagggctccaggcccggatccgaatgttcaatcagctacataacaacatgctcagtggtgcagggagccgaagactgtacatcctctttccattggactgtggggtgcctgacaacctgagtgtagttgaccccaacattcgattccgagatatgctgccccagcaaaacatcgaccgtgctggcatcaagaatcgggtttattccaacagcgtctacgagattctggagaacggacagccagcaggcgtctgtatcctggagtacgccacccccttgcagaccctgtttgccatgtcacaggatgccaaagctggcttcagtcgggaggatcggcttgagcaggctaaactcttctgccggacacttgaggaaatcctggaagatgtccccgagtctcgaaataactgccgcctcattgtctaccaagaacccacagacggaaacagtttctcactgtctcaggaggtgctccggcacattcgtcaggaagaaaaggaggaggttaccatgaatgcccccatgacctcagtggcacctcctccctccgtactgtcccaagagccaagactcctcatcagtggtatggatcagcctctcccactccgcactgacctcatctgaggcatgggacagccttgtctgggctctagtgatcctttagcctcctgactgagccttccttcaatggttgggggcctcagagacttcacatctccagatgagtcccacattcctgggcaagccatttatttcacctctctgagcctcaaccaaccctactatgaaaggaggtcataatgcgttccctgcccagccaaaggattttatatatgtagaagttggtgtcaatgcctggtaaacttgagagaaaggccaagtacttcccgtggatgctgcagacattccctgctctctgttgacctgtgtggatggtaccagcagacttccaaccctccagcttctggtcacgtgtgttcaatgggagcttaagtagatggcgagagggagaaggaacatttgttctgttagctgtatacaatcacagtgggctggcctgtcaactgccttcttaataaacatatctattctcagatttctagaatggcctcttccccttgtctctagcactggtatttgtgtgacactggagtactttctgtctggtctctttatatcatgtcccttgcacatggtgttggcatcaggacgtcccaaactcatgacatcacataggcgacagcatgacctgcaacctgcagaccggttgccaagacaacaggcaccatattcccaccttccacttggctcacctcccacctttacctgtgttacgtcatcttccatatcttccatacgtcttccatcttccatacgtctctctcccctgcttctctttctgctgctaccttgtctctcccttccaataaaacctcttccatgcggaactg

SEQ ID NO: 65 - protein, 378 aa, Mus musculus
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 66 - DNA, 2196 bp, Homo sapiens
  CDS (81)..(704)
actcggccttctgggcgcgcgcgacgtcagtttgagttctgtgttctccccgcccgtgtcccgcccgacccgcgcccgcgatgctggcgctgcgctgcggctcccgctggctcggcctgctctccgtcccgcgctccgtgccgctgcgcctccccgcggcccgcgcctgcagcaagggctccggcgacccgtcctcttcctcctcctccgggaacccgctcgtgtacctggacgtggacgccaacgggaagccgctcggccgcgtggtgctggagctgaaggcagatgtcgtcccaaagacagctgagaacttcagagccctgtgcactggtgagaagggcttcggctacaaaggctccaccttccacagggtgatcccttccttcatgtgccaggcgggcgacttcaccaaccacaatggcacaggcgggaagtccatctacggaagccgctttcctgacgagaactttacactgaagcacgtggggccaggtgtcctgtccatggctaatgctggtcctaacaccaacggctcccagttcttcatctgcaccataaagacagactggttggatggcaagcatgttgtgttcggtcacgtcaaagagggcatggacgtcgtgaagaaaatagaatctttcggctctaagagtgggaggacatccaagaagattgtcatcacagactgtggccagttgagctaatctgtggccagggtgctggcatggtggcagctgcaaatgtccatgcacccaggtggccgcgttgggctgtcagccaaggtgcctgaaacgatacgtgtgcccactccactgtcacagtgtgcctgaggaaggctgctagggatgttagacctcggccaggacccaccacattgcttcctaatacccacccttcctcacgacctcatttctgggcatctttgtggacatgatgtcacccaccccttgtcaagcattgcctgtgattgcccagcccagattcatctgtgccttggacatggtgatggtgatgggttgccatccaagtgaaagtcttttccttgaccaagggggacagtcagttttgcaaaaggactctaatacctgtttaatattgtcttcctaattgggataatttaattaacaagattgactagaagtgaaactgcaacactaacttccccgtgctgtggtgtgacctgagttggtgacacaggccacagaccccagagcttggcttttgaaacacaactcagggcttttgtgaaggttcccccgctgagatctttcctcctggttactgtgaagcctgttggtttgctgctgtcgtttttgaggagggcccatgggggtaggagcagttgaacctgggaacaaacctcacttgagctgtgcctagacaatgtgaattcctgtgttgctaacagaagtggcctgtaagctcctgtgctccggagggaagcatttcctggtaggctttgatttttctgtgtgttaaagaaattcaatctactcatgatgtgttatgcataaaacatttctggaacatggatttgtgttcaccttaaatgtgaaaataaatcctattttctatggaagactggtacctggtttctggaagaggggtctgtgacttggagctgatctttactgagctcgccgtggcagatgccatgctcaggacgttcatgtggatggtttcatgtcatcgtgctggcaacttgtcctccctgccttagagatgaggctcagacaaacgaccttagcacccatagcctatgccatgagcactggctccaccctgaatcccagctcctccccttagtgaccccaagtctgtttccctcagctgcataaggaggcgatatagtttgaatatttgtccccagccaaatctcatgttgaactgtaatccccagtgctggaggtggggcctgctacgaggtgtttggatcatggggacgggtatttcatggcttggtgctgttttcttgatggtgaattattgcaagatacggtcatttaaaattgtgtggcacctccccctgcccccttcttgctcctgctttcaccatgtgacatgcctgatcccccttcaccttttgccatggtcataagcttcctgaggcctccctggaagctgagcagatgccagcaccatgcttcctgtacatcctgcagaaccataagccaattaaacctttttaataataaa

SEQ ID NO: 67 - protein, 207 aa, Homo sapiens
MLALRCGSRWLGLLSVPRSVPLRLPAARACSKGSGDPSSSSSSGNPLVYLDVDANGKPLGRVVLELKADVVPKTAENFRALCTGEKGFGYKGSTFHRVIPSFMCQAGDFTNHNGTGGKSIYGSRFPDENFTLKHVGPGVLSMANAGPNTNGSQFFICTIKTDWLDGKHVVFGHVKEGMDVVKKIESFGSKSGRTSKKIVITDCGQLS

SEQ ID NO: 68 - RNA, 58 nt, Artificial Sequence
  Artificially synthesized inhibitory RNA
ccggcuguggccaguugagcuaauccucgaggauuagcucaacuggccacaguuuuug

SEQ ID NO: 69 - DNA, 1560 bp, Mus musculus
  CDS (63)..(683)
tacgaccttgcctgtgtctgctctgagttcttccgcgcgccctcgcccgacccgcgacagcgatgctagcgctgcgttgcggcccccgcctgctcggtctgctctccggcccgcgctccgcgccgctgctcctctccgcgacccgtacctgcagcgacggcggagcccgcggcgcgaactcttcctccgggaacccgctcgtgtacttggacgtgggcgccgatggacagccgctcggccgcgtggtgctggagttaaaggcagatgtcgtgccaaagactgcagagaacttcagagccctatgcactggtgagaagggctttggctacaaaggctccaccttccacagggtgatcccagccttcatgtgccaggctggcgacttcaccaaccacaatggcacaggagggaggtccatctacggaagccgctttcccgacgagaacttcacactgaagcatgtggggccaggtgtcctgtccatggcgaacgcaggccccaacaccaatggctctcagttctttatctgcacgataaagacagactggctagatggcaagcatgtcgtgttcggccatgtcaaagagggcatggatgttgtgaagaaaatagaatctttcggctcaaaaagtgggaagacgtctaagaagattgtcatcacagactgtggccagttgagctaactcacagccaaggtgctaggacagcagcaggcatccatgtcttgattcacccaggttctccaaagaacagtttgcacccacttctattgagtctgaggaaggccactcaggcatggtcctcccagaccaggctgctctccactgtccatcctgcctcagatcccgtttctgggcatcagtatggccatcgggccatgtaccctcagccgatgtccccgtgattgccatgtgcgtgtgccttggacattggcaatgctgaccagttagtcagggaaggctcctgactcttttccttgatcagtggggatagcagttgctatttaatgttgtcttcctccttgcaataatttaacatagaattcaagatctcatcgaaacaaagctgtgacactcatggcactgtccgtgtgtgacctgtgttgctgtgacactcacggcactgtccatgtgtgacctgagttggcacaagccacagacacccccaacccccccacctcccagcctggcttctgagacatcactcagggctcttgtgaaagtcccagcgtcaatgccttccctcctggtcactgtgaatcctgctggctgctgctgtgatttttgaaagcctgtgaaatagaagcagcaggacctgggaacagacctgtgagcccggtctttctgctcaatgtgaattcccgtggtgaaaacttacagacactgcctgacctcatgctgttctcaagagggaagtgtcttctggcaggctttgctttttctgtatgttgaataaatccaatctatcaataatatatatgtaaaagatttctgggacagtcctgtgtgttcatctcaaatgtgataaataaatcctattttctataaaaaaaaaaaaaaa

SEQ ID NO: 70 - protein, 206 aa, Mus musculus
MLALRCGPRLLGLLSGPRSAPLLLSATRTCSDGGARGANSSSGNPLVYLDVGADGQPLGRVVLELKADVVPKTAENFRALCTGEKGFGYKGSTFHRVIPAFMCQAGDFTNHNGTGGRSIYGSRFPDENFTLKHVGPGVLSMANAGPNTNGSQFFICTIKTDWLDGKHVVFGHVKEGMDVVKKIESFGSKSGKTSKKIVITDCGQLS

SEQ ID NO: 71 - DNA, 1723 bp, Homo sapiens
  CDS (111)..(1565)
gcagtttcacttttagctctgggcacctccagctcctgctcgccggacggctcccagggagagcagacgcgccagacgcgccaccctcggggcgccgacggtcacggagcatggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctgcagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagatggggtccctgcggagggggcgttcactgaagacttccagggcctacgggcagaggtggagaccatctccaaggaactggagcttttggacagagagctgtgccagctgctgctggagggcctggagggggtgctgcgggaccagctggccctgcgagccttggaggaggcgctggagcagggccagagccttgggccggtggagcccctggacggtccagcaggtgctgtcctggagtgcctggtgttgtcctccggaatgctggtgccggaactcgctatccctgttgtctacctgctgggggcactgaccatgctgagtgaaacgcagcacaagctgctggcggaggcgctggagtcgcagaccctgttggggccgctcgagctggtgggcagcctcttggagcagagtgccccgtggcaggagcgcagcaccatgtccctgccccccgggctcctggggaacagctggggcgaaggagcaccggcctgggtcttgctggacgagtgtggcctagagctgggggaggacactccccacgtgtgctgggagccgcaggcccagggccgcatgtgtgcactctacgcctccctggcactgctatcaggactgagccaggagccccactagcctgtgcccgggcatggcctggcagctctccagcagggcagagtgtttgcccaccagctgctagccctaggaaggccaggagcccagtagccatgtggccagtctaccatggggcccaggagttggggaaacacaataaaggtggcatacgaaggaaa

SEQ ID NO: 72 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 73 - RNA, 59 nt, Artificial Sequence
  Artificially synthesized inhibitory RNA
ccggcaaccugucuaucaaggacaucucgagauguccuugauagacagguuguuuuuug

SEQ ID NO: 74 - DNA, 1776 bp, Mus musculus
  CDS (148)..(1611)
gtgtcctgccgcctgagttccgctcttggtcgtggctcccgttgctcccgggttgagcagacaatagacccctccccggcatcccagcaggtcctcgcttcgcttggtggacccagatacctcggcaggggtgaaaaatcgaggaccatgccatcggcctttgagaaagtggtcaagaatgtgatcaaggaggtaagcggcagcagaggcgatctcattccggtggacagcctgcggaactccaccagcttcaggccctactgccttctgaacaggaaattttcaagctcaaggttctggaaaccccgttattcatgtgtcaacctgtcaatcaaggacatcctggagcccagtgctccagaaccagaaccggagtgttttggctccttcaaagtctctgatgtcgtcgatgggaacattcagggcagagtgatgttgtcaggcatgggagaagggaaaatttctggtggggctgcagtgtctgacagttccagtgcctccatgaatgtgtgtatactgcgtgtgactcagaagacctgggagaccatgcagcatgaaaggcaccttcagcagcctgagaacaaaatcctgcaacagcttcggagtcgtggggatgacctgtttgtggtgaccgaggtgctgcagacaaaggaggaagtgcagatcactgaggtccacagccaagagggctcaggccagtttacgctgcctggagctttatgcttgaagggtgaaggcaagggccaccaaagccggaagaagatggtgaccattcctgcaggcagcatcctggcattccgagtggcccaactgcttattggctctaaatgggatatccttctcgtctcagatgagaaacagaggacctttgagccctcctcaggtgacagaaaagcagtgggccagaggcaccatggcctcaatgtgcttgctgcgctttgttccatcggaaagcagctcagtctcctgtcagatgggattgatgaggaggaattaattgaggcggcagacttccagggcctgtatgctgaggtgaaggcttgctcctcagaactggagagcttggaaatggagttgagacaacagatactggtgaacatcggaaagattttacaggaccagcccagcatggaagccttagaggcctcactagggcagggcctgtgcagtggcggccaggtggagcctctggacggcccagctggctgcatccttgagtgtctggtgcttgactctggagaactggtgccggaactcgcagcccctatcttctacctgctgggagcactggctgtgctgagtgaaacccagcagcagctgctagctaaggctctggagacaacggtgctgtcaaagcagctggagttggtgaagcacgtcttggaacagagcaccccgtggcaggagcagagttctgtgtccctgcccaccgtgctccttggggactgctgggatgaaaagaatcccacctgggtcttgctagaagaatgtggcctaaggctgcaggtagaatccccccaggtgcactgggaaccaacgtctctgatccccacaagtgcgctctatgcctccctgttcctattgtcaagtctaggccagaaaccttgttagcctgtgggcctcccttcccacaacatctccatgtcctaccctccagccaaggtagaatcttgccaagcctagcctttgggaagccaagaaccatactcagtcacagggttataatgcactgagatccagaagttgggaaaactcaataaatgtacaaaggaaagc

SEQ ID NO: 75 - protein, 487 aa, Mus musculus
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDGIDEEELIEAADFQGLYAEVKACSSELESLEMELRQQILVNIGKILQDQPSMEALEASLGQGLCSGGQVEPLDGPAGCILECLVLDSGELVPELAAPIFYLLGALAVLSETQQQLLAKALETTVLSKQLELVKHVLEQSTPWQEQSSVSLPTVLLGDCWDEKNPTWVLLEECGLRLQVESPQVHWEPTSLIPTSALYASLFLLSSLGQKPC

SEQ ID NO: 76 - DNA, 839 bp, Homo sapiens
  CDS (74)..(637)
attctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtcaa

SEQ ID NO: 77 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 78 - RNA, 57 nt, Artificial Sequence
  Artificially synthesized inhibitory RNA
ccggcagaguggaaauccuaaggaacucgaguuccuuaggauuuccacucuguuuuu

SEQ ID NO: 79 - DNA, 770 bp, Mus musculus
  CDS (21)..(569)
acaccagcctggcttccatcatgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactgaagacctgtcagttgatgcctcagaatgagtggtggttgcaggcaacctttaagcatcagaggcggactctgggactggtagtgaatctactgcatttgaaaggtcaaaggaaaacagagtttttattaatttataatttaaattattttctactttttatttaaactttttaacctcagaaaataaaatatttataataca

SEQ ID NO: 80 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 81 - DNA, 3868 bp, Homo sapiens
  CDS (87)..(1763)
ggtgtgacttaggacggggcgatggcggctgagaggagctgcgcgtgcgcgaacatgtaactggtgggatctgcggcggctcccagatgatggtcgtcctcctgggcgcgacgaccctagtgctcgtcgccgtggcgccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttagttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaaatttggcttatagttggaatttgtattgcattatttgctctcccgtttgtcatttatgctgcgaaagtcttcttgagatgcatcaattatgtcttctttccatcacttaaaccttcttccagtatagatgagtatttctctgaacagccattgaagaatcttctgctttcaacttctgaggaacaaatcgaaaaatgtttcataattgaaaatataagcacaattgctacagtagaagaaactaatcaaactgatgaagatcataaaaaatacagttcccaaactagccaagattcaggaaattattctaatgaagatgaaagcgaaagtaaaacaagtgaagaactacagctggtcttggactcctgacctcatgctccacccgcttcggcctcccaaagttctgggattacaggtgtgagccaccgtgcacggccggcctgacctttggaaaagccttgtcactttggacgtttgcgtctttgaagaggcgatgggagcatatcatgactgcctgccaccattgcttttcagactaccacaactcaatcatgctgtccaggacttctggccctgtgttcaccactgggaaaacgtacttcagactggatagcctaaaaaggagcaatgcccttgtaggatgtggagaagggaaaatacggacattaacattaaaagacaccagtgaaattgttaggtctctaggaagttggagcacaaggcttcacgctttaagaccatctgtggttttcagtgaacaagcgctgagcaccagcagcagaaaacaacaacaaaaaaacacctcgtttttaccttgtcttctagacatgaaaaggcagttgcattccactctgcattatgttctacatgttgctttatcagtatatgcttagctgtaagtgacaagtattttttctgaacagaagtttacttagaaataccatgcacttgggggtaccaattaaccgcctgaaaattagcatattgatagttcttagagagaccagatataatctaagaatttatatgaaagatttgtatcattagagccagaaataattttatattaatatataatacagattaacattatatataatatgtacctgtgtcacttctgacatgagcctgtaaacatatattcatatatgtacctgcacatgtacccacctgatgtaggtcttattcctttagtatggacttaaagtacttattcatataccttgtaactaaaaattagaacagctccctagaattgtgaacttttaagagtctgactagaaatttgcaacttataaaaaagttacttttaaaaatataagttagggctaggcacagtggctcatgcctataatctcagcacttttgggaggccaagacaggaggatcacttcaggccaggagttcaagatcaaccaacctgggtaacatggccagaccccatctctatttatatatatatatataaaacttagagtttttatcttcccctaaaagaggccgtgatatttgcagcagcctcaaattgctcttaaggggtttaggtgtgcagaagctttcctttccctacccagtaaccatgtgactactaacgtggtatattgatttattttgtttgctgtctgtctcccctgccccactgctggaacagaggctccaagaaaacagggaccttattattcattactgcatccccagtaatgaaagtacttagaaaataattattgaatgaatgaaatctaaactgtgaacctgagggtgtttgtggcagtgtttgttttactgaattgtagaaggacataaccgtgttttcagtgtttctatggaacaaacttgtacattttatttcacttgtgttttgtcttaaaccctactgctggaaacaattttatgtaataagcaatgggcccaaaagtctaggagtttttttgtacttagtgaatttgtatgcaacagagatgctgcagctgatgcctttaaaaggtattcatcatggaagagctgaggcctgtgcttggtgttccagagcccagggttgagcatcctgaaggagccactgcagccgtcactgtccccagagcctgtggagatagagcctgtttgctgctttttcttcccgctcttaagacatggctggagctcagtcttcattgaatgaagtttgctgtggtattgcatagccttgctttcttgaactaaactgtttgcccttcacaagtagttcttctttcaggattagttcgttccaaggaggctcttcagtctcacagataagtagatctctcctgctgtctggacacatttcactcggaaattgaatacaatttgtattcaggctgggaacctgaacacacacttgtgtttttaagcttcccttttttacagtggacaaggacacaaataataaataaatcatccctaatgcccaagaaatgccctggtacttagtaataacaaaataccagtaacttcca

SEQ ID NO: 82 - protein, 558 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQLVLDS

SEQ ID NO: 83 - RNA, 58 nt, Artificial Sequence
  Artificially synthesized inhibitory RNA
ccgggccaagauucaggaaauuauucucgagaauaauuuccugaaucuuggcuuuuug

SEQ ID NO: 84 - DNA, 7025 bp, Mus musculus
  CDS (130)..(1902)
ggaaggggcggggcgctggagggggcggggcgtcgcggagggcctagctgcccagaggtagtctccagctccgcggtgctgctgaggagaaggaggagaatgtgagccgccgcccggcctcccaagacgatgctcgctgtcgtgggcgcggcggccctggtgctggtggccggggcgccttgggtgctaccctcagctgcaggtggagaaaatctgaaacctcctgagaatatagacgtctacattatagatgacaactacaccctaaagtggagcagccacggagagtcaatgggcagtgtgaccttttcagcagaatatcgaacaaaagacgaggcgaagtggttaaaagtgcctgaatgtcaacatactacaacgaccaagtgtgaattctctttactggacacaaatgtgtatatcaaaacacagtttcgtgtcagagcagaggaagggaacagcacatcttcgtggaatgaggttgatccgtttattccattctacacagctcacatgagccccccagaagtacgtttagaagctgaagataaagccatactagtccacatctctcctcccggacaagacgggaacatgtgggcactggagaaaccttccttcagttacaccatacgaatctggcagaagtcttccagtgacaaaaaaactattaactctacgtattatgtagaaaagataccagaactcttgccagagactacttactgtttagaagttaaagcaatacatccgtcacttaagaaacacagcaattacagcactgtgcagtgtataagcaccacagtggcaaataaaatgcctgtgccaggaaatctccaagtggatgcccaaggcaagagctatgtcctgaaatgggactacattgcgtctgcagacgtgctcttcagggcacagtggcttcctggctattcaaaaagcagttctggaagccgttcagataaatggaaaccaataccaacctgtgcaaatgtccagactacgcactgtgtcttttctcaagatactgtctacacaggaacgttctttctccatgtacaagcctcagagggaaatcacacatccttttggtctgaagagaagtttattgattctcaaaaacacattctccctcctcctccggtcattactgtcaccgccatgagtgacaccttgcttgtttatgtcaactgtcaggacagcacatgtgatggactcaattacgaaatcatcttttgggaaaacacttccaatactaagataagcatggagaaggatggcccagagttcaccctcaagaacctgcagccgctgactgtgtactgtgtccaggccagagtgctcttcagggccctgctgaataagaccagcaacttcagtgaaaagctgtgtgagaaaacacgtccaggaagtttttccacgatctggattataactggattaggtgttgtgttcttctctgtcatggtcctttatgctttgaggagcgtctggaaatacctgtgtcatgtgtgcttcccaccactcaagcctccccgcagtattgatgagtttttctctgagccgccttcaaaaaaccttgtacttctgacggctgaggagcacacggaaagatgcttcatcattgagaatacagacacggtcgctgtagaagtaaagcacgcgcctgaggaggacctcaggaagtacagctcacagaccagccaggactcgggcaactattccaacgaagaggaggagagtgtgggcaccgagagcggccaagctgtgctctccaaagctccctgcggggggccatgcagcgtgcctagccctcctgggaccttggaagacgggacctgcttcctgggaaatgaaaaatatcttcagagcccagccctgaggacagagccagctcttctctgctgacaggtgctcggcccaggctgggggccgtgtggaggagacagacttatctgaagtggctgccagcttctgctgccagctggaagccctgggtgctgtttgtgccacagatgaggctcactgatgtcaggcatctggtggttctgtctcggtgtttgtcctctaggtgacatttagaacattccgatttttaatcctgccgtagcccctcagtgtgtcacaggtaccattgtctacagccgcgcttcctaacccatacctcaccacagccgtggcttctgaccagaagtcactgaaaccatggagaaaggggacactaactggaggagctggcgttgtttaagatctcagtgtggcctgggtgtgcccgttgtactctagtacttgcagaactagaaaatgccagggacaaaaagcaggcatgaaccattcagtaagagagaagctagtgaacctgatggtgaaagaagaccgtgtccagtcagtgtttgtgtgaaggccctgtgctgctctctgtagccttcccgtggtacttataaaaccaggccgaaccctgtcctctgtccggcactattcaggtcccttcccagctccaggtacttaaaaaacaaagtgttgggtcctggggtgagggaacagcttcgacggcacagtgctgcctgcaggcgcctataagtgaccttagatgaatgctccatgagcatcttcggtaggcaggacatccagcagtgctggtggctccagggcagactcagttgcaggccaaaaaggaagagagaggagtcatagccctgccccaatcgagcttgttgctcgctgcactgcgcctttgtaatcaatcttggtttttttgttttgttttgttttttttaacttttaggtgataacatattctcagatatagcacacaagagacactacacaaattacgtctgaaagttttgatagaaaagagcttttgactggggtagacagcttgctcctcactactaccttgtatctcagtctcctcaacagagagagaatgcagattgattttcatcctgagttttattttcttgcccggtttctgggttcgggtgtgctttcaaggctctgaacagcttgtctgcccacaggggagcccggagacccagccttttggctaaatgttgcaggttcctgtacttgaaggactccagctcctctgtagagggcatggtcatcgcggtctgcaatcacgctgatacccagaaaaatggccacacaactcaaatctggtgccacgctttaataattctacccagcacggcccagccctgttcacggcggaaaagctccttctgccaggacagcctgtgaagaaacaagacctccgaggacgtggccaaggaaggcccagatgctagcgggaagttgttagttggcactctttcatctctcaacaactgtgacacaggtcttcagggtgaagcccaaagcagctatcggttgcagcagcgggtaaggagggggcttcacctgagcctggccttctgggcgagcagttctacctgcacacttcaagacagcctccatgacccttttcacggcacaccgcctgcttttggtgtgaccggggtttcctgtgcaggcacatgttcttaaggaatgccatgcacacaggcctgaagagctggctcagcgggtagagcacaggttgctcttgtctgggatctgagctctgtccccagcacccacagagtgcttctggttgcctcagactccaggggtgcacaaggccatgtaagcaaagacatgtaagcaaagacagttgtacacagagaagtcaaacctttttgaaaaaaagaaacaccacgggctggagagatggctcagtggttaagaacactgaccactcttccagaggtcctgagttcaattcccagcaaccacatggtggctcacaaccatctgtaataggatctgatgcccccttctggtgtgtctgaagatagctacagtgtattcatatataaaataataaatacatccttttttttctttttttactattggatattttctttatttacatttcaaatgttatcccctttcctgatttacccccctcccagaagccccctatcccatcccccctcccctgcttctatgagggtgttcacccacccacccactcccacttccctgcccttgattcccctacactgggttatttatcaagccatcataggaccaaagacctctccttccattgatccatgacaaagccatcctctgcaacatatgcagcttgagctatgtgtacttcttggttgatggcatagtccctgggagttctggggcagtggggggttggggggagtctggttagttgatattattcttcctattggggttgcaaaccccttcaatgccttcagtcccttctctaactcctctattagggaccccgagctcagtccagtggttggctgctaacatccacctctgtatttgtaaggctctggcagggcctctcaagagacagccatatcaggctcctttcagcatgtacttcttggcacccacaacagtgtctggtttggtaacgctgtacggcatgaattccgaggtgggacaatctatgggtggcctttcattcagtctctgctctacactttatctccatatttgctccagtgagtattttgttctccttctaagaaggacagaagcacccacactttgctcgtccttcttattgagcttcatgtggtctgtgaattgtatcctggttatttggagctttggggctagtatccacttatcagtgagtacatattgtgtgtgttcttttgtgattgggttacctcactcaggatgatatcctccagatccatccatttgcctaagaatttcatgaattcatcgtttttaatagctgtgcagtactctgttgtggaaatgcaccacgttttctgtatccattcctctgttgaggggcatctgggttctttccagcttctggctattagaaataaggctgctatgaacatagtggagcatgtgtccttcttaccagttggagcatcttctgggtatatgcccaggagtagcatagctgggtcctcaggtaatactatgtccaattttctgaggaccggccaaactgatttccagagtggttgtacgagcttgtattcccaccagcaatggaggagtgttcctctttctccacatcctcaccaacatctgctgtcaacagagtttttaattttagccattctgactggtgtgaaatggaatctcagggtggttttgatttgtaattcaccaatgactaaggatgttgaacatttctttaggtgcttcttggccatttgatattcctcagttgaaaattctttgtttagctctctaccccattttttaatagggttatttggttctctggagtctaacttcttgagttctttgtatatattggatattagccctctatcggatgtaggattggtaaagatcttttcccaatctgttggtattgttttgtccttttgtcagtgtcctttgccttacagaagctttgcaattttatgaggtcccacttgtcgattctttttttttttttttttttttttttttcgagacagggtttctctgtatagccctggctgtcctggagctcactttgtagaccaggctggcctcgaactcagaaatccgcctgcctctgcctcccgagtgctgggattaaaggcgtgcaccaccacacccggctccatttgtcgattcttgatcttacagcattagccattggtgttctattaaggaattttttcccctgtacccgtatgtttgaggctctttccaaattgctcctctataaagagagagagggggagagggagaaggagagagcaaggaaggaagaaaagagaagagacaagacaagaaggaaaaggagagaaaagaaaagagaaaaaagaaatgaaaagaagagaagagaaaagagaaaaaagaaaagaaagaccatgcacttgagggcactttgtttatgttgatagttcttggagaagctgagcgccgtctaagattcatgtaaaagcctcatgagaaatcagtgctttcacagacacatgtaaatagcaacaggcctgtgagcacagctacacaggggagctggctgcactccactagaattgccacgacgtacctatcacagtgacgtctacagtgagaaatgctgttctagactcattacatatgtaatcatttaaatgtaatcatttagagcctgggcagaaatctataacctatgaaaatgttcatttaaaatcaggtcaggcacaagtgtgcctggcttgttctcatcccagctgcctaggaatgtgggccaacctcagccacatagcaagagcctgtcgtacacatatgtacaggtgtagaggtatgtgtgcatgtatataggtactatataaaatacagagcccagagcctgtgatgcttcagatggcctcggacttctctggagacctggggaacgtgttaacaaagccttccctaaccccatggaagccgtgtgtccctgtgtgcattggtcctttggtgtttgcctccctctgtattgcagaagctctttgagcagggtctcagttcctccctgtggccccccagcccctgtgacagtgcagcgctggcacagacagtgcccacagctaccgaatgaatgagatctgaaattgaagcagatgactgttctggctgtttgtgtaatgaaataaccacgtttgtgtgtttctatgcatcacctttgtgtctgcatccttttatatttttgtctggagccccactgttgtatatcgacaaacctaacacacaccggaaccaggactctggatctcaggtgctgctcctttaaactggaatccgtgaggcagaggtgccgtctacaacccgttcctgacagcaggacttgcattctggcattctggatccacactgctgtcccttccaagtgttactgtctctcattcctctgtctctgagatcccttctgctctacctcaagagactaagagggtgaacacagtttgtacttttcatcttccttacagagaaggaaataataaattcatatttgttcccaagaaaaaaaaaaaaaa

SEQ ID NO: 85 - protein, 590 aa, Mus musculus
MLAVVGAAALVLVAGAPWVLPSAAGGENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHILPPPPVITVTAMSDTLLVYVNCQDSTCDGLNYEIIFWENTSNTKISMEKDGPEFTLKNLQPLTVYCVQARVLFRALLNKTSNFSEKLCEKTRPGSFSTIWIITGLGVVFFSVMVLYALRSVWKYLCHVCFPPLKPPRSIDEFFSEPPSKNLVLLTAEEHTERCFIIENTDTVAVEVKHAPEEDLRKYSSQTSQDSGNYSNEEEESVGTESGQAVLSKAPCGGPCSVPSPPGTLEDGTCFLGNEKYLQSPALRTEPALLC

SEQ ID NO: 86 - RNA, 290 nt, Artificial Sequence
  Artificially synthesized Alu RNA
ggccgggcgcgguggcucacgccuguaaucccagcacuuugggaggccgaggcgggcggaucacgaggucaggagaucgagaccauccuggccaacacggugaaaccccgucucuacuaaaaauacaaaaauuagccgggcgugguggcgggcgccuguagucccagcuacucgggaggcugaggcaggagaauggcgugaacccgggaggcggagcuugcagugagccgagaucgcgccacugcacuccagccugggcgacagagcgagacuccgucucaaaaaaaaaa

SEQ ID NO: 87 - RNA, 145 nt, Artificial Sequence
  Artificially synthesized B1 RNA
  misc_feature (89)..(89): n is a, c, g, or u
gccgggcaugguggcgcacgccuuuaaucccagcacuugggaggcagaggcaggcggauuucugaguucgaggccagccuggucuacanagugaguuccaggacagccagggcuacacagagaaacccugucucgaaaaaaaaaa

SEQ ID NO: 88 - RNA, 209 nt, Artificial Sequence
  Artificially synthesized B2 RNA
gggcuggagagauggcucagugguuaagagcaccugacugcucuuccagagguccugaguucaauucccagcaaccacaugguggcucacaaccaucuguaaugagaucugaugcccucuucuggugugucugaagacagcuacaguguacuuacauauaauaaauaaauaaauaaauaaaucuuaaaaaaaaaaaaaagaaagaaaaa

SEQ ID NO: 89 - RNA, 19 nt, Artificial Sequence
  Artificially synthesized siRNA
ggaaauuacaguuagaggu

SEQ ID NO: 90 - RNA, 19 nt, Artificial Sequence
  Artificially synthesized siRNA
gacaaguugauaggauaua

SEQ ID NO: 91 - RNA, 19 nt, Artificial Sequence
  Artificially synthesized siRNA
ccaaucugauguaucagga

SEQ ID NO: 92 - RNA, 19 nt, Artificial Sequence
  Artificially synthesized siRNA
gcaguuguccuaaacagau

SEQ ID NO: 93 - RNA, 19 nt, Artificial Sequence
  Artificially synthesized siRNA
caacugggcuccucuguaa

SEQ ID NO: 94 - RNA, 19 nt, Artificial Sequence
  Artificially synthesized siRNA
guaaagagcuauauggaua

SEQ ID NO: 95 - RNA, 19 nt, Artificial Sequence
  Artificially synthesized siRNA
ggcuagauguggaagaugu

SEQ ID NO: 96 - protein, 14 aa, Homo sapiens
RYLEDLEDVDLKKF

SEQ ID NO: 97 - protein, 16 aa, Homo sapiens
KQQMESGKSLAQTSKT

SEQ ID NO: 98 - protein, 14 aa, Homo sapiens
RMNLFQKEVDCEKF

SEQ ID NO: 99 - protein, 24 aa, Homo sapiens
RQFGALTAEVGDMTEDSAQALIRE

SEQ ID NO: 100 - protein, 13 aa, Homo sapiens
RYTCGSSVEATRA

SEQ ID NO: 101 - protein, 15 aa, Homo sapiens
RLPGGLTDSLGNLKN

SEQ ID NO: 102 - protein, 13 aa, Homo sapiens
KILAQNLHNLVKL

SEQ ID NO: 103 - protein, 10 aa, Homo sapiens
RILGAFFGKN

SEQ ID NO: 104 - protein, 12 aa, Homo sapiens
KEFLPDPALVRK